(19) United States
(12) Patent Application Publication
(10) Pub. No.: US 2014/0370521 A1
(43) Pub. Date: Dec. 18, 2014

(54) IDENTIFICATION OF TWO NOVEL BIOMARKERS FOR NIEMANN-PICK DISEASE TYPE C

(71) Applicant: The USA , as Represented by the Secretary, Department of Health & Human Services, Rockville, MD (US)

(72) Inventors: Forbes D. Porter, III, Gaithersburg, MD (US); Celine V. M. Cluzeau, Bethesda, MD (US); Dawn E. Watkins-Chow, Kensington, MD (US); Christopher A. Wassif, Edgewood, MD (US); William J. Pavan, Derwood, MD (US)

(21) Appl. No.: 14/365,398

(22) PCT Filed: Dec. 14, 2012
(86) PCT No.: PCT/US12/69955

§ 371 Date: Jun. 13, 2014

Related U.S. Application Data
(60) Provisional application No. 61/576,062, filed on Dec. 15, 2011.

(51) Int. Cl. G01N 33/68; G01N 33/573

(52) U.S. Cl. CPC G01N 33/6893; G01N 33/573; G01N 2333/96475; G01N 2333/47; G01N 2800/044
USPC 435/7.4; 436/501; 530/389.8

(57) ABSTRACT

This invention provides novel biomarkers for Niemann-Pick disease, type C (NPC). In an exemplary embodiment, the invention provides methods for identifying a subject as having NPC. In embodiments, the methods involve detecting the level of biomarker selected from the group comprising i) galectin-3 (LGALS3); ii) cathepsin D (CTSD); iii) LGALS3 and CTSD; and iv) LGALS3 and/or CTSD in combination at least one additional NPC associated biomarker in a sample obtained from the subject. In embodiments, the methods involve comparing the level of biomarker to a reference.

### CROSS-REFERENCE TO RELATED APPLICATIONS

This application claims the benefit of U.S. Provisional Application No. 61/576,062, filed Dec. 15, 2011, the contents of which are hereby incorporated by reference in its entirety.

### STATEMENT OF RIGHTS TO INVENTIONS MADE UNDER FEDERALLY SPONSORED RESEARCH

This work was supported by the Eunice Kennedy Shriver National Institute of Child Health and Human Development intramural research program, the Therapeutics for Rare and Neglected Diseases program of the National Human Genome Research Institute intramural research program, and a Bench to Bedside award from the Office of Rare Diseases. The Government has certain rights in this invention.

### BACKGROUND OF THE INVENTION

Niemann-Pick disease, type C (NPC, OMIM #257220) is a lethal, autosomal recessive, neurovisceral disorder characterized by intracellular accumulation of unesterified cholesterol and glycosphingolipids in late endosomal/early lysosomal compartments. NPC has a wide clinical spectrum with a variable age of onset. The earliest clinical findings are often related to liver disease, usually cholestasis with prolonged neonatal jaundice and hepatosplenomegaly. Although cholestasis resolves in many of the patients, some of them develop chronic liver disease and can die of liver failure. Neurological features are progressive, and include ambulatory impairment, ataxia, dementia, dysarthria, dysphagia, seizures, and supranuclear vertical gaze palsy. The incidence has been estimated at 1/120,000-150,000 in Western Europe. Ninety-five percent of NPC cases are due to mutations in the NPC1 gene (18q11), which encodes a large transmembrane protein localized in the late endosomal/early lysosomal compartment which functions in intracellular cholesterol transport and homeostasis. The remaining NPC cases are due to mutations in the NPC2 gene (14q24.3), which encodes a small intraluminal protein that binds cholesterol. Recent work suggests a functional cooperation between NPC1 and NPC2 in releasing the cholesterol from lysosomes. NPC2 is thought to transfer cholesterol to NPC1, which is then hypothesized to transport cholesterol through the glycocalyx to the limiting membrane of late endosomes/early lysosomes.

The BALB/cNctr-Npc1^{m1N}/J (Npc1^{-/-}) mouse strain carries a spontaneous mutation of Npc1 and lacks functional NPC1 protein. This mouse model replicates many aspects of both hepatic and neurological disease observed in NPC patients. Characteristic features include hepatomegaly, unesterified cholesterol accumulation in the liver (with foam cells), and increased plasma alanine aminotransferase (ALT) and aspartate aminotransferase (AST) liver enzymes from five to six weeks of age. Initial neurological symptoms appear around six weeks of age, and consist mainly of progressive tremors and ataxia, reflecting the progressive loss of Purkinje cells in the cerebellum. Death typically occurs around 12 weeks of age.

Despite intensive work, the precise mechanisms responsible for both brain and liver dysfunction are not fully defined. Although oxysterols have been reported to be a potential sensitive and specific biomarker for NPC, identification of additional biomarkers reflecting multiple aspects of the NPC pathological cascade will be of significant value in establishing a diagnosis and investigating candidate therapeutic interventions.

### SUMMARY OF THE INVENTION

As described below, this invention provides novel biomarkers for Niemann-Pick disease, type C (NPC).

In aspects, the invention provides methods for identifying a subject as having NPC. In embodiments, the methods involve detecting the level of a biomarker selected from the group comprising i) galectin-3 (LGALS3); ii) cathepsin D (CTSD); iii) LGALS3 and CTSD; and iv) LGALS3 and/or CTSD in combination with at least one additional NPC associated biomarker in a sample obtained from the subject. In embodiments, the methods involve comparing the level of the biomarker to a reference. In embodiments, the subject is identified as having NPC when the level of the biomarker is increased relative to the reference.

In aspects, the invention provides methods for identifying NPC in a subject. In embodiments, the methods involve detecting the level of a biomarker selected from the group comprising i) galectin-3 (LGALS3); ii) cathepsin D (CTSD); iii) LGALS3 and CTSD; and iv) LGALS3 and/or CTSD in combination with at least one additional NPC associated biomarker in a sample obtained from the subject. In embodiments, the methods involve comparing the level of the biomarker to a reference. In embodiments, NPC is identified in the subject when the level of the biomarker is increased relative to the reference.

In aspects, the invention provides methods for characterizing the stage of neurological disease in a subject. In embodiments, the methods involve detecting the level of a biomarker selected from the group comprising i) galectin-3 (LGALS3); ii) cathepsin D (CTSD); iii) LGALS3 and CTSD; and iv) LGALS3 and/or CTSD in combination with at least one additional NPC associated biomarker in a sample obtained from the subject. In embodiments, the methods involve comparing the level of the biomarker to a reference. In embodiments, an increase in the level of the biomarker relative to the reference identifies the subject as having a later stage of neurological disease. In embodiments, the subject has NPC.

In any of the above aspects and embodiments, the one additional NPC associated biomarker can be a NPC associated protein, NPC associated lipid, or NPC associated oxysterol. In embodiments, the NPC associated protein is calbindin D, fatty acid binding protein 3, or fatty acid binding protein 7. In embodiments, the NPC associated oxysterol is 7-ketocholesterol or 3β,5α,6β-cholestane-triol.

In any of the above aspects and embodiments, the biomarker can be LGALS3. In any of the above aspects and embodiments, the biomarker can be CTSD. In any of the above aspects and embodiments, the biomarker can be LGALS3 and CTSD. In embodiments, the biomarker further comprises calbindin D, fatty acid binding protein 3, fatty acid binding protein 7,7-ketocholesterol, or 3β,5α,6β-cholestane-triol.

In any of the above aspects and embodiments, the level of the biomarker is increased 1.5, 2, 2.5, 3, 3.5, 4, 5, 6, 7, 8, 9, 10, 11, 12, 13, 14, 15-fold or more relative to the reference.

In any of the above aspects and embodiments, the reference is the level of the biomarker in a control.

In aspects, the invention provides methods for identifying a subject as having NPC. In embodiments, the methods involve detecting the level of a biomarker selected from the group comprising galectin-3 (LGALS3) and cathepsin D (CTSD) in a sample obtained from the subject. In embodiments, the subject is identified as having NPC when the level of the LGALS3 biomarker is at least about 10 ng/mL. In embodiments, the subject is identified as having NPC when the level of the LGALS3 biomarker is at least about 12 ng/mL, 12.5 ng/mL, 13 ng/mL, 13.5 ng/mL, 14 ng/mL, 15 ng/mL, 20 ng/mL, 25 ng/mL, or 50 ng/mL. In embodiments, the subject is identified as having NPC when the level of the CTSD biomarker is at least about 25 ng/mL, 30 ng/mL, 35 ng/mL, 40 ng/mL, or 50 ng/mL.

In aspects, the invention provides methods for identifying NPC in a subject. In embodiments, the methods involve detecting the level of a biomarker selected from the group comprising galectin-3 (LGALS3) and cathepsin D (CTSD) in a sample obtained from the subject. In embodiments, the subject is identified as having NPC when the level of the LGALS3 biomarker is at least about 10 ng/mL. In embodiments, the subject is identified as having NPC when the level of the LGALS3 biomarker is at least about 12 ng/mL, 12.5 ng/mL, 13 ng/mL, 13.5 ng/mL, 14 ng/mL, 15 ng/mL, 20 ng/mL, 25 ng/mL, or 50 ng/mL. In embodiments, the subject is identified as having NPC when the level of the CTSD biomarker is at least about 25 ng/mL, 30 ng/mL, 35 ng/mL, 40 ng/mL, or 50 ng/mL.

In aspects, the invention provides methods for monitoring NPC therapy in a subject. In embodiments, the methods involve detecting the level of a biomarker selected from the group comprising i) galectin-3 (LGALS3); ii) cathepsin D (CTSD); iii) LGALS3 and CTSD; and iv) LGALS3 and/or CTSD in combination with at least one additional NPC associated biomarker in a sample obtained from the subject. In embodiments, the methods involve comparing the level of the biomarker to a reference. In embodiments, a therapy that reduces the level of the biomarker is identified as effective. In embodiments, the reference is the level of the biomarker in a control. In related embodiments, the control is a sample obtained from the subject prior to therapy or at an earlier time point during therapy.

In aspects, the invention provides methods for detecting an agent's therapeutic efficacy in a subject having NPC. In embodiments, the methods involve detecting the level of a biomarker selected from the group comprising i) galectin-3 (LGALS3); ii) cathepsin D (CTSD); iii) LGALS3 and CTSD; and iv) LGALS3 and/or CTSD in combination with at least one additional NPC associated biomarker in a sample obtained from the subject. In embodiments, the methods involve comparing the level of the biomarker to a reference. In embodiments, (i) a maintenance or increase in the level indicates that the agent lacks efficacy in the subject, and (ii) a decrease in the level indicates that the agent has therapeutic efficacy in the subject. In embodiments, the reference is the level of the biomarker in a control. In related embodiments, the control is a sample obtained from the subject prior to therapy or at an earlier time point during therapy.

In the above aspects and embodiments, the one additional NPC associated biomarker can be a NPC associated protein, NPC associated lipid, or NPC associated oxysterol. In embodiments, the NPC associated protein is calbindin D, fatty acid binding protein 3, or fatty acid binding protein 7. In embodiments, the NPC associated oxysterol is 7-ketocholesterol or 3β,5α,6β-cholestane-triol.

In any of the above aspects and embodiments, the subject can be human.

In any of the above aspects and embodiments, the sample is a biological fluid selected from the group consisting of blood, blood serum, plasma, cerebrospinal fluid, saliva, and urine. In embodiments, the sample is blood, blood serum, plasma, or cerebrospinal fluid.

In any of the above aspects and embodiments, the level is detected by chromatography, mass spectrometry, spectroscopy, or immunoassay.

In aspects, the invention provides kits for aiding the diagnosis of NPC.

In embodiments, the kit contains at least one reagent capable of detecting or capturing galectin-3 (LGALS3) and/or cathepsin D (CTSD). In embodiments, the reagent is an antibody that specifically binds to LGALS3 and/or CTSD. In embodiments, the kit further contains directions for using the reagent to analyze the level of LGALS3 and/or CTSD. In embodiments, the kit further contains at least one additional reagent capable of detecting or capturing calbindin D, fatty acid binding protein 3, fatty acid binding protein 7,7-ketocholesterol, and/or 3β,5α,6β-cholestane-triol. In related embodiments, the additional reagent is an antibody that specifically binds to calbindin D, fatty acid binding protein 3, fatty acid binding protein 7,7-ketocholesterol, and/or 3β,5α,6β-cholestane-triol.

In embodiments, the kit contains an adsorbent that retains (LGALS3) and/or cathepsin D (CTSD). In embodiments, the kit further contains directions for contacting a test sample with the adsorbent and detecting LGALS3 and/or CTSD retained by the adsorbent. In embodiments, the kit further contains at least one additional adsorbent that retains calbindin D, fatty acid binding protein 3, fatty acid binding protein 7,7-ketocholesterol, and/or 3β,5α,6β-cholestane-triol.

Additional objects and advantages of the invention will be set forth in part in the description which follows, and in part will be obvious from the description, or may be learned by practice of the invention. The objects and advantages of the invention will be realized and attained by means of the elements and combinations disclosed herein, including those pointed out in the appended claims. It is to be understood that both the foregoing general description and the following detailed description are exemplary and explanatory only and are not restrictive of the invention as claimed. The accompanying drawings, which are incorporated in and constitute a part of this specification, illustrate several embodiments of the invention and, together with the description, serve to explain the principles of the invention.

### DEFINITIONS

To facilitate an understanding of the present invention, a number of terms and phrases are defined below.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

A “biomarker” as used herein generally refers to a molecule that is differentially present in a sample taken from a subject of one phenotypic status (e.g., having a disease) as compared with another phenotypic status (e.g., not having the disease). A biomarker is differentially present between different phenotypic statuses if the mean or median level of the biomarker in a first phenotypic status relative to a second phenotypic status is calculated to represent statistically significant differences. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. As such, biomarkers can find use as markers for, for example, disease (diagnostics), therapeutic effectiveness of a drug (theranostics), and of drug toxicity.

As used herein, the term “galectin-3” (LGALS3) refers to a polypeptide having at least 80%, 85%, 90%, 95%, or more amino acid identity to the following sequence:

MADNFSLHDALSGSGNPNPQGWPGAWGNQPAGAGGYPGASYPGAYPGQAP
PGAYPGQAPPGAYPGAPGAYPGAPAPGVYPGPPSGPGAYPSSGQPSATGA
YPATGPYGAPAGPLIVPYNLPLPGGVVPRMLITILGTVKPNANRIALDFQ
RGNDVAFHFNPRFNENNRRVIVCNTKLDNNWGREERQSVFPFESGKPFKI
QVLVEPDHFKVAVNDAHLLQYNEIRVKKLNEISKLGISGDIDLTSASYTMI

As used herein, the term “cathepsin D” (CTSD) refers to a compound having the CAS number 9025-26-7, including a pharmaceutically acceptable salt, solvate, hydrate, geometrical isomer, tautomer, optical isomer, isotopic derivative, polymorph, prodrug, or N-oxide thereof.

By “agent” is meant any small molecule chemical compound, antibody, nucleic acid molecule, or polypeptide, or fragments thereof.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition, e.g., Niemann-Pick disease, type C (NPC).

As used herein, the terms “treat,” treating,” “treatment,” and the like refer to reducing or ameliorating a disease or condition, e.g., NPC, and/or symptoms associated therewith. It will be appreciated that, although not precluded, treating a disease or condition does not require that the disease, condition, or symptoms associated therewith be completely eliminated.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

As used herein, the term “sample” includes a biologic sample such as any tissue, cell, fluid, or other material derived from an organism.

By “reference” is meant a standard of comparison. For example, the galectin-3 and/or cathepsin D level present in a patient sample may be compared to the level of the compound(s) in a corresponding healthy cell or tissue or in a diseased cell or tissue (e.g., a cell or tissue derived from a subject having NPC).

By “periodic” is meant at regular intervals. Periodic patient monitoring includes, for example, a schedule of tests that are administered daily, bi-weekly, bi-monthly, monthly, biannually, or annually.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compounds, compositions, or methods provided herein can be combined with one or more of any of the other compositions and methods provided herein.

### DESCRIPTION OF THE DRAWINGS

FIG. 1 includes a principal component analysis (PCA) plot. The PCA plot shows a view of the 48 Npc1^{-/-} and control samples over the whole time course (1, 3, 5, 7, 9, and 11 weeks). Control mice figure as circles, mutant as diamonds. Age is size-coded with symbol sizes increasing with age (1-week-old samples<3-week-old<5-week-old<7-week-old<9-week-old<11-week-old).

FIG. 2 includes a heat map generated with the list of 5327 genes differentially expressed for at least 2 time points between Npc1^{-/-} and control mice (P-value ≦0.05 and |fold-change|≧1.3).

FIGS. 3A-3R include graphs showing the validation of altered expression of 18 genes by qPCR. The X axis shows the age of mice in weeks. qPCR and microarray data are presented on the same graph for comparison. Control mice samples in white, Npc1^{-/-} mice in black. FIGS. 3A-3E include genes involved in cholesterol homeostasis; FIGS. 3F-G include genes involved in lipid homeostasis; FIGS. 3H-3J include genes involved in cell adhesion and extracellular matrix remodeling; FIGS. 3K-3M include genes involved in immune response and inflammation; FIGS. 3N-3O include genes involved in developmental signaling; and FIGS. 3P-3Q include genes involved in oxidative stress. A Games-Howell test was performed to determine the significance of the difference in means between control and mutant mice at each age: *P-value ≦0.05; **P-value ≦0.001.

FIG. 4 includes a schematic representation of the different categories of pathways with DEG between Npc1^{-/-} and control mice at each age. Thickness of bars increases with the number of significant pathways identified using each gene list (FDR step-up value ≦0.05). Light grey indicates pathways with mostly up-regulated genes (≧75%), black pathways with mostly down-regulated genes (≧75%), and dark grey pathways with both up- and down-regulated genes. The vertical, black, dashed line indicates the onset of pathological symptoms.

FIGS. 5A-5H include graphs showing the increased expression of LGALS3 in NPC disease. FIG. 5A shows expression of Lgals3 in the NPC mouse model over the progression of the disease from the microarrays. Npc1^{+/+} mice in white, Npc1^{-/-} in black (N=4). *P-value ≦0.05; **P-value ≦0.0001. FIG. 5B shows elevation of LGALS3 concentrations in serum of NPC patients (N=30) compared to pediatric controls (N=16), and four lysosomal storage diseases (infantile neuronal ceroid lipofuscinosis, INCL, N=3; Gaucher Disease, GD, N=5; GM-1 gangliosidosis, N=9; and GM-2 gangliosidosis, N=1). Mean and standard deviation figured for each group. Dotted line represents 90% sensitivity as determined by ROC analysis. FIG. 5C shows the results from ROC analysis for LGALS3. FIGS. 5D-5F show the correlation of serum LGALS3 concentrations with AST/ALT levels (FIG. 5D), total bilirubin levels (FIG. 5E), and disease severity rank (1 to 39, from less to most severe case) in NPC patients (FIG. 5F). FIG. 5G-5H show the correlation of LGALS3 levels with 7-ketocholesterol (FIG. 5G) and 3β,5α,6β-cholestane-triol levels (FIG. 5H).

FIGS. 6A-6H include graphs showing the increased expression of CTSD in NPC disease. FIG. 6A shows expression of Ctsd in the NPC mouse model over the progression of the disease from the microarrays. Npc1^{+/+} mice in white, Npc1^{-/-} in black (N=4). *P-value ≦0.05; **P-value ≦0.0001. FIG. 6B shows elevation of CTSD concentrations in serum of NPC patients (N=30) compared to pediatric controls (N=16), and four lysosomal storage diseases (infantile neuronal ceroid lipofuscinosis, INCL, N=3; Gaucher Disease, GD, N=5; GM-1 gangliosidosis, N=9; and GM-2 gangliosidosis, N=1). Mean and standard deviation figured for each group. Dotted line represents the 90% sensitivity determined by the ROC analysis. FIG. 6C shows ROC analysis for CTSD. FIGS. 6D-6F: Correlation of serum CTSD concentrations with AST/ALT levels (FIG. 6D), total bilirubin levels (FIG. 6E), and disease severity rank (1 to 39, from less to most severe case) in NPC patients (FIG. 6F). FIGS. 6G-6H: Correlation of CTSD levels with 7-ketocholesterol (FIG. 6G) and 3β,5α,6β-cholestane-triol levels (FIG. 6H).

FIG. 7A-7F include graphs showing additional information, including the absence of effect of miglustat and the correlation between CTSD and LGALS3 serum concentrations. FIG. 7A shows expression of Plau in the mouse model over the progression of the disease from the microarrays. Npc1^{+/+} mice figure in white, Npc1^{-/-} figure in black (N=4). *p-value ≦0.05; **p-value ≦0.0001. FIGS. 7B and 7C show that there is no significant differences between the concentrations of LGALS3 (FIG. 7B) and CTSD (FIG. 7C) in serum of NPC patient treated or not with miglustat. FIGS. 7D and 7E show the percent change of LGALS3 (FIG. 7D) and CTSD concentrations (FIG. 7E) for 6 patients after miglustat treatment. FIG. 7F shows the correlation between CTSD and LGALS3 concentrations in NPC patients' serum.

### DETAILED DESCRIPTION OF THE INVENTION

This invention is based, at least in part, on the discovery that galectin-3 (LGALS3) and cathepsin D (CTSD) are biomarkers for Niemann-Pick disease, type C (NPC). Accordingly, the invention provides methods and kits that are useful in the diagnosis, treatment, and prevention of NPC. The invention further provides methods and kits for evaluating therapies for treating a patient identified as having NPC.

NPC is a lysosomal storage disorder characterized by liver disease and progressive neurodegeneration. Deficiency in NPC1 or NPC2 lysosomal proteins leads to accumulation of cholesterol and glycosphingolipids in late endosomes and early lysosomes. In order to identify pathological mechanisms underlying NPC and uncover potential biomarkers, liver gene expression changes in a mouse Npc1 model, at six ages spanning the pathological progression of the disease were characterized. Altered gene expression was identified at all ages, including in one-week-old asymptomatic mutant mice. Biological pathways showing early altered expression patterns included: zinc finger protein 202-regulated genes associated with lipid metabolism, cytochrome P450 enzymes involved in arachidonic acid and drug metabolism, inflammation and immune responses, mitogen-activated protein kinase (MAPK) and G-protein signaling, cell cycle regulation, cell adhesion and cytoskeleton remodeling. In contrast, apoptosis and oxidative stress appeared to be predominately late pathological processes. To identify candidate biomarkers useful for diagnosis or monitoring disease progression, differentially expressed genes were screened for known secreted proteins. Among 103 genes with a modified expression for at least four ages, increased serum levels of galectin-3 (LGALS3), a pro-inflammatory molecule, and cathepsin D (CTSD), a lysosomal aspartic protease, were observed in NPC patients. Serum levels of both CTSD and LGALS3 correlated with neurological disease status and were specific for NPC. Therefore, LGALS3 and CTSD have diagnostic value and will also serve as biomarkers in therapeutic trials.

### Diagnostics and Diagnostic Assays

The present invention features diagnostic assays for the detection of Niemann-Pick disease, type C (NPC). In embodiments, the level of a biomarker(s) is measured in a subject sample and used to characterize NPC. In embodiments, the biomarker is galectin-3 (LGALS3) and/or cathepsin D (CTSD). In related embodiments, the biomarker further comprises one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like).

Biological samples include tissue samples (e.g., cell samples, biopsy samples, and the like) and bodily fluids, including, but not limited to, blood, blood serum, plasma, cerebrospinal fluid, saliva, and urine. Samples can optionally be treated to enrich for the biomarker(s) using enrichment and separation methods well known in the art.

Elevated levels of the biomarker(s) are considered a positive indicator of NPC. In general, an increase in the levels of LGALS3 and/or CTSD, optionally in combination with one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like), is indicative of NPC. The increase in biomarker levels may be by at least about 10%, 25%, 50%, 75%, 90% or more. The increase in biomarker levels may be by at least about 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 55, 60, 65, 70, 75, 80, 85, 90, 95-fold or more.

In embodiments, multiple biomarkers are measured, e.g., LGALS3 and/or CTSD, optionally in combination with one or more additional NPC biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like). The use of multiple biomarkers increases the predictive value of the test and provides greater utility in diagnosis, toxicology, patient stratification and patient monitoring. The process called “Pattern recognition” detects the patterns formed by multiple biomarkers greatly improves the sensitivity and specificity of the diagnostic assay for predictive medicine. Subtle variations in data from clinical samples indicate that certain patterns of biomarkers can predict phenotypes such as the presence or absence of a certain disease, a particular stage of progression, or a positive or adverse response to drug treatments.

Detection of an alteration relative to a reference sample (e.g., normal sample) can be used as a diagnostic indicator of NPC.

In embodiments, the invention provides methods for identifying a subject as having or having a propensity to develop NPC.

In related embodiments, the methods involve detecting the level of a biomarker selected from the group comprising LGALS3 and/or CTSD, optionally in combination with one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like), in a sample obtained from the subject. In embodiments, the methods involve comparing the level of the biomarker to a reference. In embodiments, the methods involve identifying the subject as having NPC when the level of the biomarker is increased relative to the reference.

In related embodiments, the methods involve detecting the level of a biomarker selected from the group comprising LGALS3 and/or CTSD, optionally in combination with one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like), in a sample obtained from the subject. In embodiments, the subject is identified as having NPC when the level of the LGALS3 biomarker is at least about 10 ng/mL. In embodiments, the subject is identified as having NPC when the level of the LGALS3 biomarker is at least about 12 ng/mL, 12.5 ng/mL, 13 ng/mL, 13.5 ng/mL, 14 ng/mL, 15 ng/mL, 20 ng/mL, 25 ng/mL, or 50 ng/mL. In embodiments, the subject is identified as having NPC when the level of the CTSD biomarker is at least about 25 ng/mL, 30 ng/mL, 35 ng/mL, 40 ng/mL, or 50 ng/mL.

In embodiments, the invention provides methods for identifying NPC in a subject.

In related embodiments, the methods involve detecting the level of a biomarker selected from the group comprising LGALS3 and/or CTSD, optionally in combination with one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like), in a sample obtained from the subject. In embodiments, the methods involve comparing the level of the biomarker to a reference. In embodiments, the methods involve identifying NPC in the subject when the level of the biomarker is increased relative to the reference.

In related embodiments, the methods involve detecting the level of a biomarker selected from the group comprising LGALS3 and/or CTSD, optionally in combination with one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like), in a sample obtained from the subject. In embodiments, the subject is identified as having NPC when the level of the LGALS3 biomarker is at least about 10 ng/mL. In embodiments, the subject is identified as having NPC when the level of the LGALS3 biomarker is at least about 12 ng/mL, 12.5 ng/mL, 13 ng/mL, 13.5 ng/mL, 14 ng/mL, 15 ng/mL, 20 ng/mL, 25 ng/mL, or 50 ng/mL. In embodiments, the subject is identified as having NPC when the level of the CTSD biomarker is at least about 25 ng/mL, 30 ng/mL, 35 ng/mL, 40 ng/mL, or 50 ng/mL.

In embodiments, the invention provides methods for characterizing the stage of neurological disease in a subject. In related embodiments, the methods involve detecting the level of a biomarker selected from the group comprising LGALS3 and/or CTSD, optionally in combination with one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like), in a sample obtained from the subject. In embodiments, the methods involve comparing the level of the biomarker to a reference. In embodiments, the methods involve identifying the subject as having a later stage of neurological disease when there is an increase in the level of the biomarker relative to the reference. In embodiments, the subject has NPC.

In embodiments, the invention provides methods for monitoring NPC therapy in a subject. In related embodiments, the methods involve detecting the level of a biomarker selected from the group comprising LGALS3 and/or CTSD, optionally in combination with one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like), in a sample obtained from the subject. In embodiments, the methods involve comparing the level of the biomarker to a reference. In embodiments, the methods involve identifying the therapy as effective when there is a decrease in the level of the biomarker relative to the reference.

In embodiments, the invention provides methods for detecting an agent's therapeutic efficacy in a subject having NPC. In related embodiments, the methods involve detecting an alteration in the level of a biomarker selected from the group comprising LGALS3 and/or CTSD, optionally in combination with one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like), in a sample obtained from the subject. In embodiments, the methods involve comparing the level of the biomarker to a reference (e.g., a patient sample taken at an earlier time point or prior to treatment). In embodiments, the methods involve identifying the agent as having therapeutic efficacy in the subject when there is a decrease in the level. In embodiments, the methods involve identifying the agent as lacking therapeutic efficacy in the subject when there maintenance or increase in the level.

In embodiments, the level of the biomarker(s) is measured on at least two different occasions and an alteration in the levels as compared to normal reference levels over time is used as an indicator of NPC. The level of the biomarker(s) in a sample from a subject (e.g., bodily fluids such as blood, blood serum, plasma, cerebrospinal fluid, saliva, and urine) of a subject having NPC or the propensity to develop such a condition may be altered by as little as 10%, 20%, 30%, or 40%, or by as much as 50%, 60%, 70%, 80%, or 90% or more relative to the level of such biomarker(s) in a normal control. In embodiments, a subject sample is collected prior to the onset of symptoms of NPC. In embodiments, a subject sample is collected after the onset of symptoms of NPC. In embodiments, a subject sample is collected while the subject is undergoing treatment for NPC.

The diagnostic methods described herein can be used individually or in combination with any other diagnostic method described herein or well known in the art for a more accurate diagnosis of the presence or severity of NPC.

The diagnostic methods described herein can also be used to monitor and manage NPC.

As indicated above, the invention provides methods for aiding an NPC diagnosis using LGALS3 and/or CTSD, optionally in combination with one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like). These biomarker(s) can be used alone, in combination with other biomarkers in any set, or with entirely different markers in aiding NPC diagnosis. The markers are differentially present in samples of an NPC patient and a normal subject in whom NPC is undetectable. Therefore, detection of one or more of these biomarkers in a person would provide useful information regarding the probability that the person may have NPC or regarding the stage of NPC.

The detection of the biomarker(s) is then correlated with a probable diagnosis of NPC. In embodiments, the detection of the mere presence of a biomarker, without quantifying the amount thereof, is useful and can be correlated with a probable diagnosis of NPC. The measurement of biomarkers may also involve quantifying the markers to correlate the detection of markers with a probable diagnosis of NPC. Thus, if the amount of the biomarkers detected in a subject being tested is different compared to a control amount (e.g., higher than the control), then the subject being tested has a higher probability of having NPC.

The correlation may take into account the amount of the biomarker(s) in the sample compared to a control amount of biomarker(s) (e.g., in normal subjects or in non-NPC subjects such as where NPC is undetectable). A control can be, e.g., the average or median amount of the biomarker(s) present in comparable samples of normal subjects in normal subjects or in non-NPC subjects such as where NPC is undetectable. The control amount is measured under the same or substantially similar experimental conditions as in measuring the test amount. As a result, the control can be employed as a reference standard, where the normal (non-NPC) phenotype is known, and each result can be compared to that standard (e.g., a standardized curve for use), rather than re-running a control.

In some embodiments, the control is derived from the patient and provides a reference level of the patient prior to, during, or after treatment for NPC.

Accordingly, a biomarker profile may be obtained from a subject sample and compared to a reference biomarker profile obtained from a reference population, so that it is possible to classify the subject as belonging to or not belonging to the reference population. The correlation may take into account the presence or absence of the biomarkers in a test sample and the frequency of detection of the same biomarkers in a control. The correlation may take into account both of such factors to facilitate determination of NPC status.

In certain embodiments of the methods of qualifying NPC status, the methods further comprise managing subject treatment based on the status. The invention also provides for such methods where the biomarker(s) are measured again after subject management. In these cases, the methods are used to monitor the status of NPC, e.g., response to NPC treatment, including improvement, maintenance, or progression of the disease.

A biomarker, individually, can be useful in aiding in the determination of NPC status. First, the selected biomarker is detected in a subject sample using well known methods, including, but not limited to, the methods described herein. Then, the result is compared with a control that distinguishes NPC status from non-NPC status. As is well understood in the art, the techniques can be adjusted to increase sensitivity or specificity of the diagnostic assay depending on the preference of the diagnostician.

While an individual biomarker is a useful diagnostic marker, in some instances, a combination of biomarkers provides greater predictive value than single markers alone. The detection of a plurality of biomarkers (or absence thereof, as the case may be) in a sample can increase the percentage of true positive and true negative diagnoses and decrease the percentage of false positive or false negative diagnoses. Thus, in embodiments, methods of the present invention comprise the measurement of more than one biomarker.

### Detection of Biomarkers

Any suitable method can be used to detect the biomarker(s). Successful practice of the invention can be achieved with one or a combination of methods that can detect and, in embodiments, quantify the biomarker(s).

Detection of the biomarkers can be conducted in the same or different samples, the same or separate assays, and may be conducted in the same or different reaction mixtures. Where the biomarkers are assayed in different samples, the samples are usually obtained from the subject during the same procedure (e.g., blood draw, urine collection, tissue extraction, and the like) or with only a relative short time intervening so as to avoid an incorrect result due to passage of time. Where the biomarkers are detected in separate assays, the samples assayed are can be derived from the same or different samples obtained from the subject to be tested.

LGALS3 and/or CTSD, optionally in combination with one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like), can be detected using one or more methods well known in the art, including, without limit, mass spectrometry, chromatography, spectroscopy (e.g., NMR), elemental analysis, conventional chemical methods, immunoassays, and the like.

In embodiments, the biomarker(s) are detected using mass spectrometry. Mass spectrometry-based methods exploit the differences in mass of biomarkers to facilitate detection. Mass spectrometry can be combined with other assays, e.g., resolving the analyte in a sample by one or two passes through liquid or gas chromatography followed by mass spectrometry analysis. Methods for preparing a biological sample for analysis by mass spectrometry are well known in the art. Suitable mass spectrometers for use include, without limit, electrospray ionization mass spectrometry (ESI-MS), ESIMS/MS, ESI-MS/(MS)n (n is an integer greater than zero), matrix-assisted laser desorption ionization time-of-flight mass spectrometry (MALDI-TOF-MS), electron impact ionization mass spectrometry (EI-MS), chemical ionization mass spectrometry (CI-MS), surface-enhanced laser desorption/ionization time-of-flight mass spectrometry (SELDI-TOF-MS), desorption/ionization on silicon (DIOS), secondary ion mass spectrometry (SIMS), quadrupole timeof-flight (Q-TOF), atmospheric pressure chemical ionization mass spectrometry (APCI-MS), APCI-MS/MS, APCI(MS)11, atmospheric pressure photoionization mass spectrometry (APPI-MS), APPI-MS/MS, APPI-(MS), quadrupole, fourier transform mass spectrometry (FTMS), ion trap, and hybrids of these methods, e.g., electrospray ionization quadrupole time-of-flight mass spectrometry (UPLC-ESI-QTOFMS) and two-dimensional gas chromatography electron impact ionization mass spectrometry (GCxGC-EI-MS).

The methods may be performed in an automated (Villanueva, et al., Nature Protocols (2006) 1(2):880-891) or semi-automated format. This can be accomplished, for example with MS operably linked to a liquid chromatography device (LC-MS/MS or LC-MS) or gas chromatography device (GC-MS or GC-MS/MS). Methods for performing MS are known in the field and have been disclosed, for example, in US Patent Application Publication Nos: 20050023454 and 20050035286; U.S. Pat. No. 5,800,979; and the references disclosed therein.

Samples are collected on a collection layer. They may then be analyzed by a spectroscopic method based on matrix-assisted laser desorption/ionization (MALDI), electrospray ionization (ESI), and the like.

Other techniques for improving the mass accuracy and sensitivity of the MALDI-TOF MS can be used to analyze the analytes obtained on the collection membrane. These include the use of delayed ion extraction, energy reflectors and ion-trap modules. In addition, post source decay and MS-MS analysis are useful to provide further structural analysis. With ESI, the sample is in the liquid phase and the analysis can be by ion-trap, TOF, single quadrupole or multi-quadrupole mass spectrometers. The use of such devices (other than a single quadrupole) allows MS-MS or MSⁿ analysis to be performed. Tandem mass spectrometry allows multiple reactions to be monitored at the same time.

Capillary infusion may be employed to introduce the marker to a desired MS implementation, for instance, because it can efficiently introduce small quantities of a sample into a mass spectrometer without destroying the vacuum. Capillary columns are routinely used to interface the ionization source of a MS with other separation techniques including gas chromatography (GC) and liquid chromatography (LC). GC and LC can serve to separate a solution into its different components prior to mass analysis. Such techniques are readily combined with MS, for instance. One variation of the technique is that high performance liquid chromatography (HPLC) can now be directly coupled to mass spectrometer for integrated sample separation/and mass spectrometer analysis.

Quadrupole mass analyzers may also be employed as needed to practice the invention. Fourier-transform ion cyclotron resonance (FTMS) can also be used for some invention embodiments. It offers high resolution and the ability of tandem MS experiments. FTMS is based on the principle of a charged particle orbiting in the presence of a magnetic field. Coupled to ESI and MALDI, FTMS offers high accuracy with errors as low as 0.001%.

In embodiments, the diagnostic methods of the invention may further comprise identifying significant peaks from combined spectra. The methods may also further comprise searching for outlier spectra. In other embodiments, the methods of the invention further comprise determining distant dependent K-nearest neighbors.

In embodiments, an ion mobility spectrometer can be used to detect and characterize the biomarker(s). The principle of ion mobility spectrometry is based on different mobility of ions. Specifically, ions of a sample produced by ionization move at different rates, due to their difference in, e.g., mass, charge, or shape, through a tube under the influence of an electric field. The ions (typically in the form of a current) are registered at the detector which can then be used to identify a biomarker or other substances in a sample. One advantage of ion mobility spectrometry is that it can operate at atmospheric pressure.

In embodiments, the procedure is electrospray ionization quadrupole mass spectrometry with time of flight (TOF) analysis, known as UPLC-ESI-QTOFMS.

In embodiments, detection of the biomarker(s) involves chemical methods well known in the art. In embodiments, the chemical method is chemical extraction. In embodiments, the chemical method is chemical derivitization.

In embodiments, detection of the biomarker(s) involves use of chromatography methods that are well known in the art. Such chromatography methods include, without limit, column chromatography, ion exchange chromatography, hydrophobic (reverse phase) liquid chromatography, or other chromatography, such as thinlayer, gas, or liquid chromatography (e.g., high-performance liquid chromatography), or any combination thereof.

In embodiments, detection of the biomarker(s) involves use of spectroscopy methods that are well known in the art. Such chromatography methods include, without limit, NMR, IR, and the like.

In embodiments, detection of the biomarker(s) involves elemental analysis methods that are well known in the art. Such elemental analysis methods include, without limit, combustion analysis, gravimetry, atomic spectroscopy, and the like.

In embodiments, detection of the biomarker(s) involves use of immunoassays. In embodiments, the immunoassays involve the use of antibodies. Suitable immunoassays include, without limit, ELISA, flow chamber adhesion, colorimetric assays (e.g., antibody based colorimetric assays), biochip (e.g., antibody based biochip), and the like.

Analytes (e.g., biomarkers) can be detected by a variety of detection methods selected from, for example, a gas phase ion spectrometry method, an optical method, an electrochemical method, atomic force microscopy and a radio frequency method. In one embodiment, mass spectrometry, e.g., SELDI, is used. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

Other variations of the assays described herein to provide for different assay formats for detection of the biomarker(s) will be readily apparent to the one of ordinary skill in the art upon reading the present disclosure.

### Diagnostic Kits

The invention provides kits for diagnosing or monitoring NPC, or for selecting a treatment for NPC.

In embodiments, the kits include one or more reagents capable of detecting and/or capturing LGALS3 and/or CTSD, optionally in combination with one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like). In related embodiments, the reagent is an antibody or a mass spectrometry probe.

In embodiments, the kits include an adsorbent that retains LGALS3 and/or CTSD, optionally in combination with one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like). In related embodiments, the kits further contain directions for contacting a test sample with the adsorbent and detecting LGALS3 and/or CTSD, optionally in combination with one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like), retained by the adsorbent.

In embodiments, the reagents and/or adsorbents are provided on a solid support (e.g., chip, microtiter plate, bead, resin, and the like).

In embodiments, the kits include washing solution(s) or instructions for making a washing solution, in which the combination of the reagent/adsorbent and the washing solution allows capture of the biomarkers on the reagent/adsorbent.

In embodiments, the kits include LGALS3 and/or CTSD, and optionally one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like), which can be used as standard(s) for calibration as may be desired.

In embodiments, the kit contains a container(s) that houses the components of the kit (e.g., reagent, adsorbant, solid support, and the like). Such containers can be boxes, ampoules, bottles, vials, tubes, bags, pouches, blister-packs, or other suitable container forms known in the art. Such containers can be made of plastic, glass, laminated paper, metal foil, and the like.

In embodiments, the kits further contain directions for using the kit in any of the methods described herein (e.g., diagnosing NPC, monitoring NPC, characterizing NPC, selecting a treatment for NPC, and the like). In embodiments, the instructions include at least one of the following: description of the reagents, supports, and/or adsorbents; warnings; indications; counter-indications; animal study data; clinical study data; and/or references. The instructions may be printed directly on the container (when present), or as a label applied to the container, or as a separate sheet, pamphlet, card, or folder supplied in or with the container.

### Subject Monitoring

The disease state or treatment of a subject having NPC can be monitored using the methods and biomarkers of the invention. In embodiments, methods and biomarkers of the invention are used by a clinician to identify subjects as having or not having NPC. For example, a general practitioner may use the methods delineated herein to screen patients for the presence of NPC. In embodiments, the expression of biomarker(s) present in a patient sample, e.g., bodily fluid such as blood, blood serum, plasma, cerebrospinal fluid, saliva, and urine, is monitored. Such monitoring may be useful, for example, in assessing the efficacy of a particular drug in a subject or in assessing disease progression. Therapeutics that decrease the expression of a biomarker of the invention (e.g., LGALS3 and/or CTSD, optionally in combination with one or more additional NPC associated biomarkers, e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like) are taken as particularly useful in the invention.

In embodiments, the biomarker(s) are monitored prior to administering therapy. These results provide a baseline that describes the level of the biomarker(s) prior to treatment.

In embodiments, the biomarker(s) are monitored periodically. In embodiments, the biomarker(s) are monitored periodically throughout treatment. A therapy is identified as efficacious when a diagnostic assay of the invention detects a decrease in marker levels relative to the baseline level of marker prior to treatment.

### Types of Biological Samples

The level of LGALS3 and/or CTSD, and optionally one or more additional NPC associated biomarkers (e.g., NPC associated proteins, such as Calbindin D, Fatty Acid Binding Protein 3 or 7, NPC associated lipids, NPC associated oxysterols, such as 7-ketocholesterol and/or 3β,5α,6β-cholestane-triol, and the like), is measured in different types of samples. In embodiments, the level of the biomarker(s) is measured in a biologic sample. Suitable biologic samples include, without limit, a tissue sample (e.g., from a biopsy) and biological fluids (e.g., blood, blood serum, plasma, cerebrospinal fluid, saliva, urine, or any other biological fluid useful in the methods of the invention). In embodiments, the sample is a urine sample derived from the patient.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Identification and Validation of Differentially Expressed Genes

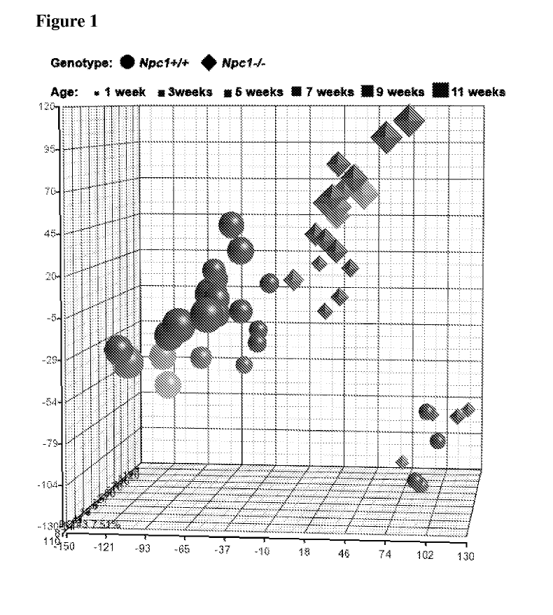

To identify differentially expressed genes occurring in Npc1^{-/-} liver tissue, microarray analysis was performed using cDNA isolated from female control (Npc1+/+) or mutant (Npc1^{-/-}) mice, of one, three, five, seven, nine, and eleven weeks of age. Principal component analysis (PCA) identified three distinct expression groups: 1) one-week-old control and mutant mice, 2) three- to eleven-week-old Npc1^{-/-} mice, and 3) three- to eleven-week-old control mice (FIG. 1). This delineation of specific groups demonstrates that differential gene expression in Npc1 mutant mice occurs by three weeks of age, prior to onset of symptoms.

To identify differentially expressed genes (DEG), the array results from control and mutant mice at each age were compared. Selection criteria were based on combined P-value (P≦0.05) and fold-change (FC ≦−1.3 or FC ≧1.3). Although it varied based on the specific age, in general a slightly higher proportion of DEG showed increased expression in the mutant tissue (ranging from 49% to 65%, Table 1).

TABLE 1
Number of differentially expressed genes and significantly modified
pathways between Npc1^{-/-} and control mice at each age.

| | Number of genes | Number of significant |
|---|---|---|
| | (P-value ≦ 0.05 and | pathway maps (FDR step-up |
| Age | \| fold-change \| ≧ 1.3)* | value ≦ 0.05 in GeneGo)** |
| 1 week | 1319 (753; 57%) | 9 (59%) |
| 3 weeks | 2618 (1461; 56%) | 10 (69%) |
| 5 weeks | 2487 (1626; 65%) | 71 (90%) |
| 7 weeks | 3507 (2162; 62%) | 117 (87%) |
| 9 weeks | 5783 (3145; 54%) | 157 (77%) |
| 11 weeks | 6035 (2973; 49%) | 119 (59%) |
| Total | 11290 | 222 |

*Number and percentage of upregulated genes are given between parentheses.
**Percentage of pathways containing more than 75% of upregulated genes is given between parentheses.

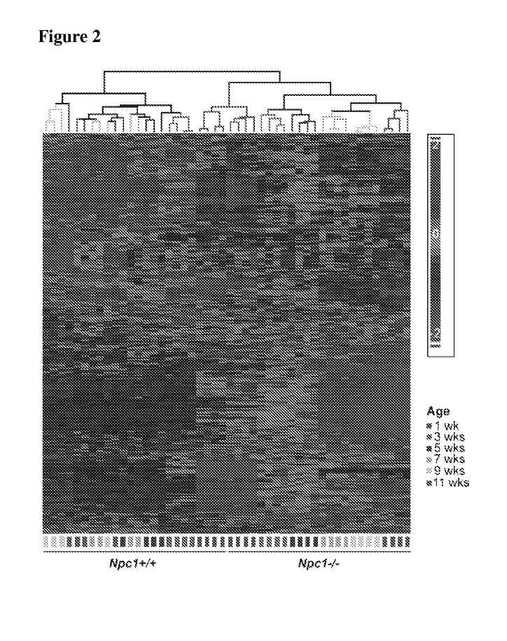

A subset of 5327 DEG that showed altered expression at two or more ages within the study was selected for use in hierarchical clustering of all samples (FIG. 2). The majority of these genes (91%; 4850/5327) showed concordant deregulation of expression over the time course, and for many of these genes the fold-change increased with disease progression (FIG. 2). The hierarchical clustering clearly shows that five- to eleven-week-old samples from mutant mice fell into distinct clusters by age, whereas the control mice did not form distinct clusters by age after three weeks old (FIG. 2). This observation likely reflects the progressive nature of NPC disease compared to control mice, whose gene expression is more constant within these close adult ages. Although gene expression at one week of age is more similar between the two genotype groups than at later time points (PCA analysis, FIG. 1), clustering analysis clearly separated one-week-old Npc1^{+/+} and Npc1^{-/-} mice (FIG. 2).

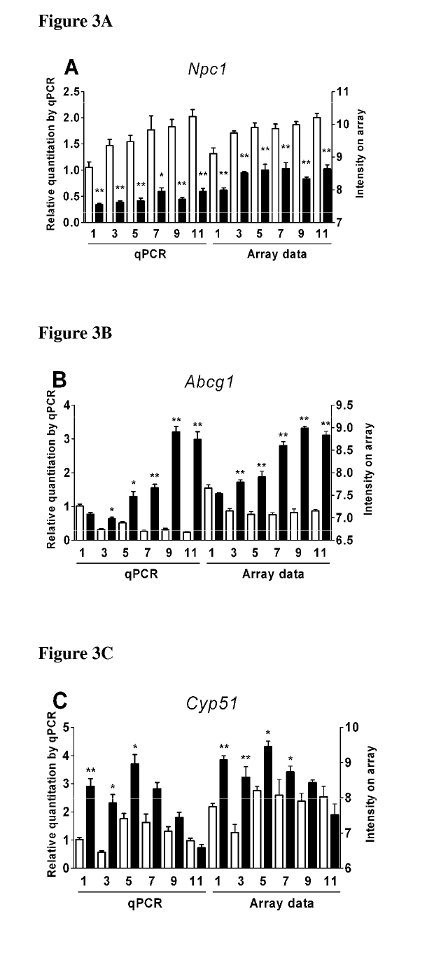

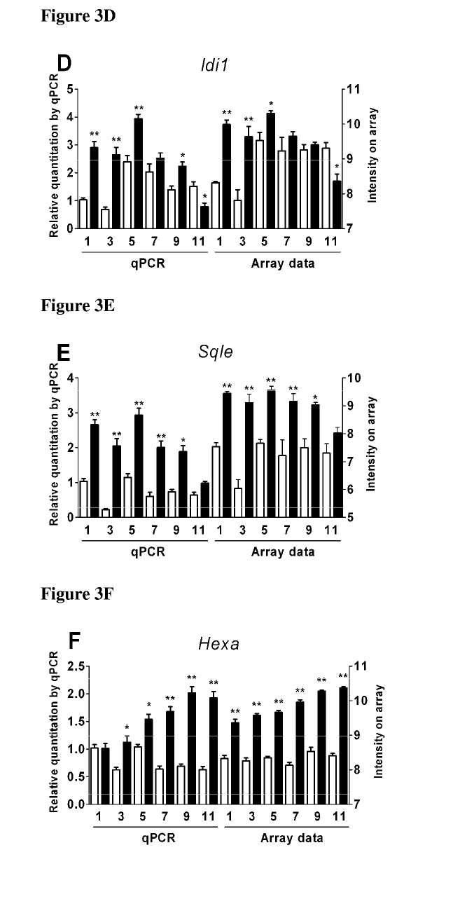

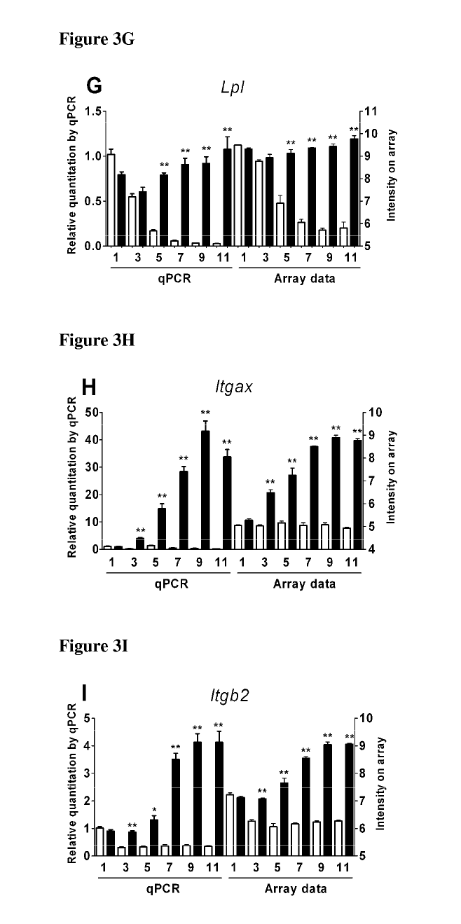

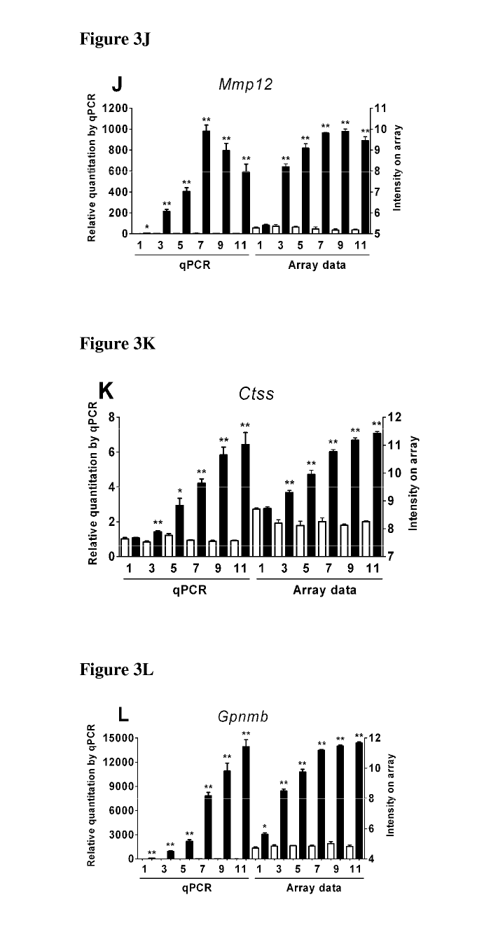

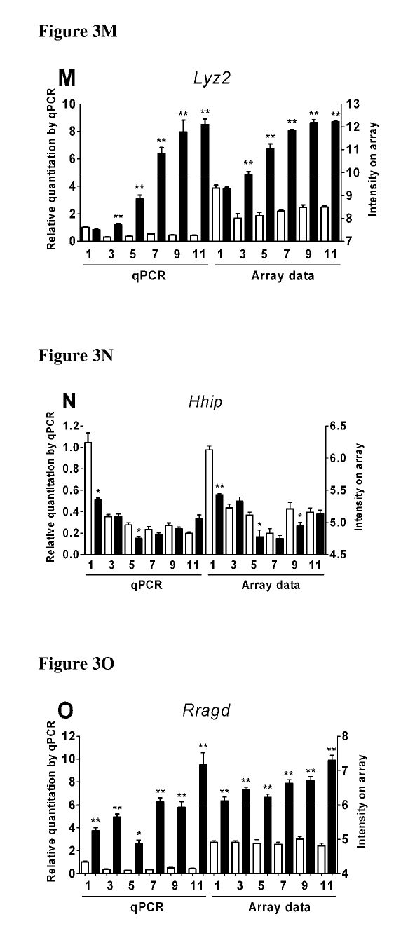

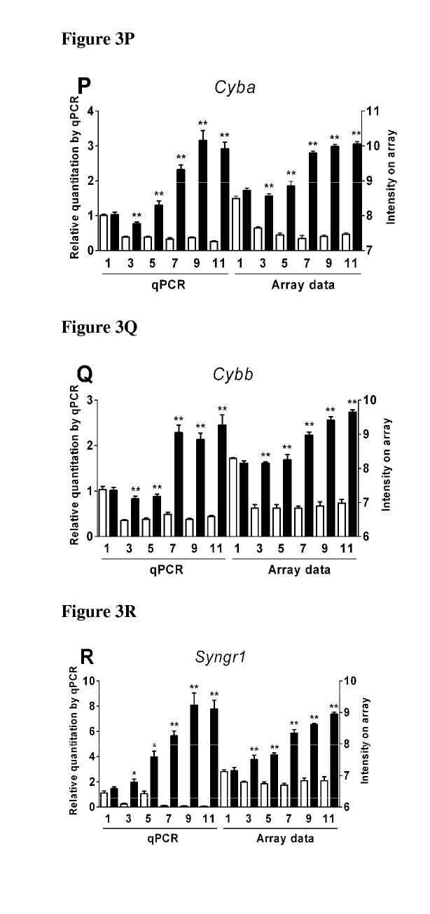

To validate the expression array analysis, qPCR was performed on 18 genes representing different functional categories (cholesterol transport and metabolism, cell adhesion, extracellular matrix remodeling, developmental signaling, oxidative stress and immune response). The results confirmed concordant altered expression of all 18 genes (FIG. 3). Two of these genes, Npc1, as expected, and Hhip (Hedgehog-interacting protein) showed decreased expression in the mutant mice. Sixteen genes, Ctss (cathepsin S), Cyba (cytochrome B245 alpha subunit), Cybb (cytochrome B245 beta subunit), Itgax (integrin alpha x), Itgb2 (integrin beta 2), Mmp12 (matrix metalloproteinase 12), Gpnmb (glycoprotein nmb), Lpl (lipoprotein lipase), Syngr1 (synaptogyrin 1), Hexa (hexosaminidase A), Rragd (Ras-related GTP binding protein D), Lyz2 (lysozyme 2), Cyp51 (cytochrome P450 family 51), Idi1 (idopentenyl-diphosphate delta isomerase), Sqle (squalene epoxidase) and Abcg1 (ATP-binding cassette subfamily G member 1) showed increased expression in Npc1 mutant mice compared to control littermates. When comparing all time points for all 18 genes, the expression array and qPCR data only differed for 4/108 (3.7%) samples (seven-week time point for Cyp51 gene, one-week time point for Hexa gene, nine-week time point for Hhip gene, and nine-week time point for Idi1 gene). At these few points, one method did not identify a modification between control and mutant mice, whereas the second technique detected a significant change in gene expression.

In order to further increase confidence in the expression array data, the expression of genes previously reported to have altered expression in NPC disease was analyzed. As expected, Npc2 and Plin3 genes (perilipin 3 or mannose-6-phosphate receptor-binding protein 1, M6prbp1) were upregulated in mutant tissue, with a fold-change between 1.3 and 1.75 for Npc2, and 1.6 fold to 2.1 for Plin3 (Klein et al., Hepatology 43:126-33 (2006); Blom et al., Hum. Mol. Genet. 12: 257-72 (2003); and Reddy et al., PLoS One 1:e19 (2006)). The results were also compared with the DEG lists from three previous microarray studies conducted using human NPC1 fibroblasts or Npc1 mouse cerebella (Table 2) (Reddy et al., PLoS One 1:e19 (2006); Liao et al., Brain Res. 1325:128-40 (2010); and De Windt et al., DNA Cell Biol. 26:665-71 (2007)).

TABLE 2
Comparison of differentially expressed genes with other microarray studies.

| Species and | | | Number of | Number of | Number of | |
|---|---|---|---|---|---|---|
| cell type | Chip type | Selection criteria | modified genes | murine genes | common DEG | Reference |
| Human | Human cDNA microarrays | Fold-change ≧2 | 1550 | 1010 | 393, 39% | 1 |
| fibroblasts | from Stanford University | and FDR ≦2% | | | (222, 56%) | |
| Human | Illumina (Sentrix Human-8 | Fold-change | 66 | 45 | 18, 40% | 2 |
| fibroblasts | Expression BeadChips) | ≧3.5 | | | (8, 44%) | |
| Murine 3- | Agilent (Mouse Genome | p-value ≦0.05 | 154 | 154 | 75, 49% | 3 |
| week-old | microarrays) and Illumina | and | | | (56, 75%) | |
| cerebella | (MouseWG-6 Bead-Chip) | fold-change ≧1.3 | | | | |

The number of DEG in the initial study, as well as the number of murine orthologs corresponding to these DEG figured in the table. The number and percentage of these DEG in common with the study figured in the next column. The number of these common DEG with altered expression in the same direction in both studies figured in the same column, between parentheses.
¹Reddy et al., PLoS One 1: e19 (2006).
²De Windt et al., DNA Cell Biol. 26: 665-71 (2007).
³Liao et al., Brain Res. 1325: 128-40 (2010).

In order to compare the gene lists with the microarray data on human cells, the human gene symbols were converted into their murine homologs using the Ensembl Biomart tool. About sixty-five percent of the human genes had an identified murine homolog. Forty percent of the DEG published in the two studies using human fibroblasts ( 393/1010, and 18/45) were also present in the DEG list, and about half of these genes had a deregulation of expression in the same direction ( 222/393, and 8/18). Half of the genes with an altered expression in the mouse three-week-old cerebella study ( 75/154) were also deregulated in the liver data, with the majority ( 56/75, 75%) being altered in the same direction.

### Example 2

### Pathway Analysis

To gain insight into pathological processes, the DEG data were used to identify pathways with significantly altered gene expression. MetaCore software was used to identify GeneGo pathway maps containing genes with a modified expression.

TABLE 3
List of all significantly modified pathways (FDR step-up value≦0.05). The number
of DEG at each age figure in the table, with the number of upregulated DEG between
parenthesis, and compared to the total number of genes in the GeneGo pathway map. The
total number of modified pathways is indicated at the end of each category, with the number
of pathways containing more than 75% of upregulated DEG between brackets.

| | 1 week | 3 weeks | 5 weeks | 7 weeks | 9 weeks | 11 weeks |
|---|---|---|---|---|---|---|
| Metabolism | | | | | | |
| Arachidonic acid production | | | 12(8)/50 | | | |
| Atherosclerosis_Role of ZNF202 in regulation of | | 6(4)/21 | 6(6)/21 | 7(6)/21 | 11(6)/21 | 11(6)/21 |
| expression of genes involved in atherosclerosis | | | | | | |
| Butanoate metabolism | 7(2)/65 | | | | | 23(2)/65 |
| Cholesterol biosynthesis | 17(16)/88 | | 15(15)/88 | | | |
| Leucine, isoleucine and valine metabolism.p.2 | | | | 19(3)/78 | | 23(1)/78 |
| Leucine, isoleucine and valine metabolism/Rodent | | | | 21(3)/80 | | 25(1)/80 |
| version | | | | | | |
| Linoleic acid/Rodent version | 12(1)/49 | 16(3)/49 | | 15(2)/49 | 15(2)/49 | 18(3)/49 |
| Mitochondrial ketone bodies biosynthesis and | | | | | | 12(1)/27 |
| metabolism | | | | | | |
| Oxidative phosphorylation | | | | | 30(3)/105 | |
| Pyruvate metabolism/Rodent version | | | | | | 23(5)/66 |
| Regulation of lipid metabolism_FXR-dependent | | | | | | 11(1)/31 |
| negative-feedback regulation of bile acids | | | | | | |
| concentration | | | | | | |
| Regulation of lipid metabolism_PPAR regulation | 8(3)/41 | | | | | |
| of lipid metabolism | | | | | | |
| Regulation of lipid metabolism_Regulation of fatty | | | | | | 9(3)/19 |
| acid synthase activity in hepatocytes | | | | | | |
| Regulation of lipid metabolism_Regulation of lipid | 5(3)/38 | | 9(8)/38 | 10(7)/38 | | 15(4)/38 |
| metabolism via LXR, NF-Y, and SREBP | | | | | | |
| Regulation of lipid metabolism_RXR-dependent | | | 9(3)/30 | | 10(2)/30 | |
| regulation of lipid metabolism via PPAR, RAR | | | | | | |
| and VDR | | | | | | |
| Regulation of lipid metabolism_Stimulation of | | | | | 25(21)/72 | |
| Arachidonic acid production by ACM receptors | | | | | | |
| Regulation of metabolism_Bile acids regulation of | | | 11(7)/37 | | 12(3)/37 | 22(4)/37 |
| glucose and lipid metabolism via FXR | | | | | | |
| Total number of metabolism-related modified | 5 (1) | 2 (0) | 6 (3) | 5 (1) | 6 (1) | 11 (0) |
| pathways | | | | | | |
| Immune response and inflammation | | | | | | |
| Bacterial infections in CF airways | | | 11(10)/58 | 17(12)/58 | | |
| Immune response_CCR3 signaling in eosinophils | | | 16(14)/77 | 29(26)/77 | 34(30)/77 | 34(30)/77 |
| Immune response_Immunological synapse | | | 19(17)/59 | 24(17)/59 | 37(31)/59 | 35(32)/59 |
| formation | | | | | | |
| Immune response_Sialic-acid receptors (Siglecs) | | | | 5(5)/12 | | |
| signaling | | | | | | |
| Immune response_Alternative complement | 6(2)/39 | | | | | 17(6)/39 |
| pathway | | | | | | |
| Immune response_Antigen presentation by MHC | | | 8(8)/12 | 9(9)/12 | 9(9)/12 | 9(9)/12 |
| class II | | | | | | |
| Immune response_Antiviral actions of Interferons | | | | | 18(17)/52 | |
| Immune response_BCR pathway | | | | 19(16)/54 | 22(18)/54 | 19(15)/54 |
| Immune response_CD16 signaling in NK cells | | | | 25(24)/69 | 27(24)/69 | 29(23)/69 |
| Immune response_Classical complement pathway | | | | 15(8)/52 | 16(9)/52 | 26(9)/52 |
| Immune response_CXCR4 signaling via second | | | | 15(11)/34 | 17(12)/34 | 20(14)/34 |
| messenger | | | | | | |
| Immune response_Delta-type opioid receptor | | | 8(8)/29 | 13(12)/29 | 15(13)/29 | 18(16)/29 |
| signaling in T-cells | | | | | | |
| Immune response_Fc epsilon RI pathway | | | | | 19(17)/55 | 21(18)/55 |
| Immune response_Fc gamma R-mediated | | | | 21(21)/45 | 20(18)/45 | |
| phagocytosis in macrophages | | | | | | |
| Immune response_Histamine signaling in dendritic | | | | | 19(12)/50 | |
| cells | | | | | | |
| Immune response_ICOS pathway in T-helper cell | | | | 19(17)/46 | 23(18)/46 | 24(20)/46 |
| Immune response_IFN gamma signaling pathway | | | | 18(17)/54 | 24(20)/54 | |
| Immune response_IL-1 signaling pathway | 6(3)/44 | | | 12(7)/44 | 12(6)/44 | |
| Immune response_IL-10 signaling pathway | | | | 8(7)/26 | | |
| Immune response_IL-15 signaling | | | | | 18(13)/64 | 20(15)/64 |
| Immune response_IL-2 activation and signaling | | | | | 14(7)/49 | |
| pathway | | | | | | |
| Immune response_IL-22 signaling pathway | | | | 15(15)/33 | | |
| Immune response_IL-5 signalling | | | | 15(14)/44 | 14(12)/44 | |
| Immune response_Inhibitory action of lipoxins on | | | | 16(15)/49 | 18(16)/49 | 19(18)/49 |
| superoxide production induced by IL-8 and | | | | | | |
| Leukotriene B4 in neutrophils | | | | | | |
| Immune response_Innate immune response to | | | | | 12(9)/28 | |
| RNA viral infection | | | | | | |
| Immune response_Lectin induced complement | | | | 12(5)/49 | 13(6)/49 | 21(6)/49 |
| pathway | | | | | | |
| Immune response_MIF - the neuroendocrine- | | | 16(16)/46 | 23(21)/46 | 24(21)/46 | |
| macrophage connector | | | | | | |
| Immune response_MIF in innate immunity | | | 9(8)/40 | | | |
| response | | | | | | |
| Immune response_MIF-mediated glucocorticoid | | | 6(6)/22 | | | |
| regulation | | | | | | |
| Immune response_Murine NKG2D signaling | | | | 11(10)/42 | | 16(12)/42 |
| Immune response_NFAT in immune response | | | | 19(17)/51 | 20(17)/51 | 22(18)/51 |
| Immune response_Oncostatin M signaling via | | | 7(5)/37 | | 9(6)/37 | |
| MAPK in human cells | | | | | | |
| Immune response_Oncostatin M signaling via | | | 6(5)/35 | | | |
| MAPK in mouse cells | | | | | | |
| Immune response_PIP3 signaling in B | | | | 19(17)/42 | 19(16)/42 | 23(20)/42 |
| lymphocytes | | | | | | |
| Immune response_Role of DAP12 receptors in NK | | | | | 18(16)/54 | |
| cells | | | | | | |
| Immune response_Role of integrins in NK cells | | | | | 14(14)/38 | |
| cytotoxicity | | | | | | |
| Immune response_Role of the Membrane attack | | | | 12(7)/34 | 14(9)/34 | 19(11)/34 |
| complex in cell survival | | | | | | |
| Immune response_Signaling pathway mediated by | | | | | 11(6)/27 | |
| IL-6 and IL-1 | | | | | | |
| Immune response_T cell receptor signaling | | | | | 19(15)/52 | 20(16)/52 |
| pathway | | | | | | |
| Immune response_TCR and CD28 co-stimulation | | | | 16(14)/40 | 17(12)/40 | 21(16)/40 |
| in activation of NF-kB | | | | | | |
| Immune response_Th17 cell differentiation | | | | | 16(13)/31 | |
| Immune response_TLR signaling pathways | | | 14(14)/56 | | 19(17)/56 | |
| Immune response_TREM1 signaling pathway | | | | 15(12)/56 | 18(13)/56 | 21(16)/56 |
| Inhibitory action of Lipoxin A4 on PDGF, EGF | | | | | 11(8)/34 | |
| and LTD4 signaling | | | | | | |
| Inhibitory action of Lipoxins on neutrophil | | | | 26(25)/57 | 29(27)/57 | 34(31)/57 |
| migration | | | | | | |
| Inhibitory action of Lipoxins on Superoxide | | | | 16(15)/49 | 18(16)/49 | 19(18)/49 |
| production in neutrophils | | | | | | |
| Total number of inflammation-related modified | 2 (0) | 0 | 11 (10) | 28 (21) | 37 (24) | 23 (18) |
| pathways | | | | | | |
| Cytoskeleton remodeling | | | | | | |
| Cytoskeleton remodeling_Cytoskeleton | | | 23(22)/102 | 35(32)/102 | 37(34)/102 | 46(36)/102 |
| remodeling | | | | | | |
| Cytoskeleton remodeling_ESR1 action on | | | 6(3)/20 | 8(7)/20 | 8(5)/20 | 11(7)/20 |
| cytoskeleton remodeling and cell migration | | | | | | |
| Cytoskeleton remodeling_Fibronectin-binding | | | | | 13(13)/31 | 17(15)/31 |
| integrins in cell motility | | | | | | |
| Cytoskeleton remodeling_Integrin outside-in | | | | 22(22)/49 | 25(24)/49 | 27(23)/49 |
| signaling | | | | | | |
| Cytoskeleton remodeling_Keratin filaments | | 11(11)/36 | 14(14)/36 | 14(14)/36 | 13(10)/36 | |
| Cytoskeleton remodeling_Regulation of actin | 11(11)/23 | | | 12(11)/23 | 14(14)/23 | 14(13)/23 |
| cytoskeleton by RhoGTPases | | | | | | |
| Cytoskeleton remodeling_Reverse signaling by | | | 16(15)/31 | 20(19)/31 | 24(19)/31 | 22(18)/31 |
| ephrin B | | | | | | |
| Cytoskeleton remodeling_Role of PDGFs in cell | | | 9(7)/24 | 7(7)/24 | | 9(8)/24 |
| migration | | | | | | |
| Cytoskeleton remodeling_Role of PKA in | | | | | 19(16)/40 | |
| cytoskeleton reorganisation | | | | | | |
| Cytoskeleton remodeling_TGF, WNT and | | | 19(18)/111 | 31(27)/111 | 39(30)/111 | 44(32)/111 |
| cytoskeletal remodeling | | | | | | |
| Total number of cytoskeleton remodeling- | 1 (1) | 1 (1) | 6 (5) | 8 (8) | 9 (8) | 8 (6) |
| related modified pathways | | | | | | |
| Cell adhesion | | | | | | |
| Cell adhesion_Alpha-4 integrins in cell migration | | | | 13(13)/34 | 13(12)/34 | 15(13)/34 |
| and adhesion | | | | | | |
| Cell adhesion_Cadherin-mediated cell adhesion | | | | 10(8)/26 | 12(10)/26 | 14(10)/26 |
| Cell adhesion_Chemokines and adhesion | | | 26(25)/100 | 40(38)/100 | 45(41)/100 | 49(41)/100 |
| Cell adhesion_ECM remodeling | | 16(13)/52 | 18(15)/52 | 23(18)/52 | 25(18)/52 | 28(20)/52 |
| Cell adhesion_Endothelial cell contacts by | | | 7(7)/26 | | | |
| junctional mechanisms | | | | | | |
| Cell adhesion_Ephrin signaling | | | | | 13(9)/45 | |
| Cell adhesion_Gap junctions | | | 12(11)/30 | | | |
| Cell adhesion_Histamine H1 receptor signaling in | | | | 18(16)/45 | 20(18)/45 | 20(18)/45 |
| the interruption of cell barrier integrity | | | | | | |
| Cell adhesion_Integrin inside-out signaling | | | 15(14)/56 | 24(21)/56 | 30(25)/56 | 36(28)/56 |
| Cell adhesion_Integrin-mediated cell adhesion and | | | | | 20(20)/48 | 22(19)/48 |
| migration | | | | | | |
| Cell adhesion_Plasmin signaling | | | | | 20(8)/35 | |
| Cell adhesion_PLAU signaling | | | | | 14(12)/39 | 16(10)/39 |
| Cell adhesion_Role of tetraspanins in the integrin- | | | | 17(17)/37 | 18(17)/37 | 21(20)/37 |
| mediated cell adhesion | | | | | | |
| Total number of cell adhesion-related modified | 0 | 1 (1) | 5 (5) | 7 (7) | 11 (8) | 9 (6) |
| pathways | | | | | | |
| Cell cycle | | | | | | |
| Cell cycle_Cell cycle (generic schema) | | | 8(8)/21 | | | |
| Cell cycle_Chromosome condensation in | | 11(10)/21 | 14(14)/21 | | 10(9)/21 | |
| prometaphase | | | | | | |
| Cell cycle_ESR1 regulation of G1/S transition | | | 8(7)/33 | 10(9)/33 | 14(10)/33 | |
| Cell cycle_Influence of Ras and Rho proteins on | | | | | 19(15)/53 | 19(13)/53 |
| G1/S Transition | | | | | | |
| Cell cycle_Nucleocytoplasmic transport of | | | 7(7)/14 | | 6(5)/14 | |
| CDK/Cyclins | | | | | | |
| Cell cycle_Regulation of G1/S transition (part 1) | | | | 15(15)/38 | 18(15)/38 | 17(14)/38 |
| Cell cycle_Regulation of G1/S transition (part 2) | | | 7(7)/26 | 8(7)/26 | 14(11)/26 | 11(9)/26 |
| Cell cycle_Role of APC in cell cycle regulation | | | 17(17)/32 | | | |
| Cell cycle_Role of Nek in cell cycle regulation | | | 16(15)/32 | | | |
| Cell cycle_Role of SCF complex in cell cycle | | | | | 15(10)/29 | |
| regulation | | | | | | |
| Cell cycle_Sister chromatid cohesion | | | 8(8)/22 | | | |
| Cell cycle_Spindle assembly and chromosome | | | 21(21)/33 | | | |
| separation | | | | | | |
| Cell cycle_Start of DNA replication in early S | | | | 11(10)/32 | 19(14)/32 | |
| phase | | | | | | |
| Cell cycle_The metaphase checkpoint | | | 13(13)/36 | | 13(10)/36 | |
| Cell cycle_Transition and termination of DNA | | 12(9)/28 | 8(8)/28 | | 19(17)/28 | |
| replication | | | | | | |
| Total number of cell cycle-related modified | 0 | 2 (2) | 11 (11) | 4 (4) | 10 (7) | 3 (2) |
| pathways | | | | | | |
| Developmental signaling pathways | | | | | | |
| Development_A3 receptor signaling | | | | | 18(15)/49 | |
| Development_Alpha-2 adrenergic receptor | | | | | 19(15)/62 | |
| activation of ERK | | | | | | |
| Development_Angiotensin signaling via beta- | | | | | 16(14)/25 | 14(12)/25 |
| Arrestin | | | | | | |
| Development_Angiotensin signaling via PYK2 | | | | | 17(15)/43 | |
| Development_Beta-adrenergic receptors regulation | | | | 16(15)/47 | 20(16)/47 | 20(17)/47 |
| of ERK | | | | | | |
| Development_Beta-adrenergic receptors | | | | 12(11)/37 | | 16(13)/37 |
| transactivation of EGFR | | | | | | |
| Development_Cross-talk between VEGF and | | | | 10(7)/26 | | |
| Angiopoietin 1 signaling pathways | | | | | | |
| Development_Delta- and kappa-type opioid | | | | | 14(13)/23 | |
| receptors signaling via beta-arrestin | | | | | | |
| Development_Dopamine D2 receptor | | | | 10(8)/24 | 11(8)/24 | |
| transactivation of EGFR | | | | | | |
| Development_EGFR signaling pathway | | | | 20(16)/63 | | |
| Development_EPO-induced MAPK pathway | | | | 14(14)/45 | 22(17)/45 | |
| Development_EPO-induced PI3K/AKT pathway | | | | 14(11)/43 | 15(9)/43 | |
| and Ca(2+) influx | | | | | | |
| Development_ERBB-family signaling | | | | 17(11)/39 | 13(7)/39 | 16(9)/39 |
| Development_FGF2-dependent induction of EMT | | | | 9(6)/20 | | |
| Development_FGFR signaling pathway | | | | | 15(9)/53 | 18(12)/53 |
| Development_Flt3 signaling | | | | 13(12)/44 | 13(12)/44 | 16(13)/44 |
| Development_Gastrin in cell growth and | | | 11(9)/62 | 16(14)/62 | 15(12)/62 | 17(14)/62 |
| proliferation | | | | | | |
| Development_GDNF family signaling | | | | 15(14)/46 | 17(13)/46 | |
| Development_GM-CSF signaling | | | | 16(15)/50 | | 16(13)/50 |
| Development_G-Proteins mediated regulation | | | | | 19(16)/46 | |
| MARK-ERK signaling | | | | | | |
| Development_Growth hormone signaling via | | | | | 13(8)/35 | |
| STATs and PLC/IP3 | | | | | | |
| Development_HGF signaling pathway | | | | 12(9)/47 | 15(10)/47 | 17(10)/47 |
| Development_HGF-dependent inhibition of TGF- | | | | | 12(11)/32 | |
| beta-induced EMT | | | | | | |
| Development_Ligand-independent activation of | | | | | 11(6)/44 | |
| ESR1 and ESR2 | | | | | | |
| Development_Melanocyte development and | | | | | | 18(10)/49 |
| pigmentation | | | | | | |
| Development_Mu-type opioid receptor signaling | | | | | 11(10)/24 | |
| via Beta-arrestin | | | | | | |
| Development_NOTCH1-mediated pathway for | | | | | 13(10)/34 | |
| NF-KB activity modulation | | | | | | |
| Development_NOTCH-induced EMT | | | | | 7(6)/19 | 8(6)/19 |
| Development_PDGF signaling via MAPK | | | 11(10)/47 | | 12(10)/47 | |
| cascades | | | | | | |
| Development_PDGF signaling via STATs and NF- | | | | 13(12)/32 | 11(10)/32 | |
| kB | | | | | | |
| Development_PEDF signaling | | | | | | 19(9)/49 |
| Development_PIP3 signaling in cardiac myocytes | | | | 17(14)/47 | 20(17)/47 | 26(19)/47 |
| Development_Prolactin receptor signaling | | | | 15(14)/58 | 18(13)/58 | |
| Development_Regulation of epithelial-to- | | | 18(17)/64 | 22(17)/64 | 26(17)/64 | 26(16)/64 |
| mesenchymal transition (EMT) | | | | | | |
| Development_Role of IL-8 in angiogenesis | 6(5)/58 | 10(7)/58 | 14(12)/58 | 31(31)/58 | 22(13)/58 | 30(13)/58 |
| Development_S1P1 receptor signaling pathway | | | | | | 19(15)/44 |
| Development_S1P1 receptor signaling via beta- | | | | 11(10)/33 | 12(11)/33 | 16(13)/33 |
| arrestin | | | | | | |
| Development_S1P3 receptor signaling pathway | | | | 18(17)/43 | 17(14)/43 | 23(20)/43 |
| Development_Slit-Robo signaling | | | | 13(12)/30 | | |
| Development_TGF-beta-dependent induction of | | | 9(9)/47 | 11(11)/47 | 12(11)/47 | 14(12)/47 |
| EMT via MAPK | | | | | | |
| Development_TGF-beta-dependent induction of | | | 11(10)/46 | 18(16)/46 | 18(14)/46 | 20(15)/46 |
| EMT via RhoA, PI3K and ILK | | | | | | |
| Development_TGF-beta-dependent induction of | | | 10(10)/35 | 11(11)/35 | 13(12)/35 | 16(12)/35 |
| EMT via SMADs | | | | | | |
| Development_Transcription regulation of | | | 12(9)/32 | 16(16)/32 | 20(16)/32 | 18(16)/32 |
| granulocyte development | | | | | | |
| Development_VEGF signaling and activation | | | | 17(14)/43 | 17(12)/43 | 19(12)/43 |
| Development_VEGF-family signaling | | | | | 14(8)/41 | 18(10)/41 |
| Development_WNT signaling pathway. Part 2 | | | | | 18(9)/53 | |
| Normal and pathological TGF-beta-mediated | | | 8(8)/33 | 11(11)/33 | 10(10)/33 | 11(11)/33 |
| regulation of cell proliferation | | | | | | |
| PGE2 pathways in cancer | | | | 19(15)/55 | 24(17)/55 | 27(20)/55 |
| Signal transduction_AKT signaling | | | | 14(10)/43 | | 19(11)/43 |
| Signal transduction_cAMP signaling | | | | | 18(15)/38 | |
| Signal transduction_Erk Interactions: Inhibition of | | | | 13(12)/34 | 14(14)/34 | |
| Erk | | | | | | |
| Signal transduction_IP3 signaling | | | | 20(14)/49 | | |
| Signal transduction_PTEN pathway | | | | | 15(12)/46 | |
| Some pathways of EMT in cancer cells | | | 9(7)/51 | 13(11)/51 | 13(10)/51 | 17(12)/51 |
| Total number of modified developmental | 1 (1) | 1 (1) | 10 (10) | 33 (28) | 43 (29) | 28 (15) |
| signaling pathways | | | | | | |
| G-protein signaling | | | | | | |
| G-protein signaling_EDG5 signaling | | | | | 14(11)/35 | |
| G-protein signaling_G-Protein alpha-12 signaling | | | 11(10)/37 | 14(14)/37 | | |
| pathway | | | | | | |
| G-protein signaling_G-Protein alpha-q signaling | | | | 15(13)/34 | 16(13)/34 | |
| cascades | | | | | | |
| G-protein signaling_G-Protein beta/gamma | | | | 12(11)/34 | 16(13)/34 | |
| signaling cascades | | | | | | |
| G-protein signaling_H-RAS regulation pathway | | 13(9)/37 | | | | 13(9)/37 |
| G-protein signaling_Proinsulin C-peptide signaling | | | 9(8)/52 | 18(16)/52 | 19(16)/52 | 20(16)/52 |
| G-protein signaling_RAC1 in cellular process | | 8(6)/36 | 12(11)/36 | 12(12)/36 | | |
| G-protein signaling_Rac2 regulation pathway | | | 9(8)/36 | 11(11)/36 | 11(10)/36 | 13(10)/36 |
| G-protein signaling_Rac3 regulation pathway | | | | | 7(4)/16 | |
| G-protein signaling_Ras family GTPases in kinase | | | 7(6)/26 | | | |
| cascades (scheme) | | | | | | |
| G-protein signaling_Regulation of cAMP levels by | | | | 12(12)/45 | 15(12)/45 | |
| ACM | | | | | | |
| G-protein signaling_Regulation of CDC42 activity | | | 8(8)/33 | | 11(8)/33 | |
| G-protein signaling_Regulation of p38 and JNK | | | 11(11)/39 | 16(16)/39 | 19(18)/39 | 19(19)/39 |
| signaling mediated by G-proteins | | | | | | |
| G-protein signaling_Regulation of RAC1 activity | | | 8(8)/36 | 13(11)/36 | 12(10)/36 | 14(11)/36 |
| G-protein signaling_RhoA regulation pathway | | | 13(10)/34 | 13(10)/34 | 15(10)/34 | 14(9)/34 |
| G-protein signaling_R-RAS regulation pathway | | 10(7)/25 | | | | 10(7)/25 |
| Membrane-bound ESR1: interaction with G- | | | | | 19(14)/51 | |
| proteins signaling | | | | | | |
| Protein folding_Membrane trafficking and signal | | | | 7(7)/19 | | 10(10)/19 |
| transduction of G-alpha heterotrimeric G-protein | | | | | | |
| Total number of modified G-protein signaling | 0 | 3 (1) | 9 (9) | 10 (10) | 12 (8) | 8 (5) |
| pathways | | | | | | |
| Chemotaxis | | | | | | |
| Chemotaxis_CCR4-induced leukocyte adhesion | | | | 14(14)/30 | 18(15)/30 | 18(18)/30 |
| Chemotaxis_CXCR4 signaling pathway | | | | | 14(12)/34 | |
| Chemotaxis_Inhibitory action of lipoxins on IL-8- | | | | 26(25)/51 | 29(27)/51 | 36(33)/51 |
| and Leukotriene B4-induced neutrophil migration | | | | | | |
| Chemotaxis_Leukocyte chemotaxis | | | 21(20)/75 | 30(28)/75 | 37(28)/75 | 37(30)/75 |
| Chemotaxis_Lipoxin inhibitory action on fMLP- | | | | 25(21)/46 | 24(20)/46 | 28(23)/46 |
| induced neutrophil chemotaxis | | | | | | |
| Total number of modified chemotaxis-related | 0 | 0 | 1 (1) | 4 (4) | 5 (5) | 4 (4) |
| pathways | | | | | | |
| Apoptosis | | | | | | |
| Apoptosis and survival_Anti-apoptotic TNFs/NF- | | | | 10(9)/41 | | |
| kB/Bcl-2 pathway | | | | | | |
| Apoptosis and survival_BAD phosphorylation | | | | 15(12)/42 | 18(14)/42 | 23(16)/42 |
| Apoptosis and survival_Beta-2 adrenergic receptor | | | | | 12(9)/23 | 18(12)/23 |
| anti-apoptotic action | | | | | | |
| Apoptosis and survival_Caspase cascade | | | | | | 19(13)/33 |
| Apoptosis and | | | | 10(6)/34 | | 11(7)/34 |
| survival_Cytoplasmic/mitochondrial transport of | | | | | | |
| proapoptotic proteins Bid, Bmf and Bim | | | | | | |
| Apoptosis and survival_DNA-damage-induced | | | | | 8(7)/15 | |
| apoptosis | | | | | | |
| Apoptosis and survival_Endoplasmic reticulum | | 14(7)/53 | | | | 19(10)/53 |
| stress response pathway | | | | | | |
| Apoptosis and survival_FAS signaling cascades | | | | | | 17(11)/43 |
| Apoptosis and survival_HTR1A signaling | | | | 21(17)/50 | 21(16)50 | |
| Apoptosis and survival_Lymphotoxin-beta | | | | 10(7)/41 | | |
| receptor signaling | | | | | | |
| Apoptosis and survival_p53-dependent apoptosis | | | | | 13(11)/29 | |
| Apoptosis and survival_Regulation of Apoptosis | | | | 12(9)/31 | | 14(11)/31 |
| by Mitochondrial Proteins | | | | | | |
| Apoptosis and survival_Role of CDK5 in neuronal | | | | 11(8)/34 | | |
| death and survival | | | | | | |
| Apoptosis and survival_Role of IAP-proteins in | | | | | | 13(7)/31 |
| apoptosis | | | | | | |
| Total number of modified apoptosis pathways | 0 | 1 (0) | 0 | 7 (4) | 5 (5) | 8 (1) |
| DNA damage | | | | | | |
| DNA damage_ATM/ATR regulation of G1/S | | 10(9)/32 | 8(8)/32 | 12(12)/32 | 18(16)/32 | 12(11)/32 |
| checkpoint | | | | | | |
| DNA damage_ATM/ATR regulation of G2/M | | | 7(7)/26 | | 10(9)/26 | 10(8)/26 |
| checkpoint | | | | | | |
| DNA damage_Brca1 as a transcription regulator | | | 9(7)/30 | 10(9)/30 | 18(14)/30 | 13(10)/30 |
| DNA damage_inhibition of telomerase activity and | | | | | 10(8)/20 | |
| cellular senescence | | | | | | |
| Total number of modified DNA damage | 0 | 1 (1) | 3 (3) | 2 (2) | 4 (4) | 3 (3) |
| pathways | | | | | | |
| Oxidative stress | | | | | | |
| Oxidative stress_Angiotensin II-induced | | | | | | 10(7)/35 |
| production of ROS | | | | | | |
| Total number of modified oxidative stress | 0 | 0 | 0 | 0 | 0 | 1 (0) |
| pathways | | | | | | |
| Transcription | | | | | | |
| Transcription_Androgen Receptor nuclear | | | 11(8)/45 | 12(7)/45 | 18(11)/45 | 15(8)/45 |
| signaling | | | | | | |
| Transcription_Assembly of RNA Polymerase II | | | 7(6)/18 | 9(8)/18 | | 18(9)/18 |
| preinitiation complex on TATA-less promoters | | | | | | |
| Transcription_CREB pathway | | | | | 16(13)/44 | 20(14)/44 |
| Transcription_Ligand-dependent activation of the | | | | | 16(8)/30 | |
| ESR1/SP pathway | | | | | | |
| Transcription_NF-kB signaling pathway | | | 8(6)/39 | 13(10)/39 | 15(10)/39 | |
| Transcription_P53 signaling pathway | | | | | 18(11)/39 | |
| Transcription_Receptor-mediated HIF regulation | | | | | | 15(9)/39 |
| Transcription_Role of AP-1 in regulation of | | | 8(8)/38 | | 11(9)/38 | |
| cellular metabolism | | | | | | |
| Transcription_Role of VDR in regulation of genes | | | | | 17(11)/59 | |
| involved in osteoporosis | | | | | | |
| Transcription_Transcription regulation of | | | 7(6)/25 | 11(9)/25 | | |
| aminoacid metabolism | | | | | | |
| Total number of modified transcription-related | 0 | 0 | 5 (4) | 4 (3) | 7 (2) | 4 (0) |
| pathways | | | | | | |
| Others | | | | | | |
| Blood coagulation_Blood coagulation | | | | | | 20(4)/39 |
| Blood coagulation_GPCRs in platelet aggregation | | | | | 28(22)/71 | 33(27)/71 |
| Blood coagulation_GPIb-IX-V-dependent platelet | | | | | | 23(20)/75 |
| activation | | | | | | |
| ENaC regulation in airways (normal and CF) | | | | | 18(11)/52 | |
| Hypoxia-induced EMT in cancer and fibrosis | | | | | 5(4)/9 | 6(5)/9 |
| Neurophysiological process_Circadian rhythm | | | 12(8)/47 | | | |
| Neurophysiological process_Receptor-mediated | | | 18(15)/45 | 23(19)/45 | 25(17)/45 | 24(17)/45 |
| axon growth repulsion | | | | | | |
| Proteolysis_Role of Parkin in the Ubiquitin- | | | | | 15(6)/24 | 14(5)/24 |
| Proteasomal Pathway | | | | | | |
| Regulation of CFTR activity (norm and CF) | | | | | 23(14)/58 | |
| Reproduction_Progesterone-mediated oocyte | | | 12(11)/40 | | | |
| maturation | | | | | | |
| Role of alpha-6/beta-4 integrins in carcinoma | | | | 15(11)/45 | | 22(15)/45 |
| progression | | | | | | |
| Translation_Regulation of EIF2 activity | | | | | 14(10)/39 | |
| Translation_Non-genomic (rapid) action of | | | | | 15(8)/40 | |
| Androgen Receptor | | | | | | |
| Transport_Clathrin-coated vesicle cycle | | | | | | 32(19)/71 |
| Transport_Macropinocytosis regulation by growth | | | 14(12)/63 | 21(18)/63 | 23(20)/63 | 25(20)/63 |
| factors | | | | | | |
| wtCFTR and delta508 traffic/Clathrin coated | | | | | 12(9)/19 | 10(9)/19 |
| vesicles formation (norm and CF) | | | | | | |
| Total number of miscellaneous pathways | 0 | 0 | 4 (3) | 3 (2) | 10 (4) | 10 (5) |

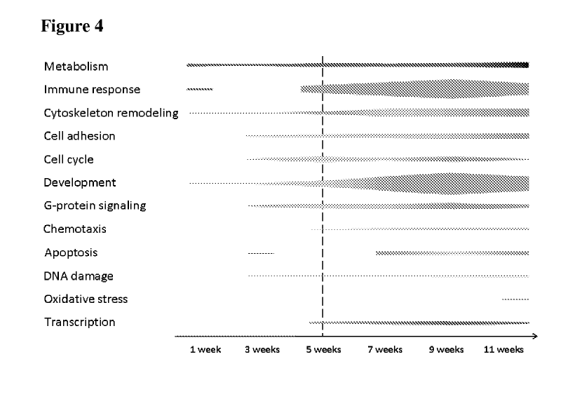

Significantly altered pathways were identified as early as one week of age, and the number of significantly altered pathways increased with age (Table 1). Early on in the disease process, a limited number of pathways showed significant alterations (FDR ≦0.05), with nine and ten pathways identified at one- and three-week-old ages, respectively. In comparison, from five to eleven weeks, the number of pathways showing significantly altered expression ranged from 71 to 157 (Tables 1 and 3, and FIG. 4).

Pathological processes that occur early in a disease are likely to be closely related to the primary defect and are targets for therapeutic intervention. The results identified nine pathways with significant alterations of gene expression at one week of age. Five of the nine pathways were involved in metabolic processes, including three in cholesterol synthesis and regulation of lipid metabolism. Consistent with known dysregulation of cholesterol synthesis in NPC cells (Beltroy et al., Hepatology 42:886-93 (2005); Ramirez et al., Pediatr. Res. 68:309-15 (2010); Liu et al., J. Lipid Res. 51:933-44 (2010); and Liu et al., Proc. Natl. Acad. Sci. USA 106:2377-82 (2009)), among the 88 genes involved in cholesterol biosynthesis, 16/17 DEG were upregulated by one week of age. Significant alterations, again consistent with prior data, were found in regulation of lipid metabolism by liver X receptor (LXR), nuclear transcription factor Y (NF-Y) and sterol regulatory element-binding protein (SREBP) (Liu et al., Proc. Natl. Acad. Sci. USA 106:2377-82 (2009); and Repa et al., J. Neurosci. 27: 14470-80 (2007)), and by peroxisome proliferator-activated receptor (PPAR) (De Windt et al., DNA Cell Biol. 26:665-71 (2007)). Other metabolic pathways with altered expression at one week of age include butanoate metabolism and linoleic acid metabolism. The latter is of particular interest since it was identified due to its downregulation of cytochrome P450 genes that encode enzymes involved in the metabolism of xenobiotics and drugs in addition to endogenous chemicals.

In addition to the metabolic disturbances, two altered pathways were involved in immune response and inflammation in the one week old data: the alternative complement and the interleukin-1 (IL-1) signaling pathways. However dysregulation of inflammatory gene sets is generally a late occurrence (FIG. 4): between 11 and 37 pathways were modified from five weeks onward, with primarily upregulated genes ( 462/677 DEG, 68%). Tnfa (Tumor necrosis factor alpha), which was previously reported as upregulated in NPC, was not significantly modified in these results (Beltroy et al., J. Lipid Res. 48:869-81 (2007); and Wu et al., Mol. Genet. Metab. 84:9-17 (2005)). However, the genes encoding its receptors Tnfrsf1a (Tnf receptor superfamily member 1 alpha) and Tnfsr1b (Tnf receptor superfamily member 1 beta) were upregulated at some time points. Five additional pathways involved in chemotaxis were also upregulated from five weeks of age onward, which likely reflect the ongoing inflammation observed in the tissue at these ages.

Only one pathway, describing the role of the IL-8 chemokine in angiogenesis, was identified as significantly altered at all six ages. It involved genes linked to regulation of metabolism, such as Srebp1 and -2, and signaling pathways regulating cell proliferation and cytoskeleton remodeling, via G-protein coupled receptors for epidermal growth factor (Egfr) and vascular endothelial growth factor (Vegfr). The gene encoding IL-8 itself did not have modified expression, and the gene encoding its receptor was only modified at 11 weeks of age. Rather than reflecting a change in angiogenesis in NPC, the modification of this pathway at all ages probably reflected both the early modification in lipid metabolism and the secondary and long term modification in intracellular signaling and cytoskeleton remodeling.

Starting at three weeks, pathway analysis suggested initial disturbances of genes involved in an increasing number of pathways in cytoskeletal and extracellular matrix remodeling, as well as disturbances in cell cycle-regulating genes, including upregulation of cyclins and cyclin-dependent kinases. In addition to the alteration of 15 pathways related to cell cycle regulation, two pathways involved in DNA damage checkpoints during cell cycle were modified from the three week time point onward (Table 3).

The second largest category of altered pathways was developmental signaling, with modifications occurring from five weeks of age. These included intracellular components of mitogen-activated protein kinase (MAPK) signaling, as well as non-canonical transforming growth factor beta (TGFβ) signaling pathways linked to epithelial-to-mesenchymal transition (EMT), with upregulation of the genes encoding the ligand Tgfb and its receptors Tgfbr1 and Tgfbr2. The data also indicated that there was an early disturbance of small GTPase function in NPC. By three weeks of age, disturbances were present in the Ras family small GTP binding protein H-Ras (Hras), Ras-related C3 botulinum toxin substrate 1 (Rac1) and the related Ras viral oncogene homolog (Rras) pathway. This dysregulation of G-protein signaling pathways was progressive, with eleven additional pathways identified, including alterations of Ras homolog gene family, member A (RhoA) and Cell division cycle 42 (Cdc42).

Although a disturbance in endoplasmic reticulum stress response was first observed at 3 weeks of age, altered pathways of apoptosis and oxidative stress, two processes that have been implicated in the NPC pathophysiological cascade (Beltroy et al., J. Lipid Res. 48:869-81 (2007); Reddy et al., PLoS One 1:e19 (2006); Klein et al., Neurobiol. Dis. 41:209-18 (2011); Fu et al., Mol. Genet. Metab. 101:214-8 (2010); and Rimkunas et al., J. Lipid Res. 50:327-33 (2009)) were relatively later processes (from seven-week-old and eleven-week-old time points onward, respectively).

### Example 3

### Genes with Consistent Differential Expression

A subset of 150 genes was identified that demonstrated altered expression at all six ages (Table 4).

TABLE 4
List of the 150 differentially expressed genes in 1-, 3-, 5-, 7-, 9-, and 11-week-old
Npc1^{-/-} mice compared to their control littermates. p-values and fold-changes (FC) are
indicated at each age.

| Gene | | 1 week | | 3 weeks | | 5 weeks | | 7 weeks | | 9 weeks | | 11 weeks | |
|---|---|---|---|---|---|---|---|---|---|---|---|---|---|
| Symbol | Gene description | p-value | FC | p-value | FC | p-value | FC | p-value | FC | p-value | FC | p-value | FC |

Cholesterol and lipid homeostasis

| | | | | | | | | | | | | | |
|---|---|---|---|---|---|---|---|---|---|---|---|---|---|
| Npc1 | Niemann-Pick disease type | 3.018E−08 | −2.164 | 2.986E−09 | −2.325 | 4.785E−10 | −2.461 | 2.765E−09 | −2.331 | 1.929E−13 | −3.137 | 1.238E−12 | −2.959 |
| | C1 | | | | | | | | | | | | |
| Npc2 | Niemann-Pick disease type | 2.042E−05 | 1.459 | 1.144E−03 | 1.322 | 1.021E−06 | 1.560 | 3.841E−09 | 1.756 | 1.344E−06 | 1.551 | 1.670E−09 | 1.786 |
| | C2 | | | | | | | | | | | | |
| Plin3 | Perilipin 3, or mannose-6-phosphate | 9.253E−08 | 1.638 | 7.720E−08 | 1.644 | 2.061E−13 | 2.146 | 8.146E−09 | 1.723 | 1.962E−14 | 2.257 | 1.035E−14 | 2.288 |
| | receptor (MPR) binding protein 1, | | | | | | | | | | | | |
| | or MPR tail-interacting protein 47 kDa | | | | | | | | | | | | |
| Apoa4 | Apolipoprotein A-IV | 3.138E−09 | 2.424 | 6.638E−12 | 2.961 | 2.507E−05 | 1.791 | 8.826E−04 | 1.563 | 5.528E−05 | 1.740 | 2.526E−07 | 2.099 |
| Es31 | Esterase 31 carboxylesterase 3 | 3.294E−09 | −4.229 | 3.722E−09 | −4.202 | 4.764E−04 | −2.158 | 1.399E−04 | −2.332 | 3.862E−07 | −3.275 | 2.865E−25 | −38.295 |
| Hexa | Hexosaminidase A (alpha subunit) | 2.827E−13 | 2.047 | 2.085E−17 | 2.493 | 2.522E−17 | 2.482 | 6.234E−24 | 3.545 | 6.115E−23 | 3.346 | 1.875E−25 | 3.887 |
| Hexb | Hexosaminidase B (beta subunit) | 4.431E−08 | 1.912 | 5.635E−10 | 2.146 | 1.501E−10 | 2.222 | 3.014E−11 | 2.318 | 3.006E−14 | 2.788 | 2.209E−14 | 2.812 |
| Hao2 | Hydroxyacid oxidase 2 | 2.687E−05 | −1.700 | 6.527E−10 | −2.355 | 1.173E−02 | −1.354 | 1.618E−11 | −2.629 | 1.292E−17 | −4.065 | 5.368E−29 | −11.18 |
| Saa4 | Serum amyloid A4 | 4.990E−02 | −1.318 | 1.533E−10 | −2.894 | 1.119E−05 | −1.936 | 8.124E−07 | −2.135 | 9.133E−09 | −2.507 | 3.095E−18 | −5.541 |
| Pltp | Phospholipid transfer protein | 2.312E−03 | −1.348 | 6.796E−03 | 1.301 | 6.441E−10 | 1.993 | 2.144E−10 | 2.046 | 3.136E−14 | 2.532 | 2.744E−17 | 3.029 |
| Hsd17b2 | 17-beta-hydroxysteroid | 5.218E−04 | −1.393 | 1.923E−08 | −1.797 | 3.156E−05 | −1.503 | 7.024E−11 | −2.045 | 5.893E−09 | −1.847 | 7.376E−13 | −2.272 |
| | dehydrogenase type 2 | | | | | | | | | | | | |
| Hmgcs1 | 3-hydroxy-3-methylglutaryl- | 7.853E−03 | 1.577 | 9.091E−04 | 1.783 | 2.933E−06 | 2.351 | 2.119E−02 | 1.480 | 2.452E−02 | 1.466 | 2.918E−02 | −1.448 |
| | CoA synthase 1 | | | | | | | | | | | | |
| Elovl7 | Elongation of long chain | 3.127E−06 | 1.781 | 2.403E−12 | 2.677 | 3.011E−11 | 2.489 | 1.782E−17 | 3.815 | 1.719E−17 | 3.820 | 8.814E−24 | 6.270 |
| | fatty acids and sphingolipids | | | | | | | | | | | | |
| | family member 7 | | | | | | | | | | | | |

Transription factors and developmental signaling pathways

| | | | | | | | | | | | | | |
|---|---|---|---|---|---|---|---|---|---|---|---|---|---|
| Arhgef2 | Rho/Rac guanine nucleotide | 3.432E−06 | 1.313 | 3.544E−09 | 1.445 | 1.268E−12 | 1.609 | 3.364E−23 | 2.333 | 1.285E−24 | 2.472 | 2.953E−31 | 3.368 |
| | exchange factor 2 | | | | | | | | | | | | |
| Dusp3 | Dual specificity phosphatase 3 | 4.089E−05 | 1.417 | 7.278E−06 | 1.472 | 1.647E−05 | 1.446 | 1.232E−12 | 2.021 | 9.255E−04 | 1.315 | 1.462E−10 | 1.836 |
| Fpr1 | Formyl peptide receptor 1 | 2.759E−03 | −1.399 | 3.397E−04 | −1.505 | 4.226E−04 | −1.494 | 1.675E−04 | −1.540 | 7.748E−07 | −1.810 | 1.387E−06 | −1.780 |
| Gpr137b | G-protein coupled receptor | 2.133E−03 | 1.691 | 6.003E−09 | 3.030 | 1.172E−14 | 5.270 | 3.104E−22 | 12.013 | 4.293E−24 | 15.100 | 6.455E−25 | 16.777 |
| | 137B | | | | | | | | | | | | |
| Inhbc | Inhibin beta C | 2.072E−07 | −1.835 | 2.633E−03 | −1.384 | 8.374E−03 | −1.326 | 3.091E−06 | −1.703 | 3.279E−08 | −1.928 | 2.061E−18 | −3.661 |
| Jun | c-Jun transcription factor | 1.696E−02 | 1.684 | 3.159E−02 | 1.595 | 2.325E−02 | 1.639 | 3.078E−07 | 3.394 | 5.846E−04 | 2.161 | 7.753E−05 | 2.460 |
| Map3k1 | Mitogen-activated protein | 9.703E−03 | 1.317 | 2.256E−06 | 1.715 | 5.931E−06 | 1.669 | 1.107E−05 | 1.640 | 5.993E−06 | 1.669 | 9.524E−09 | 1.987 |
| | kinase kinase kinase 1 | | | | | | | | | | | | |
| Mfge8 | Milk fat globule-EGF factor | 9.668E−05 | 1.369 | 4.795E−06 | 1.459 | 4.642E−05 | 1.391 | 1.333E−14 | 2.141 | 2.068E−19 | 2.700 | 1.640E−27 | 4.294 |
| | 8 protein | | | | | | | | | | | | |
| Mvp | Major vault protein | 7.797E−06 | 1.457 | 4.774E−05 | 1.401 | 1.157E−05 | 1.445 | 3.362E−12 | 1.951 | 5.739E−09 | 1.686 | 1.320E−11 | 1.899 |
| Pak1 | p21/CDC42/Rac-activated | 6.209E−04 | 1.455 | 7.416E−05 | 1.556 | 2.610E−09 | 2.067 | 4.599E−12 | 2.445 | 7.920E−11 | 2.267 | 7.873E−13 | 2.563 |
| | kinase 1 | | | | | | | | | | | | |
| Prok1 | Prokineticin 1 | 4.233E−03 | −1.558 | 2.979E−03 | −1.587 | 5.065E−04 | −1.730 | 5.406E−04 | −1.725 | 5.835E−05 | −1.906 | 1.025E−06 | −2.251 |
| Rhoq | Ras homolog gene family, | 9.938E−06 | 1.592 | 7.808E−10 | 2.019 | 8.287E−07 | 1.698 | 2.685E−11 | 2.193 | 1.872E−06 | 1.663 | 2.266E−12 | 2.331 |
| | member Q | | | | | | | | | | | | |
| Rragd | Ras-related GTP binding D | 3.610E−12 | 2.299 | 2.754E−16 | 2.925 | 6.738E−14 | 2.539 | 7.123E−19 | 3.436 | 4.897E−18 | 3.258 | 7.316E−26 | 5.636 |
| Spic | Spi-C transcription factor | 3.785E−05 | 1.577 | 1.802E−09 | 2.065 | 2.170E−04 | 1.496 | 9.719E−03 | 1.314 | 3.341E−03 | 1.367 | 6.931E−12 | 2.387 |

Cell cycle

| | | | | | | | | | | | | | |
|---|---|---|---|---|---|---|---|---|---|---|---|---|---|
| Hist1h1b | Histone gene cluster 1, H1 | 1.160E−04 | 1.708 | 2.055E−06 | 1.978 | 3.569E−05 | 1.786 | 1.474E−02 | 1.385 | 1.378E−03 | 1.546 | 1.916E−02 | 1.367 |
| | histone family, member B | | | | | | | | | | | | |
| Lgals1 | Lectin galactoside-binding | 2.554E−07 | 1.708 | 2.199E−09 | 1.912 | 9.611E−13 | 2.293 | 4.248E−21 | 3.761 | 6.114E−22 | 3.977 | 3.122E−22 | 4.055 |
| | soluble 1 | | | | | | | | | | | | |

Cell adhesion and cytoskeleton

| | | | | | | | | | | | | | |
|---|---|---|---|---|---|---|---|---|---|---|---|---|---|
| Cdh1 | E-cadherin | 7.441E−04 | 1.397 | 2.349E−13 | 2.413 | 4.108E−12 | 2.252 | 1.101E−08 | 1.864 | 5.817E−09 | 1.892 | 3.658E−15 | 2.676 |
| Krt8 | Keratin 8 | 2.614E−03 | 1.387 | 1.676E−07 | 1.852 | 9.233E−08 | 1.882 | 3.423E−11 | 2.322 | 1.022E−07 | 1.877 | 8.409E−09 | 2.007 |
| Tuba8 | Tubulin alpha 8 | 7.415E−06 | 1.634 | 3.918E−08 | 1.877 | 1.566E−05 | 1.600 | 1.071E−06 | 1.721 | 2.492E−10 | 2.136 | 2.380E−18 | 3.490 |
| Vcam1 | Vascular cell adhesion | 1.030E−02 | 1.388 | 2.520E−10 | 2.554 | 9.106E−10 | 2.453 | 4.764E−07 | 2.010 | 2.156E−04 | 1.627 | 1.635E−09 | 2.408 |
| | molecule 1 | | | | | | | | | | | | |

Marker of mature liver

| | | | | | | | | | | | | | |
|---|---|---|---|---|---|---|---|---|---|---|---|---|---|
| Aldoa | Aldolase A, or fructose- | 4.286E−04 | 1.344 | 8.641E−06 | 1.471 | 4.281E−04 | 1.344 | 3.508E−12 | 1.989 | 4.248E−10 | 1.806 | 4.379E−16 | 2.401 |
| | bisphosphate aldolase | | | | | | | | | | | | |

Arachidonic acid and drug metabolism

| | | | | | | | | | | | | | |
|---|---|---|---|---|---|---|---|---|---|---|---|---|---|
| Anxa5 | Annexin A5 | 5.362E−07 | 1.612 | 3.411E−12 | 2.092 | 1.421E−15 | 2.488 | 5.903E−19 | 2.994 | 4.264E−21 | 3.394 | 6.367E−20 | 3.166 |
| Cyp2c37 | Cytochrome P450, family 2, | 1.213E−06 | −1.716 | 1.259E−09 | −2.050 | 4.038E−03 | −1.349 | 1.895E−08 | −1.913 | 3.491E−03 | −1.356 | 3.733E−06 | −1.665 |
| | subfamily c, polypeptide 37 | | | | | | | | | | | | |
| Cyp2c40 | Cytochrome P450, family 2, | 3.147E−07 | −2.032 | 1.445E−08 | −2.242 | 5.466E−06 | −1.850 | 3.907E−16 | −3.914 | 4.093E−14 | −3.357 | 3.478E−18 | −4.603 |
| | subfamily c, polypeptide 40 | | | | | | | | | | | | |
| Cyp2c50 | Cytochrome P450, family 2, | 1.596E−15 | −3.145 | 7.034E−16 | −3.221 | 1.112E−03 | −1.444 | 3.455E−08 | −1.970 | 1.037E−02 | −1.328 | 1.456E−12 | −2.596 |
| | subfamily c, polypeptide 50 | | | | | | | | | | | | |
| Cyp2c54 | Cytochrome P450, family 2, | 1.031E−07 | −2.527 | 1.259E−11 | −3.597 | 2.983E−06 | −2.205 | 1.551E−09 | −2.981 | 4.259E−07 | −2.388 | 1.914E−15 | −5.116 |
| | subfamily c, polypeptide 54 | | | | | | | | | | | | |
| Cyp2c67 | Cytochrome P450, family 2, | 8.052E−09 | −2.493 | 2.815E−07 | −2.200 | 7.743E−06 | −1.949 | 1.853E−10 | −2.842 | 1.178E−06 | −2.089 | 7.807E−15 | −4.053 |
| | subfamily c, polypeptide 67 | | | | | | | | | | | | |
| Cyp2c68 | Cytochrome P450, family 2, | 1.654E−03 | −1.484 | 3.438E−05 | −1.710 | 9.911E−04 | −1.514 | 8.273E−11 | −2.566 | 7.292E−04 | −1.532 | 1.804E−09 | −2.336 |
| | subfamily c, polypeptide 68 | | | | | | | | | | | | |
| Cyp2d13 | Cytochrome P450, family 2, | 2.460E−05 | −1.722 | 9.518E−04 | −1.512 | 1.229E−03 | −1.497 | 1.436E−05 | −1.754 | 6.736E−03 | −1.396 | 1.256E−15 | −3.593 |
| | subfamily d, polypeptide 13 | | | | | | | | | | | | |
| Cyp2d40 | Cytochrome P450, family 2, | 3.061E−04 | −1.722 | 4.783E−03 | −1.515 | 1.148E−03 | −1.623 | 1.678E−08 | −2.519 | 1.337E−04 | −1.784 | 1.301E−14 | −4.208 |
| | subfamily d, polypeptide 40 | | | | | | | | | | | | |
| Cyp4f14 | Cytochrome P450, family 4, | 2.402E−04 | −1.479 | 4.128E−15 | −2.847 | 6.409E−05 | −1.539 | 1.734E−13 | −2.579 | 4.352E−13 | −2.518 | 1.646E−25 | −5.903 |
| | subfamily f, polypeptide 14 | | | | | | | | | | | | |
| Gpx4 | Glutathione peroxidase 4 | 3.451E−10 | 1.433 | 5.092E−09 | 1.387 | 6.337E−08 | 1.345 | 6.522E−10 | 1.422 | 1.039E−12 | 1.539 | 7.559E−18 | 1.793 |
| Gpx3 | Glutathione peroxidase 3 | 2.459E−02 | 1.312 | 1.101E−04 | 1.629 | 1.444E−05 | 1.745 | 1.924E−11 | 2.640 | 4.185E−08 | 2.096 | 1.774E−18 | 4.405 |
| Hpgds | Prostaglandin-D synthase | 3.344E−10 | 2.089 | 2.179E−14 | 2.665 | 4.471E−16 | 2.951 | 6.676E−21 | 4.040 | 1.776E−27 | 6.669 | 4.736E−30 | 8.363 |
| Ugt2b1 | UDP glucuronosyltransferase | 3.356E−05 | −1.462 | 8.137E−06 | −1.512 | 2.826E−03 | −1.302 | 8.809E−10 | −1.851 | 1.549E−06 | −1.571 | 6.338E−14 | −2.278 |
| | 2 family polypeptide B1 | | | | | | | | | | | | |

Immune response and inflammation

| | | | | | | | | | | | | | |
|---|---|---|---|---|---|---|---|---|---|---|---|---|---|
| C8g | Complement component 8 | 6.982E−12 | −1.746 | 1.463E−11 | −1.724 | 2.516E−06 | −1.406 | 2.884E−12 | −1.772 | 1.415E−11 | −1.725 | 7.456E−23 | −2.791 |
| | gamma polypeptide | | | | | | | | | | | | |
| Ccr3 | Chemokine C-C motif | 2.758E−06 | −1.862 | 1.122E−03 | −1.508 | 6.267E−10 | −2.417 | 2.699E−10 | −2.480 | 1.020E−11 | −2.742 | 8.834E−08 | −2.076 |
| | receptor 3 | | | | | | | | | | | | |
| Cd68 | Macrophage antigen cd68 | 7.056E−03 | 1.430 | 1.939E−12 | 3.099 | 2.139E−17 | 4.582 | 6.316E−26 | 10.108 | 7.009E−27 | 11.189 | 1.335E−28 | 13.545 |
| Cd83 | Cd83 antigen | 1.677E−03 | 1.425 | 5.541E−09 | 2.079 | 3.402E−09 | 2.107 | 1.531E−10 | 2.293 | 2.411E−13 | 2.741 | 1.190E−16 | 3.412 |
| Cd84 | Cd84 antigen | 5.583E−03 | 1.406 | 1.445E−09 | 2.329 | 7.497E−12 | 2.730 | 2.236E−20 | 5.170 | 1.039E−24 | 7.555 | 1.475E−25 | 8.185 |
| Cd163 | Cd163 antigen or hemoglobin | 2.503E−11 | −2.416 | 9.551E−19 | −3.965 | 6.726E−23 | −5.436 | 7.180E−26 | −6.981 | 1.378E−24 | −6.249 | 2.239E−26 | −7.302 |
| | scavenger receptor | | | | | | | | | | | | |
| Clec1b | C-type lectin domain family | 8.622E−03 | 1.349 | 2.333E−04 | 1.540 | 4.276E−08 | 1.996 | 8.625E−09 | 2.089 | 5.280E−08 | 1.984 | 1.403E−10 | 2.345 |
| | 1, member B | | | | | | | | | | | | |
| Clec7a | C-type lectin domain family | 2.278E−04 | 2.024 | 3.677E−15 | 6.564 | 1.400E−19 | 10.966 | 1.532E−22 | 15.986 | 3.575E−24 | 19.957 | 2.375E−23 | 17.822 |
| | 7, member A | | | | | | | | | | | | |
| Gpnmb | Glycoprotein nmb | 5.018E−03 | 1.881 | 4.847E−17 | 12.506 | 9.733E−23 | 29.251 | 1.671E−28 | 80.723 | 6.504E−29 | 87.402 | 2.782E−30 | 114.90 |
| Il1rap | Interleukin 1 receptor | 6.664E−04 | −1.321 | 1.376E−06 | −1.516 | 5.320E−05 | −1.402 | 5.922E−09 | −1.693 | 7.408E−08 | −1.609 | 2.776E−17 | −2.500 |
| | accessory protein | | | | | | | | | | | | |
| Lgals3 | Lectin galactoside-binding | 2.328E−02 | 1.476 | 2.642E−11 | 3.913 | 1.280E−15 | 6.040 | 2.816E−22 | 12.765 | 1.167E−21 | 11.843 | 7.048E−24 | 15.605 |
| | soluble 3 or macrophage | | | | | | | | | | | | |
| | galactose specific lectin 2 | | | | | | | | | | | | |
| Ly6d | Lymphocyte antigen 6 | 1.193E−03 | 1.633 | 1.608E−07 | 2.351 | 2.902E−10 | 2.969 | 2.119E−15 | 4.621 | 1.390E−09 | 2.803 | 4.944E−08 | 2.457 |
| | complex, locus D | | | | | | | | | | | | |
| Ly9 | Lymphocyte antigen 9 | 2.074E−03 | 1.421 | 9.305E−05 | 1.579 | 2.103E−06 | 1.773 | 5.413E−13 | 2.714 | 9.129E−16 | 3.262 | 9.653E−16 | 3.257 |
| Tyrobp | TYRO protein tyrosine | 1.124E−02 | 1.312 | 6.466E−06 | 1.670 | 3.597E−08 | 1.925 | 1.351E−17 | 3.473 | 1.803E−17 | 3.444 | 1.799E−19 | 3.951 |
| | kinase binding protein | | | | | | | | | | | | |

Solute carrier family

| | | | | | | | | | | | | | |
|---|---|---|---|---|---|---|---|---|---|---|---|---|---|
| Sat1 | Solute carrier family 26 | 1.590E−05 | 1.595 | 1.517E−03 | 1.391 | 5.079E−09 | 1.969 | 2.352E−05 | 1.577 | 2.825E−09 | 1.999 | 2.069E−14 | 2.704 |
| | member 1 | | | | | | | | | | | | |
| Slc7a8 | Solute carrier family 7 | 1.189E−03 | 1.369 | 3.879E−11 | 2.105 | 6.425E−11 | 2.080 | 6.857E−19 | 3.279 | 9.943E−19 | 3.247 | 3.898E−24 | 4.644 |
| | member 8 | | | | | | | | | | | | |
| Slc13a3 | Solute carrier family 13 | 2.062E−03 | −1.460 | 6.423E−04 | 1.527 | 2.108E−02 | 1.321 | 1.550E−04 | 1.607 | 6.551E−04 | 1.526 | 1.737E−05 | 1.731 |
| | member 3 | | | | | | | | | | | | |
| Slc22a7 | Solute carrier family 22, | 1.799E−07 | −2.069 | 1.570E−06 | −1.929 | 4.366E−09 | −2.329 | 1.938E−18 | −4.701 | 8.848E−23 | −6.838 | 1.384E−29 | −13.684 |
| | member 7 | | | | | | | | | | | | |
| Slc25a4 | Solute carrier family 25, | 7.019E−03 | 1.347 | 6.761E−09 | 2.055 | 1.691E−08 | 2.004 | 4.670E−05 | 1.598 | 4.177E−09 | 2.082 | 2.205E−13 | 2.725 |
| | member 4 | | | | | | | | | | | | |
| Slc37a2 | Solute carrier family 37 | 8.523E−03 | 1.303 | 1.680E−07 | 1.779 | 2.155E−09 | 1.984 | 2.718E−19 | 3.590 | 3.455E−19 | 3.565 | 1.601E−23 | 4.820 |
| | member 2 | | | | | | | | | | | | |

Lysosomal proteins

| | | | | | | | | | | | | | |
|---|---|---|---|---|---|---|---|---|---|---|---|---|---|
| Cln6 | Ceroid lipofuscinosis | 1.075E−03 | 1.367 | 3.540E−13 | 2.321 | 1.068E−11 | 2.144 | 4.127E−16 | 2.732 | 2.470E−14 | 2.473 | 1.314E−20 | 3.580 |
| | neuronal 6 | | | | | | | | | | | | |
| Ctsa | Cathepsin A | 1.852E−05 | 1.308 | 3.217E−10 | 1.543 | 2.373E−11 | 1.603 | 1.334E−10 | 1.563 | 1.403E−12 | 1.671 | 4.079E−11 | 1.590 |
| Ctsd | Cathepsin D | 9.995E−06 | 1.371 | 4.036E−13 | 1.829 | 4.130E−17 | 2.148 | 3.496E−24 | 2.973 | 9.721E−25 | 3.059 | 4.039E−27 | 3.475 |
| Ctsz | Cathepsin Z | 8.063E−16 | 1.784 | 1.438E−16 | 1.830 | 4.252E−16 | 1.801 | 2.866E−15 | 1.752 | 1.829E−21 | 2.180 | 1.452E−22 | 2.275 |

Other genes

| | | | | | | | | | | | | | |
|---|---|---|---|---|---|---|---|---|---|---|---|---|---|
| Accn5 | Amiloride-sensitive cation | 2.400E−02 | −1.316 | 3.981E−08 | −2.109 | 1.135E−02 | −1.363 | 4.950E−06 | −1.813 | 3.263E−06 | −1.838 | 6.240E−11 | −2.564 |
| | channel 5 | | | | | | | | | | | | |
| Acp5 | Acid phosphatase 5 | 3.537E−10 | 1.522 | 9.776E−11 | 1.550 | 3.031E−09 | 1.476 | 5.943E−13 | 1.668 | 4.149E−15 | 1.795 | 9.763E−17 | 1.900 |
| Acy3 | Aspartoacylase 3 | 1.476E−06 | −1.579 | 7.035E−06 | −1.523 | 1.469E−03 | −1.330 | 3.319E−07 | −1.633 | 8.477E−07 | −1.599 | 3.425E−16 | −2.583 |
| Aldoc | Aldolase C fructose- | 3.147E−03 | 1.442 | 2.147E−09 | 2.309 | 3.811E−03 | 1.430 | 2.216E−02 | 1.322 | 2.840E−02 | 1.306 | 5.883E−03 | 1.404 |
| | bisphosphate | | | | | | | | | | | | |
| Appl2 | Chymotrypsin plasmin factor | 4.780E−05 | 1.393 | 3.806E−14 | 2.106 | 5.892E−08 | 1.597 | 1.277E−06 | 1.503 | 2.677E−09 | 1.696 | 2.172E−10 | 1.780 |
| | XIA and plasma and | | | | | | | | | | | | |
| | glandular kallikrein | | | | | | | | | | | | |
| Aqp7 | Aquaporin 7 | 8.902E−05 | 1.583 | 2.313E−09 | 2.155 | 5.306E−10 | 2.245 | 3.002E−10 | 2.281 | 1.602E−12 | 2.640 | 7.836E−12 | 2.525 |
| Atp8a1 | ATPase aminophospholipid | 1.333E−03 | 1.423 | 7.279E−07 | 1.786 | 2.288E−03 | 1.397 | 1.685E−08 | 1.980 | 8.637E−06 | 1.666 | 3.152E−04 | 1.493 |
| | transporter class I type 8A | | | | | | | | | | | | |
| | member 1 | | | | | | | | | | | | |
| Bglap2 | Bone gamma- | 3.511E−05 | 1.493 | 4.860E−05 | 1.480 | 1.175E−09 | 1.905 | 3.108E−08 | 1.767 | 2.400E−06 | 1.596 | 7.373E−05 | 1.465 |
| | carboxyglutamate protein 2 | | | | | | | | | | | | |
| Camk2d | Calcium/calmodulin- | 1.077E−04 | 1.396 | 9.029E−09 | 1.710 | 1.277E−09 | 1.780 | 1.528E−12 | 2.044 | 5.423E−11 | 1.899 | 3.122E−20 | 3.027 |
| | dependent protein kinase II | | | | | | | | | | | | |
| | delta | | | | | | | | | | | | |
| Car1 | Carbonic anhydrase I | 1.427E−02 | −1.419 | 4.380E−07 | −2.195 | 2.459E−05 | −1.886 | 4.629E−05 | −1.839 | 1.011E−07 | −2.315 | 4.967E−16 | −4.606 |
| Cd209f | Cd209f antigen | 1.723E−22 | −6.339 | 4.563E−11 | −2.618 | 1.496E−10 | −2.525 | 4.770E−08 | −2.116 | 6.290E−07 | −1.952 | 8.471E−07 | −1.934 |
| Cd63 | CD63 antigen | 1.282E−03 | 1.669 | 1.972E−13 | 4.171 | 5.511E−09 | 2.804 | 1.779E−13 | 4.188 | 5.275E−17 | 5.829 | 2.785E−18 | 6.609 |
| Ces6 | Carboxylesterase 6 | 1.287E−03 | −1.398 | 1.454E−07 | −1.804 | 1.013E−04 | −1.511 | 1.617E−13 | −2.556 | 2.453E−12 | −2.383 | 5.546E−19 | −3.597 |
| Cmah | Cytidine monophosphate-N- | 8.315E−05 | −1.460 | 3.064E−09 | −1.863 | 1.228E−08 | −1.805 | 2.468E−19 | −3.248 | 9.160E−19 | −3.138 | 4.791E−34 | −9.951 |
| | acetylneuraminic acid | | | | | | | | | | | | |
| | hydroxylase | | | | | | | | | | | | |
| Cpne8 | Copine VIII | 1.835E−03 | 1.399 | 5.570E−06 | 1.671 | 4.303E−07 | 1.794 | 1.400E−07 | 1.849 | 4.947E−05 | 1.569 | 6.160E−07 | 1.776 |
| Dpp7 | Dipeptidyl-peptidase 7 | 4.872E−05 | 1.397 | 1.706E−05 | 1.430 | 2.947E−06 | 1.483 | 1.046E−08 | 1.660 | 1.390E−07 | 1.578 | 3.386E−08 | 1.623 |
| Endod1 | Endonuclease domain | 1.598E−03 | 1.385 | 8.582E−09 | 1.933 | 6.847E−05 | 1.525 | 1.321E−08 | 1.912 | 7.948E−08 | 1.827 | 4.032E−06 | 1.649 |
| | containing 1, may act as | | | | | | | | | | | | |
| | DNase and RNase | | | | | | | | | | | | |
| Epb4.1l3 | Erythrocyte protein band 4.1- | 7.935E−07 | 1.465 | 7.593E−11 | 1.728 | 3.520E−13 | 1.901 | 2.295E−12 | 1.838 | 1.320E−16 | 2.199 | 1.291E−19 | 2.522 |
| | like 3 | | | | | | | | | | | | |
| Fnip2 | Folliculin interacting protein 2 | 9.738E−04 | 1.660 | 3.351E−07 | 2.313 | 1.005E−02 | 1.475 | 1.960E−04 | 1.786 | 2.108E−02 | 1.414 | 7.981E−04 | 1.676 |
| Folr2 | Folate receptor 2 | 1.747E−07 | −1.559 | 4.862E−10 | −1.746 | 4.862E−12 | −1.907 | 2.681E−13 | −2.017 | 8.875E−16 | −2.260 | 5.416E−15 | −2.179 |
| Fuca2 | Fucosidase alpha-L-2 | 1.378E−03 | 1.337 | 2.660E−09 | 1.832 | 1.392E−11 | 2.056 | 2.698E−06 | 1.567 | 1.347E−14 | 2.404 | 8.881E−14 | 2.303 |
| Gdpd1 | Glycerophosphodiester | 9.034E−03 | 1.346 | 1.203E−03 | 1.455 | 6.906E−05 | 1.602 | 8.369E−10 | 2.231 | 3.247E−14 | 2.975 | 1.262E−17 | 3.764 |
| | phosphodiesterase domain | | | | | | | | | | | | |
| | containing 1 | | | | | | | | | | | | |
| Gria3 | Glutamate receptor | 1.229E−03 | 1.502 | 1.082E−02 | 1.371 | 4.700E−05 | 1.692 | 5.098E−05 | 1.687 | 1.093E−04 | 1.643 | 1.063E−04 | 1.645 |
| | ionotrophic AMPA 3 | | | | | | | | | | | | |
| Gucy2c | Guanylate cyclase 2C | 1.283E−12 | 2.377 | 1.001E−14 | 2.687 | 7.303E−12 | 2.276 | 1.768E−13 | 2.498 | 7.767E−07 | 1.707 | 4.844E−06 | 1.627 |
| H28 | Histocompatibility 28 | 3.860E−02 | 1.386 | 1.603E−03 | 1.665 | 9.760E−03 | 1.509 | 1.393E−05 | 2.074 | 2.100E−07 | 2.468 | 3.520E−03 | 1.598 |
| H2-T24 | Histocompatibility 2, T | 5.638E−05 | 1.502 | 2.386E−05 | 1.537 | 1.274E−03 | 1.374 | 2.119E−12 | 2.285 | 2.737E−14 | 2.541 | 8.207E−11 | 2.093 |
| | region locus 24 | | | | | | | | | | | | |
| Impact | Impact homolog | 1.394E−03 | 1.316 | 9.234E−08 | 1.646 | 1.708E−04 | 1.389 | 8.406E−11 | 1.906 | 1.368E−09 | 1.798 | 1.481E−13 | 2.178 |
| Large | Like-glycosyltransferase | 2.071E−03 | 1.318 | 1.053E−06 | 1.594 | 2.522E−12 | 2.121 | 1.468E−19 | 3.134 | 3.762E−17 | 2.734 | 9.159E−21 | 3.366 |
| Ngp | Neutrophilic granule protein | 3.125E−03 | −1.627 | 1.160E−02 | 1.509 | 1.591E−02 | 1.480 | 1.224E−07 | 2.582 | 2.352E−06 | 2.282 | 3.342E−09 | 2.989 |
| Np1 | N-acetylneuraminate | 6.344E−06 | 1.512 | 1.462E−10 | 1.906 | 3.767E−10 | 1.868 | 1.687E−10 | 1.900 | 1.887E−12 | 2.091 | 9.681E−18 | 2.747 |
| | pyruvate lyase | | | | | | | | | | | | |
| P2rx4 | Purinergic receptor P2X | 4.415E−08 | 1.661 | 5.434E−12 | 2.001 | 1.139E−13 | 2.170 | 6.510E−16 | 2.425 | 5.111E−18 | 2.705 | 6.902E−18 | 2.686 |
| | ligand-gated ion channel 4 | | | | | | | | | | | | |
| Paox | Polyamine oxidase | 6.755E−03 | −1.340 | 5.548E−06 | −1.682 | 1.080E−02 | −1.316 | 6.153E−11 | −2.290 | 1.206E−08 | −1.990 | 4.802E−11 | −2.305 |
| Plscr1 | Phospholipid scramblase 1 | 1.634E−03 | 1.327 | 1.197E−07 | 1.675 | 3.602E−06 | 1.550 | 1.985E−08 | 1.743 | 1.004E−05 | 1.513 | 6.187E−05 | 1.447 |
| Ppm1h | Protein phosphatase | 9.418E−05 | 1.397 | 1.093E−03 | 1.315 | 2.874E−05 | 1.436 | 2.248E−10 | 1.837 | 1.151E−04 | 1.390 | 3.935E−10 | 1.816 |
| | Mg2+/Mn2+ dependent 1H | | | | | | | | | | | | |
| Renbp | Renin binding protein | 5.006E−15 | 2.936 | 3.142E−17 | 3.389 | 3.853E−19 | 3.861 | 5.678E−22 | 4.740 | 3.505E−23 | 5.201 | 3.511E−24 | 5.629 |
| Scpep1 | Serine carboxypeptidase 1 | 1.258E−04 | 1.362 | 7.857E−12 | 1.892 | 7.102E−14 | 2.074 | 5.587E−07 | 1.525 | 2.640E−11 | 1.849 | 9.411E−09 | 1.652 |
| Sdr42e1 | Short chain | 4.135E−03 | −1.350 | 6.572E−04 | −1.436 | 1.674E−05 | −1.602 | 2.562E−06 | −1.687 | 3.778E−10 | −2.123 | 8.790E−11 | −2.204 |
| | dehydrogenase/reductase | | | | | | | | | | | | |
| | family 42E member 1 | | | | | | | | | | | | |
| Serpina1c | Serine (or cysteine) peptidase | 4.811E−03 | −1.529 | 3.478E−05 | −1.914 | 7.800E−05 | −1.851 | 3.471E−09 | −2.728 | 1.388E−06 | −2.177 | 5.148E−16 | −4.933 |
| | inhibitor clade A member 1c | | | | | | | | | | | | |
| Ugt3a1 | UDP glycosyltransferase 3 | 1.122E−05 | −2.084 | 7.619E−06 | −2.118 | 2.031E−06 | −2.238 | 2.539E−13 | −4.188 | 4.346E−13 | −4.100 | 1.069E−28 | −22.816 |
| | family polypeptide 1 | | | | | | | | | | | | |

Genes with unknown function and pseudogenes

| | | | | | | | | | | | | | |
|---|---|---|---|---|---|---|---|---|---|---|---|---|---|
| 1700029I01Rik | RIKEN cDNA 1700029I01 | 3.987E−03 | 1.315 | 4.703E−06 | 1.585 | 2.843E−07 | 1.697 | 5.805E−13 | 2.308 | 4.275E−17 | 2.913 | 1.065E−15 | 2.687 |
| | gene | | | | | | | | | | | | |
| 1700112E06Rik | RIKEN cDNA 1700112E06 | 1.445E−02 | 1.330 | 4.748E−06 | 1.768 | 2.914E−04 | 1.547 | 2.267E−09 | 2.217 | 2.844E−09 | 2.203 | 1.190E−09 | 2.259 |
| | gene | | | | | | | | | | | | |
| 2010003K11Rik | RIKEN cDNA 20100003K11 | 2.195E−03 | 1.552 | 1.102E−07 | 2.289 | 7.089E−08 | 2.325 | 3.085E−09 | 2.597 | 5.507E−07 | 2.160 | 1.728E−02 | 1.400 |
| | gene | | | | | | | | | | | | |
| 2200001I15Rik | RIKEN cDNA 2200001I15 | 3.234E−04 | −1.449 | 1.958E−03 | −1.370 | 1.237E−03 | −1.390 | 1.444E−05 | −1.582 | 1.040E−04 | −1.497 | 7.390E−06 | −1.611 |
| | gene | | | | | | | | | | | | |
| 2810007J24Rik | RIKEN cDNA 28100007J24 | 2.846E−07 | −2.454 | 1.059E−05 | −2.110 | 6.039E−11 | −3.435 | 5.245E−20 | −8.282 | 6.908E−19 | −7.361 | 1.315E−30 | −31.331 |
| | gene | | | | | | | | | | | | |
| 9530008L14Rik | RIKEN cDNA 9530008L14 | 4.581E−06 | −1.358 | 1.168E−05 | −1.337 | 2.033E−08 | −1.481 | 1.608E−15 | −1.914 | 1.365E−17 | −2.073 | 1.853E−27 | −3.237 |
| | gene | | | | | | | | | | | | |
| Abhd3 | Abhydrolase domain | 1.329E−08 | −1.445 | 3.959E−10 | −1.519 | 1.064E−05 | −1.309 | 3.819E−13 | −1.678 | 3.074E−10 | −1.525 | 1.278E−21 | −2.284 |
| | containing protein 3 | | | | | | | | | | | | |
| AI317395 | Potential sodium-dependent | 1.540E−03 | −1.310 | 1.763E−06 | −1.539 | 2.978E−04 | −1.367 | 1.617E−07 | −1.621 | 7.793E−06 | −1.490 | 3.406E−10 | −1.843 |
| | glucose transporter 1A | | | | | | | | | | | | |
| Arl8a | ADP-ribosylation factor-like | 1.835E−05 | 1.397 | 1.498E−08 | 1.600 | 5.235E−10 | 1.701 | 9.178E−14 | 1.995 | 2.158E−12 | 1.881 | 7.758E−19 | 2.511 |
| | 8A | | | | | | | | | | | | |
| BC014805 | Hypothetical protein | 1.198E−07 | −2.446 | 1.250E−05 | −2.033 | 8.500E−05 | −1.874 | 5.723E−09 | −2.749 | 2.734E−12 | −3.674 | 1.965E−17 | −5.885 |
| BC025446 | cDNA sequence BC025446 | 3.660E−03 | −1.315 | 1.496E−03 | −1.352 | 5.651E−04 | −1.390 | 2.132E−07 | −1.699 | 1.219E−07 | −1.722 | 6.006E−16 | −2.696 |
| Bglap-rs1 | Bone gamma- | 1.186E−04 | 2.170 | 9.387E−06 | 2.486 | 1.586E−07 | 3.053 | 5.864E−05 | 2.256 | 5.276E−03 | 1.724 | 6.458E−03 | 1.700 |
| | carboxyglutamate protein, | | | | | | | | | | | | |
| | related sequence 1 | | | | | | | | | | | | |
| C730048C13Rik | RIKEN cDNA C730048C13 | 4.068E−10 | −2.629 | 6.000E−11 | −2.800 | 2.268E−04 | −1.662 | 1.802E−07 | −2.147 | 1.846E−08 | −2.318 | 1.240E−19 | −5.642 |
| | gene | | | | | | | | | | | | |
| D17H6S56E-5 | DNA segment chr 17 human | 2.288E−03 | 1.641 | 1.584E−05 | 2.075 | 6.988E−06 | 2.149 | 1.440E−07 | 2.524 | 2.571E−10 | 3.246 | 1.478E−04 | 1.877 |
| | D6S56E 5 | | | | | | | | | | | | |
| Dcdc5 | Double cortin domain | 2.570E−02 | 1.348 | 3.209E−10 | 2.667 | 2.736E−08 | 2.300 | 9.845E−12 | 2.994 | 1.413E−05 | 1.853 | 1.309E−05 | 1.858 |
| | containing 5 | | | | | | | | | | | | |
| Emr4 | Egf-like module containing, | 1.268E−05 | −1.683 | 5.015E−03 | −1.375 | 1.110E−07 | −1.933 | 2.696E−09 | −2.145 | 7.000E−09 | −2.089 | 7.485E−13 | −2.696 |
| | mucin-like, hormone | | | | | | | | | | | | |
| | receptor-like 4 | | | | | | | | | | | | |
| Fam126b | Family with sequence | 5.669E−03 | −1.385 | 9.889E−04 | −1.481 | 6.428E−04 | −1.505 | 2.287E−05 | −1.684 | 6.527E−04 | −1.504 | 3.062E−04 | −1.544 |
| | similarity 126 member B | | | | | | | | | | | | |
| Gipc2 | Gipc PDZ domain containing | 1.146E−05 | 1.815 | 1.360E−13 | 3.270 | 4.127E−08 | 2.186 | 1.614E−13 | 3.251 | 9.202E−14 | 3.311 | 8.526E−18 | 4.532 |
| | family, member 2 | | | | | | | | | | | | |
| Gm13051 | Predicted gene 13051 | 1.840E−03 | 1.313 | 8.093E−09 | 1.751 | 1.066E−08 | 1.740 | 6.500E−14 | 2.251 | 5.108E−17 | 2.644 | 2.894E−17 | 2.679 |
| Gm13251 | Predicted gene 13251 | 3.456E−03 | 1.421 | 9.459E−08 | 2.015 | 5.042E−09 | 2.198 | 1.104E−14 | 3.238 | 1.153E−17 | 4.021 | 7.801E−18 | 4.072 |
| Gm4738 | Esterase 31-like | 3.909E−03 | −1.849 | 3.370E−12 | −5.922 | 2.705E−15 | −8.679 | 1.865E−19 | −15.05 | 1.566E−20 | −17.52 | 2.763E−25 | −36.257 |
| Gm5631 | Predicted gene 5631 | 1.242E−12 | −2.639 | 8.283E−13 | −2.669 | 3.909E−04 | −1.503 | 1.249E−08 | −2.044 | 5.764E−07 | −1.835 | 1.991E−22 | −5.282 |
| Gp49a | Glycoprotein 49A | 2.370E−02 | 1.505 | 1.273E−09 | 3.539 | 5.367E−15 | 6.215 | 1.004E−18 | 9.455 | 9.392E−21 | 12.072 | 2.965E−25 | 21.871 |
| Gpr137b-ps | G-protein coupled receptor | 1.015E−02 | 1.567 | 3.439E−13 | 4.797 | 1.094E−16 | 6.900 | 1.490E−21 | 12.012 | 4.305E−27 | 24.938 | 3.299E−26 | 22.004 |
| | 137B pseudogene | | | | | | | | | | | | |
| Heatr7a | HEAT repeat containing 7A | 6.539E−09 | 1.424 | 2.890E−10 | 1.484 | 2.518E−08 | 1.398 | 4.366E−10 | 1.476 | 9.147E−08 | 1.374 | 1.158E−12 | 1.598 |
| Ifi27l2b | Interferon alpha-inducible | 8.123E−05 | 1.562 | 2.406E−07 | 1.849 | 2.429E−12 | 2.522 | 1.535E−14 | 2.899 | 1.618E−16 | 3.300 | 2.249E−13 | 2.691 |
| | protein 27 like 2B | | | | | | | | | | | | |
| Keg1 | Kidney expressed gene 1 | 1.232E−03 | −1.933 | 1.932E−07 | −3.135 | 7.247E−07 | −2.930 | 8.281E−10 | −4.114 | 6.666E−10 | −4.158 | 3.898E−15 | −7.617 |
| Mlkl | Mixed lineage kinase | 2.534E−02 | 1.366 | 1.325E−02 | 1.415 | 3.346E−03 | 1.515 | 7.886E−06 | 1.943 | 2.173E−05 | 1.870 | 1.027E−02 | 1.433 |
| | domain-like | | | | | | | | | | | | |
| Mpeg1 | Macrophage expressed gene 1 | 1.720E−06 | 1.750 | 5.323E−12 | 2.470 | 7.668E−15 | 2.957 | 1.959E−19 | 4.037 | 2.339E−24 | 5.887 | 5.908E−25 | 6.186 |
| Mtmr11 | Myotubularin related protein | 2.305E−09 | 2.319 | 1.833E−10 | 2.505 | 2.805E−19 | 4.814 | 3.097E−18 | 4.429 | 1.033E−10 | 2.549 | 5.073E−14 | 3.224 |
| | 11 | | | | | | | | | | | | |
| Tlcd2 | TLC domain containing 2 | 3.013E−02 | −1.322 | 6.395E−03 | −1.427 | 1.975E−02 | −1.352 | 2.090E−09 | −2.426 | 2.158E−09 | −2.424 | 9.053E−14 | −3.355 |
| Tmem65 | Transmembrane protein 65 | 8.780E−06 | 1.493 | 1.471E−10 | 1.891 | 1.846E−08 | 1.708 | 1.041E−16 | 2.569 | 6.995E−16 | 2.461 | 5.412E−21 | 3.254 |
| Tmem116 | Transmembrane protein 116 | 4.146E−08 | 1.735 | 1.225E−12 | 2.192 | 9.282E−04 | 1.358 | 4.753E−06 | 1.555 | 2.404E−04 | 1.409 | 3.671E−07 | 1.651 |
| Uap1l1 | UDP-N-acetylglucosamine | 1.123E−10 | 1.991 | 1.395E−21 | 3.676 | 7.990E−23 | 3.983 | 1.491E−25 | 4.803 | 1.114E−26 | 5.213 | 3.913E−29 | 6.299 |
| | pyrophosphorylase 1-like 1 | | | | | | | | | | | | |
| Wbp5 | WW domain binding protein 5 | 1.520E−03 | 1.497 | 9.473E−04 | 1.526 | 8.208E−03 | 1.394 | 7.209E−04 | 1.542 | 2.783E−02 | 1.315 | 1.125E−02 | 1.374 |

Thirteen of these DEG function in cholesterol and lipid homeostasis. As discussed above, dysregulation of cholesterol homeostatic genes is to be expected in NPC disease. Also among this subset were the genes Pltp and ApoA4, which are regulated by zinc finger protein 202 (ZNF202), and belong to a pathway found altered at five ages (Table 3). Two transcription factors, Jun and Spi-c, were also upregulated at all time points. Jun is one of the transcription factors activated by MAPK signaling, and has a broad spectrum of target genes, regulating cell proliferation or apoptosis, as well as immune and stress responses (Schaeffer and Weber, Mol. Cell Biol. 19:2435-44 (1999)). Conversely, Spi-c is a highly specific transcription factor, only expressed in red pulp macrophages where it regulates their correct differentiation (Kohyama et al., Nature 457:318-21 (2009)).

Aldolase A-encoding gene (Aldoa) was upregulated in mutant mice at all ages (Table 4). Aldolases encode enzymes involved in glycolysis, and are differentially expressed during development. Aldoa is highly expressed in fetal liver, and is rapidly repressed after birth and replaced by aldolase B (Aldob) (Numazaki et al., Eur. J. Biochem. 142:165-70 (1984); and Reid and Masters, Mech. Ageing Dev. 30:299-317 (1985)). Aldoa expression was not decreased and Aldob expression did not increase in Npc1 mutant mice. Altered expression of aldolases in Npc1^{-/-} mice likely reflects an abnormal maturation of liver.

Nine cytochrome P450 genes showed consistent downregulation over the whole time course. These genes encode enzymes involved in arachidonic acid metabolism, particularly the Cyp2c subfamily. In addition to the CYP genes, hematopoietic prostaglandin D synthase (Hpgds), encoding the key enzyme in the synthesis of prostaglandins (Herlong and Scott, Immunol. Lett. 102:121-31 (2006)) was also consistently upregulated. Alterations of these metabolic processes likely impact both the inflammatory response and metabolism of xenobiotics and drugs.

### Example 4

### Identification of Biomarkers

Because of the marked clinical heterogeneity of NPC and lack of defined clinical outcome measures, quantitative biomarkers would be a significant tool in the evaluation of potential therapeutic interventions. They could also provide a serum-based diagnostic test. To identify potential biomarkers, the DEG lists were cross-referenced with human or mouse proteins identified as secreted in the Secreted Protein (http://spd.cbi.pku.edu.cn/) or UniProt databases (http://www.uniprot.org/). This comparison identified 961 genes that were differentially expressed at one or more ages. To reduce this candidate list, genes that were differentially expressed at four or more ages were selected, and confirmed that they were secreted proteins based on literature review (Table 5). This analysis identified 103 DEG as candidate biomarkers, and 17 of these genes were differentially expressed at all six time points.

TABLE 5
List of secreted proteins with a modified expression between Npc1^{-/-} and control
mice, sorted by decreasing number of age with a modified expression. p-values and fold-
changes (FC) are indicated at each age.

| | | 1 week | | 3 weeks | | 5 weeks | | 7 weeks | | weeks | | 11 weeks | |
|---|---|---|---|---|---|---|---|---|---|---|---|---|---|
| Gene | | p- | | p- | | p- | | p- | | p- | | p- | |
| symbol | Gene description | value | FC | value | FC | value | FC | value | FC | value | FC | value | FC |
| Apoa4 | Apolipoprotein A-IV | 3.1E−09 | 2.42 | 6.6E−12 | 2.96 | 2.5E−05 | 1.79 | 8.8E−04 | 1.56 | 5.5E−05 | 1.74 | 2.5E−07 | 2.10 |
| Bglap2 | Bone gamma- | 3.5E−05 | 1.49 | 4.9E−05 | 1.48 | 1.2E−09 | 1.90 | 3.1E−08 | 1.77 | 2.4E−06 | 1.60 | 7.4E−05 | 1.46 |
| | carboxyglutamate | | | | | | | | | | | | |
| | protein 2, or | | | | | | | | | | | | |
| | osteocalcin-2 | | | | | | | | | | | | |
| Bglap-rs1 | Bone gamma- | 1.2E−04 | 2.17 | 9.4E−06 | 2.49 | 1.6E−07 | 3.05 | 5.9E−05 | 2.26 | 5.3E−03 | 1.72 | 6.5E−03 | 1.70 |
| | carboxyglutamate | | | | | | | | | | | | |
| | protein, related | | | | | | | | | | | | |
| | sequence 1 | | | | | | | | | | | | |
| C8g | Complement | 7.0E−12 | −1.75 | 1.5E−11 | −1.72 | 2.5E−06 | −1.41 | 2.9E−12 | −1.77 | 1.4E−11 | −1.73 | 7.5E−23 | −2.79 |
| | component 8 gamma | | | | | | | | | | | | |
| | polypeptide | | | | | | | | | | | | |
| Ctsd | Cathepsin D | 1.0E−05 | 1.37 | 4.0E−13 | 1.83 | 4.1E−17 | 2.15 | 3.5E−24 | 2.97 | 9.7E−25 | 3.06 | 4.0E−27 | 3.47 |
| Dpp7 | Dipeptidyl-peptidase 7 | 4.9E−05 | 1.40 | 1.7E−05 | 1.43 | 2.9E−06 | 1.48 | 1.0E−08 | 1.66 | 1.4E−07 | 1.58 | 3.4E−08 | 1.62 |
| Folr2 | Folate receptor 2 | 1.7E−07 | −1.56 | 4.9E−10 | −1.75 | 4.9E−12 | −1.91 | 2.7E−13 | −2.02 | 8.9E−16 | −2.26 | 5.4E−15 | −2.18 |
| Fuca2 | Fucosidase alpha-L-2 | 1.4E−03 | 1.34 | 2.7E−09 | 1.83 | 1.4E−11 | 2.06 | 2.7E−06 | 1.57 | 1.3E−14 | 2.40 | 8.9E−14 | 2.30 |
| Gpx3 | Glutathione | 2.5E−02 | 1.31 | 1.1E−04 | 1.63 | 1.4E−05 | 1.74 | 1.9E−11 | 2.64 | 4.2E−08 | 2.10 | 1.8E−18 | 4.40 |
| | peroxidase 3 | | | | | | | | | | | | |
| Hexa | Hexosaminidase A | 2.8E−13 | 2.05 | 2.1E−17 | 2.49 | 2.5E−17 | 2.48 | 6.2E−24 | 3.55 | 6.1E−23 | 3.35 | 1.9E−25 | 3.89 |
| | alpha subunit | | | | | | | | | | | | |
| Hexb | Hexosaminidase B | 4.4E−08 | 1.91 | 5.6E−10 | 2.15 | 1.5E−10 | 2.22 | 3.0E−11 | 2.32 | 3.0E−14 | 2.79 | 2.2E−14 | 2.81 |
| | beta subunit | | | | | | | | | | | | |
| Il1rap | Interleukin 1 receptor | 6.7E−04 | −1.32 | 1.4E−06 | −1.52 | 5.3E−05 | −1.40 | 5.9E−09 | −1.69 | 7.4E−08 | −1.61 | 2.8E−17 | −2.50 |
| | accessory protein | | | | | | | | | | | | |
| Inhbc | inhibin/actiyin beta C | 2.1E−07 | −1.83 | 2.6E−03 | −1.38 | 8.4E−03 | −1.33 | 3.1E−06 | −1.70 | 3.3E−08 | −1.93 | 2.1E−18 | −3.66 |
| | chain | | | | | | | | | | | | |
| Lgals1 | Lectin galactoside- | 2.6E−07 | 1.71 | 2.2E−09 | 1.91 | 9.6E−13 | 2.29 | 4.2E−21 | 3.76 | 6.1E−22 | 3.98 | 3.1E−22 | 4.06 |
| | binding soluble 1 | | | | | | | | | | | | |
| Lgals3 | Lectin galactoside- | 2.3E−02 | 1.48 | 2.6E−11 | 3.91 | 1.3E−15 | 6.04 | 2.8E−22 | 12.77 | 1.2E−21 | 11.84 | 7.0E−24 | 15.61 |
| | binding soluble 3, or | | | | | | | | | | | | |
| | macrophage galactose | | | | | | | | | | | | |
| | specific lectin 2 | | | | | | | | | | | | |
| Mfge8 | Milk fat globule-EGF | 9.7E−05 | 1.37 | 4.8E−06 | 1.46 | 4.6E−05 | 1.39 | 1.3E−14 | 2.14 | 2.1E−19 | 2.70 | 1.6E−27 | 4.29 |
| | factor 8 protein, or | | | | | | | | | | | | |
| | lactadherin, or medin | | | | | | | | | | | | |
| Npc2 | Niemann-Pick disease | 2.0E−05 | 1.46 | 1.1E−03 | 1.32 | 1.0E−06 | 1.56 | 3.8E−09 | 1.76 | 1.3E−06 | 1.55 | 1.7E−09 | 1.79 |
| | type C2 | | | | | | | | | | | | |
| Pltp | Phospholipid transfer | 2.3E−03 | −1.35 | 6.8E−03 | 1.30 | 6.4E−10 | 1.99 | 2.1E−10 | 2.05 | 3.1E−14 | 2.53 | 2.7E−17 | 3.03 |
| | protein, or lipid | | | | | | | | | | | | |
| | transfer protein II | | | | | | | | | | | | |
| Prok1 | Prokineticin 1, or | 4.2E−03 | −1.56 | 3.0E−03 | −1.59 | 5.1E−04 | −1.73 | 5.4E−04 | −1.72 | 5.8E−05 | −1.91 | 1.0E−06 | −2.25 |
| | endocrine-gland- | | | | | | | | | | | | |
| | derived vascular | | | | | | | | | | | | |
| | endothelial growth | | | | | | | | | | | | |
| | factor | | | | | | | | | | | | |
| Saa4 | Serum amyloid A4 | 5.0E−02 | −1.32 | 1.5E−10 | −2.89 | 1.1E−05 | −1.94 | 8.1E−07 | −2.14 | 9.1E−09 | −2.51 | 3.1E−18 | −5.54 |
| Afm | Afamin, or alpha- | 4.4E−03 | −1.24 | 1.2E−04 | −1.35 | | | 4.0E−10 | −1.72 | 3.5E−10 | −1.72 | 4.9E−27 | −3.97 |
| | albumin | | | | | | | | | | | | |
| Bgn | Biglycan, or | | | 1.7E−06 | 1.34 | 1.2E−06 | 1.35 | 8.9E−11 | 1.55 | 2.1E−09 | 1.48 | 2.1E−12 | 1.63 |
| | bone/cartilage | | | | | | | | | | | | |
| | proteoglycan-1 | | | | | | | | | | | | |
| C8a | Complement | 1.2E−02 | −1.35 | | | 4.6E−03 | −1.40 | 6.0E−03 | −1.39 | 3.0E−03 | −1.43 | 2.0E−22 | −5.88 |
| | component C8 alpha | | | | | | | | | | | | |
| | chain | | | | | | | | | | | | |
| C9 | Complement | | | 1.9E−07 | −2.46 | 4.3E−05 | −1.96 | 2.2E−04 | −1.82 | 2.0E−04 | −1.83 | 2.5E−19 | −7.44 |
| | component C9 | | | | | | | | | | | | |
| Ccl3 | C-C motif chemokine | | | 4.2E−05 | 1.70 | 3.2E−09 | 2.30 | 1.7E−07 | 2.04 | 1.1E−10 | 2.55 | 6.6E−20 | 5.09 |
| | 3, or Macrophage | | | | | | | | | | | | |
| | inflammatory protein | | | | | | | | | | | | |
| | 1-alpha | | | | | | | | | | | | |
| Ccl5 | C-C motif chemokine | | | 1.3E−04 | 1.46 | 3.5E−04 | 1.42 | 3.2E−04 | 1.43 | 6.8E−08 | 1.77 | 1.7E−05 | 1.54 |
| | 5, or T-cell-specific | | | | | | | | | | | | |
| | protein RANTES | | | | | | | | | | | | |
| Ccl6 | C-C motif | | | 7.1E−05 | 1.57 | 3.1E−07 | 1.83 | 1.1E−11 | 2.42 | 3.4E−13 | 2.65 | 9.2E−19 | 3.83 |
| | chemokine 6 | | | | | | | | | | | | |
| Cd44 | Cd44 antigen | | | 9.9E−08 | 1.48 | 3.0E−10 | 1.63 | 5.6E−22 | 2.64 | 3.7E−19 | 2.33 | 6.1E−26 | 3.22 |
| Cd48 | Cd48 antigen | | | 7.2E−06 | 1.63 | 2.3E−08 | 1.90 | 2.2E−11 | 2.27 | 4.1E−17 | 3.21 | 1.2E−17 | 3.32 |
| Cfh | Complement factor H | 7.8E−04 | −1.33 | 1.1E−06 | −1.55 | | | 4.2E−08 | −1.66 | 4.4E−04 | −1.35 | 7.9E−16 | −2.40 |
| Col6a3 | Collagen alpha-3(VI) | | | 9.3E−05 | 1.39 | 1.7E−07 | 1.60 | 3.2E−08 | 1.65 | 1.5E−08 | 1.68 | 5.4E−05 | 1.41 |
| | chain | | | | | | | | | | | | |
| Ctsb | Cathepsin B, or APP | | | 1.1E−08 | 1.49 | 5.0E−12 | 1.68 | 1.2E−18 | 2.15 | 7.0E−19 | 2.17 | 7.1E−23 | 2.58 |
| | secretase | | | | | | | | | | | | |
| Ctsl | Cathepsin L1, or | 9.6E−06 | 1.33 | | | 1.7E−11 | 1.62 | 1.6E−07 | 1.42 | 1.5E−17 | 2.02 | 3.6E−22 | 2.43 |
| | major excreted protein | | | | | | | | | | | | |
| Ctss | Cathepsin S | | | 7.7E−08 | 2.14 | 1.0E−14 | 3.56 | 1.5E−20 | 5.71 | 1.3E−24 | 8.30 | 2.1E−25 | 8.99 |
| Defb1 | Beta-defensin 1 | | | 1.8E−04 | 1.91 | 3.5E−04 | 1.85 | 4.7E−05 | 2.04 | 3.3E−04 | 1.85 | 3.8E−13 | 4.47 |
| Frzb | Secreted frizzled- | | | 1.5E−03 | 1.31 | 4.3E−06 | 1.51 | 9.5E−11 | 1.90 | 9.5E−14 | 2.19 | 2.0E−14 | 2.27 |
| | related protein 3 | | | | | | | | | | | | |
| Grn | Granulin | | | 7.5E−14 | 1.72 | 1.9E−08 | 1.44 | 4.4E−13 | 1.68 | 2.2E−12 | 1.64 | 3.8E−15 | 1.80 |
| Hpse | Heparanase | | | 7.8E−05 | 1.59 | 1.7E−10 | 2.31 | 1.9E−19 | 4.22 | 6.0E−25 | 6.53 | 4.8E−25 | 6.58 |
| Il18bp | Interleukin-18- | | | 2.7E−04 | 1.43 | 7.7E−04 | 1.39 | 3.3E−07 | 1.71 | 2.4E−08 | 1.82 | 9.1E−12 | 2.19 |
| | binding protein | | | | | | | | | | | | |
| Il1b | Interleukin-1 beta | 8.9E−04 | −1.65 | | | 5.9E−02 | −1.32 | 1.9E−02 | −1.41 | 7.5E−03 | −1.49 | 1.0E−02 | −1.46 |
| Il7r | Interleukin-7 receptor | | | 5.3E−03 | 1.77 | 3.1E−08 | 3.49 | 1.1E−17 | 10.75 | 1.2E−21 | 18.29 | 3.8E−22 | 19.61 |
| | subunit alpha | | | | | | | | | | | | |
| Lgmn | Legumain, or | | | 9.1E−09 | 1.74 | 3.9E−12 | 2.06 | 9.8E−20 | 3.07 | 1.1E−23 | 3.89 | 6.7E−27 | 4.82 |
| | asparaginyl | | | | | | | | | | | | |
| | endopeptidase | | | | | | | | | | | | |
| Lifr | Leukemia inhibitory | | | 1.7E−03 | −1.36 | 1.5E−13 | −2.46 | 6.3E−13 | −2.37 | 2.6E−14 | −2.57 | 1.9E−18 | −3.29 |
| | factor receptor | | | | | | | | | | | | |
| Ltf | Lactotransferrin | 2.3E−03 | −1.36 | | | 6.2E−04 | 1.42 | 1.3E−09 | 1.99 | 2.6E−07 | 1.75 | 1.7E−06 | 1.67 |
| Ly86 | Ly6mphocyte antigen | | | 9.8E−03 | 1.36 | 1.8E−06 | 1.84 | 3.7E−09 | 2.21 | 6.6E−16 | 3.51 | 1.2E−20 | 5.03 |
| | 86 | | | | | | | | | | | | |
| Mmp12 | Matrix | | | 2.4E−14 | 7.13 | 1.9E−19 | 13.58 | 1.9E−23 | 23.91 | 4.4E−24 | 26.27 | 6.0E−22 | 19.18 |
| | metalloproteinase-12, | | | | | | | | | | | | |
| | or macrophage | | | | | | | | | | | | |
| | elastase | | | | | | | | | | | | |
| Pdgfa | Platelet-derived | | | 1.1E−03 | 1.32 | 1.9E−03 | 1.30 | 1.1E−10 | 1.89 | 1.1E−05 | 1.48 | 1.5E−04 | 1.39 |
| | growth factor | | | | | | | | | | | | |
| | subunit A | | | | | | | | | | | | |
| Pdgfb | Platelet-derived | | | 6.0E−04 | 1.31 | 1.9E−04 | 1.35 | 5.8E−09 | 1.66 | 1.6E−09 | 1.70 | 1.6E−11 | 1.85 |
| | growth factor | | | | | | | | | | | | |
| | subunit B | | | | | | | | | | | | |
| Pla2g7 | Platelet-activating | | | 8.1E−03 | 1.34 | 1.3E−07 | 1.91 | 1.8E−12 | 2.60 | 1.6E−18 | 3.90 | 5.1E−22 | 5.07 |
| | factor | | | | | | | | | | | | |
| | acetylhydrolase, or | | | | | | | | | | | | |
| | Group-VIIA | | | | | | | | | | | | |
| | phospholipase A2 | | | | | | | | | | | | |
| Plau | Urokinase-type | | | 8.2E−04 | 1.52 | 2.2E−07 | 2.01 | 5.1E−12 | 2.78 | 4.2E−09 | 2.27 | 2.6E−10 | 2.47 |
| | plasminogen activator | | | | | | | | | | | | |
| Plaur | Urokinase | | | 2.9E−04 | 1.46 | 7.3E−09 | 1.94 | 5.5E−17 | 3.13 | 1.9E−19 | 3.67 | 6.1E−25 | 5.45 |
| | plasminogen activator | | | | | | | | | | | | |
| | surface receptor | | | | | | | | | | | | |
| Ppbp | Pro-platelet basic | | | 3.1E−03 | 1.83 | 3.6E−05 | 2.40 | 2.8E−04 | 2.13 | 1.1E−05 | 2.56 | 2.4E−03 | 1.86 |
| | protein, also Cxcl7 | | | | | | | | | | | | |
| Serpina9 | SerpinA9 | 1.1E−02 | −1.49 | 8.4E−04 | −1.70 | 9.2E−03 | −1.50 | | | 9.8E−03 | −1.50 | 2.7E−02 | −1.41 |
| Smoc2 | SPARC-related | | | 8.7E−06 | 1.32 | 2.4E−06 | 1.34 | 1.1E−07 | 1.40 | 2.0E−09 | 1.49 | 1.7E−12 | 1.65 |
| | modular calcium- | | | | | | | | | | | | |
| | binding protein 2 | | | | | | | | | | | | |
| Sparcl1 | SPARC-like protein | | | 6.9E−08 | 1.63 | 1.4E−06 | 1.53 | 1.5E−14 | 2.24 | 9.3E−14 | 2.15 | 4.3E−19 | 2.82 |
| | 1, or High Endothelial | | | | | | | | | | | | |
| | Venule Protein | | | | | | | | | | | | |
| Svep1 | Sushi, von Willebrand | | | 8.9E−06 | 1.35 | 4.3E−06 | 1.36 | 8.7E−12 | 1.68 | 6.8E−10 | 1.57 | 1.4E−10 | 1.61 |
| | factor type A, EGF | | | | | | | | | | | | |
| | and pentraxin domain- | | | | | | | | | | | | |
| | containing protein 1 | | | | | | | | | | | | |
| Tfrc | Transferrin receptor | | | 3.8E−02 | 1.53 | 1.1E−02 | 1.69 | 3.7E−04 | 2.12 | 5.9E−04 | 2.06 | 6.8E−08 | 3.41 |
| | protein 1 | | | | | | | | | | | | |
| Thbs1 | Thrombospondin I | | | 2.2E−04 | 1.43 | 3.6E−04 | 1.42 | 6.2E−07 | 1.67 | 3.8E−07 | 1.69 | 1.3E−07 | 1.73 |
| Timp2 | Tissue inhibitor of | | | 1.0E−04 | 1.41 | 3.3E−06 | 1.52 | 2.4E−18 | 2.80 | 3.0E−17 | 2.64 | 7.8E−24 | 3.85 |
| | metalloproteinases 2 | | | | | | | | | | | | |
| Timp3 | Tissue inhibitor of | | | 3.0E−04 | 1.48 | 3.9E−04 | 1.47 | 3.2E−08 | 1.91 | 4.8E−06 | 1.67 | 1.4E−12 | 2.49 |
| | metalloproteinases 3 | | | | | | | | | | | | |
| Trem2 | Triggering receptor | | | 5.6E−05 | 1.54 | 1.1E−07 | 1.82 | 1.5E−15 | 2.91 | 1.5E−19 | 3.77 | 2.3E−19 | 3.72 |
| | expressed on myeloid | | | | | | | | | | | | |
| | cells 2 | | | | | | | | | | | | |
| Vegfb | Vascular endothelial | 1.5E−08 | 1.38 | 7.8E−13 | 1.56 | 1.2E−10 | 1.46 | 1.4E−07 | 1.34 | | | 1.1E−06 | 1.30 |
| | growth factor beta | | | | | | | | | | | | |
| A1bg | Alpha-1B- | | | | | 1.9E−04 | −2.75 | 1.2E−06 | −3.97 | 6.5E−07 | −4.13 | 1.0E−08 | −5.43 |
| | glycoprotein | | | | | | | | | | | | |
| Adamts6 | A disintegrin and | | | 1.8E−04 | 1.72 | 2.2E−05 | 1.87 | | | 4.5E−02 | 1.32 | 2.7E−02 | 1.36 |
| | metalloproteinase | | | | | | | | | | | | |
| | with thrombospondin | | | | | | | | | | | | |
| | motifs 6 | | | | | | | | | | | | |
| Adm | adrenomedullin | | | | | 2.5E−02 | 1.40 | 3.7E−03 | 1.56 | 5.8E−03 | 1.52 | 1.7E−07 | 2.39 |
| Alcam | Activated leukocyte | | | | | 2.3E−11 | 1.53 | 3.3E−14 | 1.67 | 7.3E−15 | 1.70 | 1.3E−12 | 1.59 |
| | cell adhesion | | | | | | | | | | | | |
| | molecule | | | | | | | | | | | | |
| C1qa | Complement C1q | | | | | 1.2E−04 | 1.40 | 1.3E−04 | 1.40 | 4.8E−07 | 1.59 | 2.1E−10 | 1.87 |
| | subcomponent | | | | | | | | | | | | |
| | subunit A | | | | | | | | | | | | |
| C1qb | Complement C1q | | | | | 1.5E−03 | 1.34 | 7.3E−05 | 1.46 | 2.9E−03 | 1.32 | 5.5E−14 | 2.36 |
| | subcomponent | | | | | | | | | | | | |
| | subunit B | | | | | | | | | | | | |
| C8b | Complement | | | | | 1.9E−03 | −1.41 | 6.7E−04 | −1.47 | 1.3E−02 | −1.31 | 1.5E−10 | −2.28 |
| | component C8 beta | | | | | | | | | | | | |
| | chain | | | | | | | | | | | | |
| Ccl24 | C-C motif chemokine | | | | | 2.7E−06 | −1.76 | 2.3E−07 | −1.89 | 3.4E−09 | −2.13 | 5.1E−08 | −1.97 |
| | 24, or eotaxin-2 | | | | | | | | | | | | |
| Cd14 | Monocyte | | | | | 2.8E−02 | 1.48 | 8.3E−03 | 1.60 | 2.9E−02 | 1.47 | 1.1E−07 | 2.84 |
| | differentiation antigen | | | | | | | | | | | | |
| | CD14 | | | | | | | | | | | | |
| Colla1 | Collagen alpha-1(1) | | | 1.3E−06 | 1.49 | 1.1E−06 | 1.50 | 6.1E−12 | 1.88 | 2.6E−05 | 1.40 | | |
| | chain | | | | | | | | | | | | |
| Colla2 | Collagen alpha-2(I) | | | 1.2E−05 | 1.40 | 1.5E−05 | 1.39 | 1.0E−08 | 1.59 | 1.4E−06 | 1.45 | | |
| | chain | | | | | | | | | | | | |
| Csf3r | Granulocyte colony- | | | | | 1.0E−03 | 1.33 | 5.2E−10 | 1.85 | 7.2E−12 | 2.02 | 2.3E−10 | 1.88 |
| | stimulating factor | | | | | | | | | | | | |
| | receptor | | | | | | | | | | | | |
| Cxcl10 | C—X—C motif | | | 1.7E−06 | 2.57 | 1.6E−05 | 2.31 | 9.2E−06 | 2.37 | 1.1E−04 | 2.09 | | |
| | chemokine 10 | | | | | | | | | | | | |
| Cxcl16 | C—X—X motif | | | | | 8.1E−05 | 1.52 | 6.9E−08 | 1.84 | 4.8E−13 | 2.50 | 5.2E−10 | 2.09 |
| | chemokine 16 | | | | | | | | | | | | |
| Dcn | Decorin | | | | | 9.2E−06 | 1.34 | 8.7E−07 | 1.39 | 1.8E−12 | 1.70 | 4.3E−16 | 1.94 |
| F13a1 | Coagulation factor | | | 8.1E−04 | 1.36 | | | 2.8E−06 | 1.57 | 1.9E−06 | 1.59 | 2.7E−05 | 1.49 |
| | XIII A chain | | | | | | | | | | | | |
| F9 | Coagulation factor IX | | | 9.4E−04 | −1.32 | | | 2.4E−06 | −1.52 | 5.5E−05 | −1.42 | 2.3E−09 | −1.75 |
| Fga | Fibrinogen alpha | | | 1.0E−04 | −1.97 | | | 3.9E−05 | −2.07 | 1.1E−02 | −1.53 | 2.1E−06 | −2.36 |
| | chain | | | | | | | | | | | | |
| Gdf15 | Growth/differentiation | 5.2E−03 | 1.47 | | | 4.9E−03 | 1.48 | | | 4.2E−04 | 1.65 | 1.9E−02 | 1.38 |
| | factor 15 | | | | | | | | | | | | |
| Hyal1 | Hyaluronidase-1 | | | 3.3E−07 | −1.86 | 2.8E−07 | −1.87 | 6.9E−03 | −1.35 | 1.1E−03 | −1.45 | | |
| Igf1 | Insulin-like growth | | | | | 8.1E−05 | −1.43 | 1.3E−07 | −1.65 | 4.3E−05 | −1.45 | 1.2E−10 | −1.92 |
| | factor I, or | | | | | | | | | | | | |
| | Somatomedin-C | | | | | | | | | | | | |
| Lcn2 | Neutrophil gelatinase- | | | | | 2.2E−02 | 1.93 | 3.3E−05 | 3.52 | 1.5E−06 | 4.46 | 1.7E−07 | 5.25 |
| | associated lipocalin | | | | | | | | | | | | |
| Leap2 | Liver-expressed | | | 1.7E−07 | −2.06 | | | 3.2E−04 | −1.59 | 1.4E−02 | −1.36 | 2.2E−13 | −3.14 |
| | antimicrobial peptide 2 | | | | | | | | | | | | |
| Lect2 | Leukocyte cell- | 2.6E−03 | −1.36 | | | | | 2.3E−04 | −1.47 | 3.3E−03 | −1.35 | 7.2E−11 | −2.18 |
| | derived chemotaxin-2 | | | | | | | | | | | | |
| Loxl2 | Lysyl oxidase | 4.3E−05 | 1.39 | | | 8.3E−04 | 1.30 | 4.7E−05 | 1.39 | | | 5.4E−04 | 1.32 |
| | homolog 2 | | | | | | | | | | | | |
| Lpl | Lipoprotein lipase | | | | | 1.8E−15 | 4.66 | 3.5E−23 | 9.91 | 1.1E−25 | 13.13 | 4.0E−27 | 15.62 |
| Mbl1 | Mannose-binding | | | | | 8.1E−05 | −1.54 | 2.8E−05 | −1.59 | 1.2E−04 | −1.52 | 6.8E−15 | −2.85 |
| | protein A | | | | | | | | | | | | |
| Mif | Macrophage | | | 1.0E−08 | 1.50 | 1.9E−06 | 1.38 | 1.5E−05 | 1.33 | | | 1.9E−07 | 1.43 |
| | migration inhibitory | | | | | | | | | | | | |
| | factor | | | | | | | | | | | | |
| Mmp2 | Matrix | | | | | 3.5E−04 | 1.32 | 5.5E−07 | 1.51 | 1.9E−04 | 1.34 | 8.5E−08 | 1.57 |
| | metalloproteinase-2 | | | | | | | | | | | | |
| Nid1 | Nidogen-1, or entactin | | | | | 2.6E−08 | 1.63 | 1.9E−14 | 2.15 | 2.3E−12 | 1.96 | 1.1E−18 | 2.64 |
| Nrp1 | Neuropilin-1 | | | | | 2.7E−06 | −1.73 | 3.3E−09 | −2.08 | 1.9E−09 | −2.11 | 1.8E−12 | −2.54 |
| Plxnb1 | Plexin-B1 | | | 2.4E−06 | 1.48 | | | 6.2E−11 | 1.82 | 1.4E−05 | 1.43 | 6.1E−05 | 1.38 |
| Postn | Periostin, or | | | | | 2.0E−03 | 1.44 | 1.4E−04 | 1.58 | 9.2E−07 | 1.84 | 2.8E−08 | 2.04 |
| | Osteoblast-specific | | | | | | | | | | | | |
| | factor 2 | | | | | | | | | | | | |
| Pvr | Poliovirus receptor, or | | | 2.7E−04 | 1.50 | | | 1.5E−05 | 1.64 | 1.0E−03 | 1.44 | 4.0E−05 | 1.59 |
| | Cd155 | | | | | | | | | | | | |
| Selplg | P-selectin | | | 4.5E−04 | 1.32 | | | 2.1E−06 | 1.49 | 5.4E−13 | 2.00 | 1.5E−09 | 1.72 |
| | glycoprotein ligand 1 | | | | | | | | | | | | |
| Serpina12 | Serpin A12, or | | | 1.2E−03 | −1.52 | | | 1.0E−03 | −1.53 | 8.8E−03 | −1.39 | 4.3E−03 | −1.44 |
| | Visceral adipose | | | | | | | | | | | | |
| | tissue-derived serine | | | | | | | | | | | | |
| | protease inhibitor | | | | | | | | | | | | |
| Slpi | Antileukoproteinase, | 4.8E−05 | 1.42 | | | | | 2.2E−06 | 1.52 | 4.9E−06 | 1.50 | 1.2E−10 | 1.87 |
| | or Secretory leukocyte | | | | | | | | | | | | |
| | protease inhibitor | | | | | | | | | | | | |
| Tfpi2 | Tissue factor pathway | | | 1.2E−03 | −1.38 | | | 2.0E−07 | −1.73 | 8.9E−06 | −1.57 | 1.7E−17 | −3.05 |
| | inhibitor 2 | | | | | | | | | | | | |
| Tgfbi | Transforming growth | | | 3.3E−04 | 1.34 | | | 5.9E−08 | 1.61 | 5.6E−10 | 1.76 | 6.4E−07 | 1.54 |
| | factor-beta-induced | | | | | | | | | | | | |
| | protein ig-h3 | | | | | | | | | | | | |
| Thbs2 | Thrombospondin-2 | | | | | 5.6E−04 | 1.34 | 4.7E−05 | 1.42 | 1.0E−06 | 1.55 | 8.7E−08 | 1.63 |
| Tnxb | Tenascin-X | | | 4.2E−06 | 1.61 | | | 2.9E−04 | 1.43 | 7.9E−05 | 1.49 | 1.3E−06 | 1.65 |

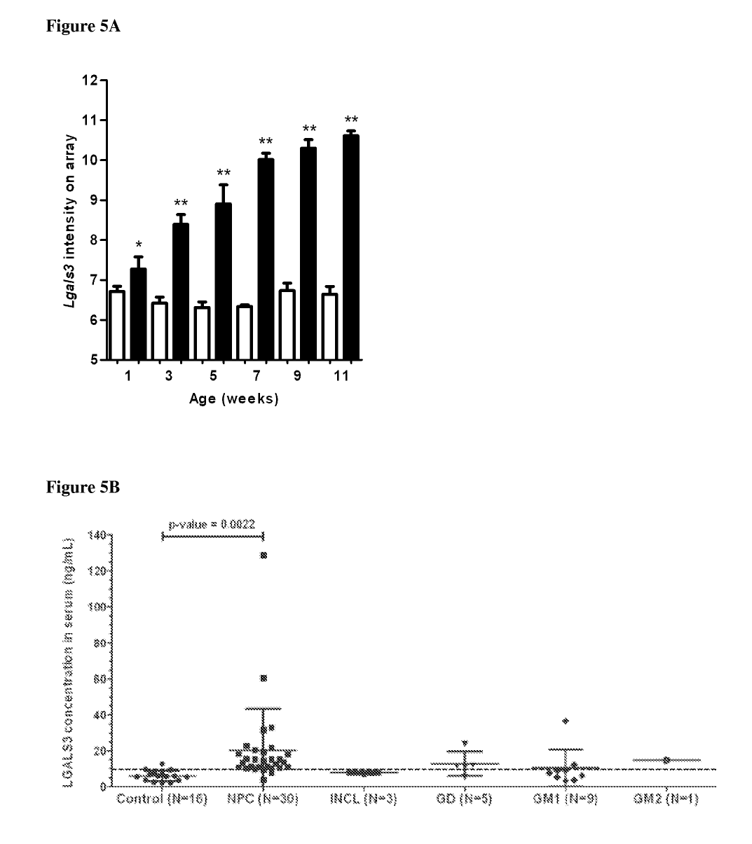

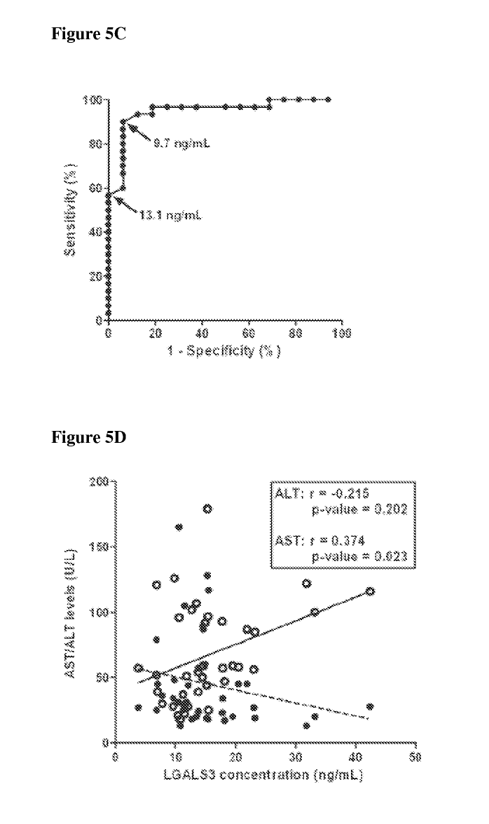

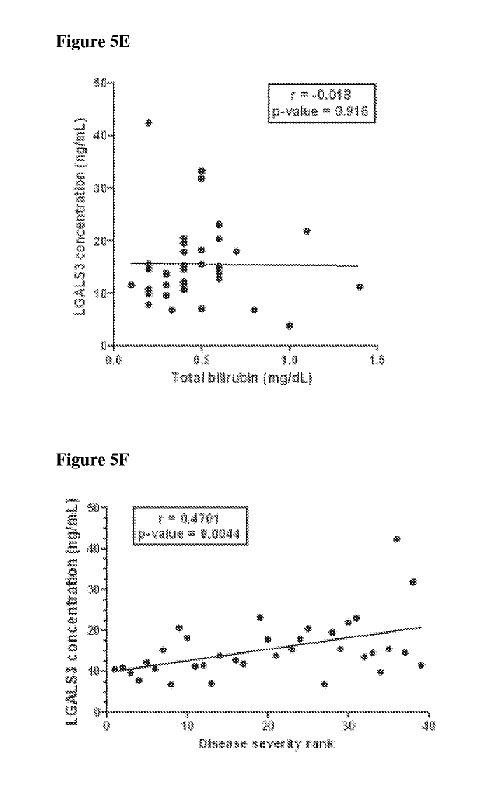

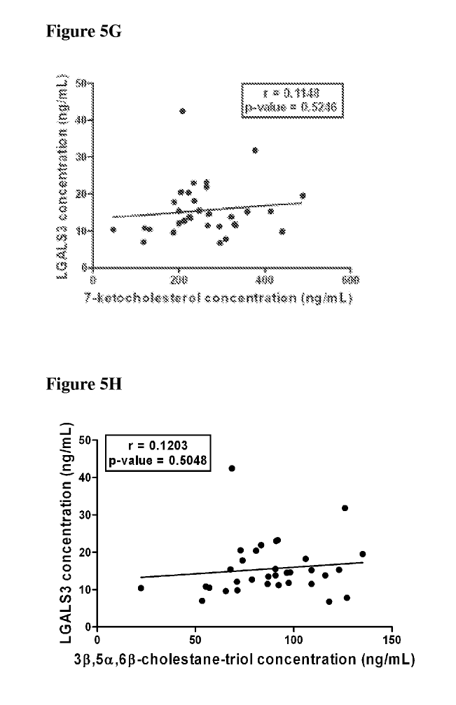

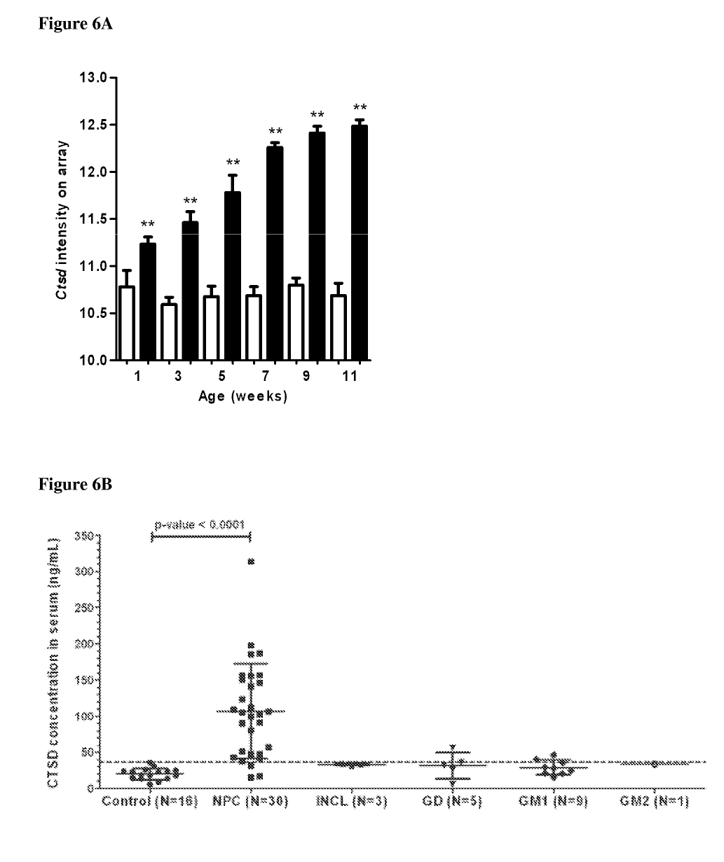

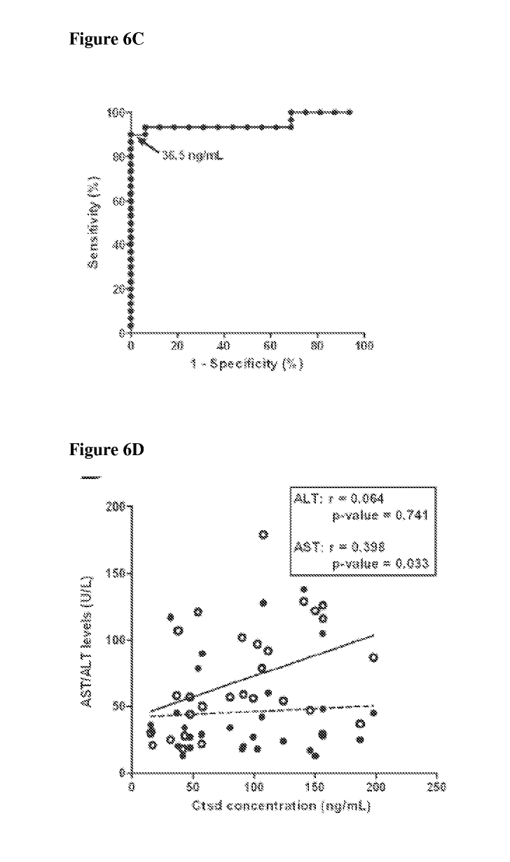

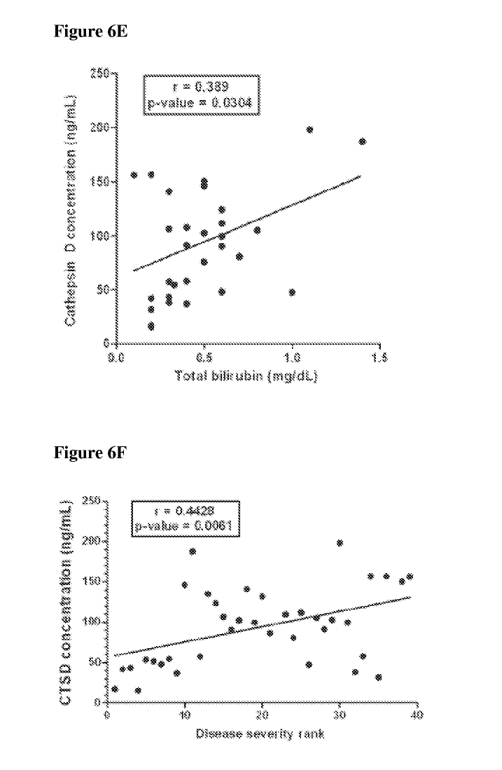

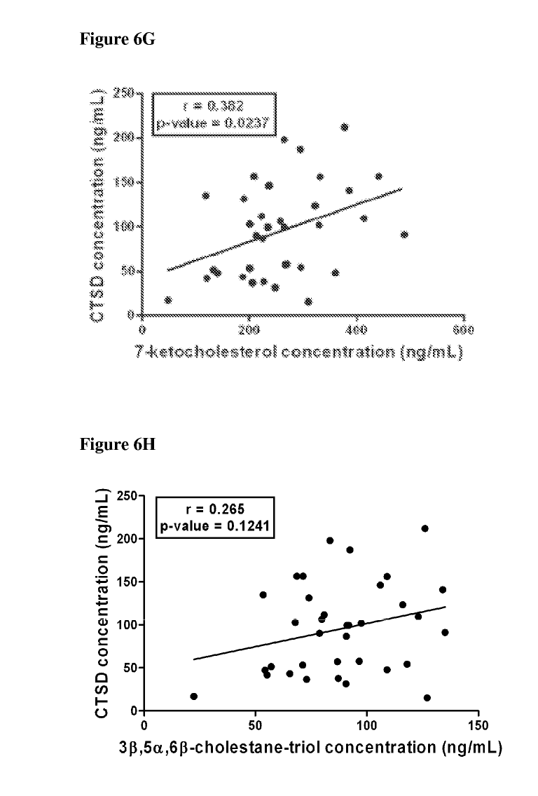

To determine whether this approach could be used to identify potential serum biomarkers, the expression of plasminogen activator urokinase-type (PLAU), lectin galactoside-binding soluble 3 (LGALS3, also called galectin-3, or macrophage galactose-specific lectin or mac-2) and cathepsin D (CTSD) were quantified in NPC1 patients' serum. These three genes were selected because their expression was altered at five or six ages, and because they demonstrated increased dysregulation with age (Table 5 and FIGS. 5A, 6A, and 7A). Explorative testing using sera from six NPC1 patients and six age-matched controls showed markedly different levels between the two groups for LGALS3 and CTSD, but not PLAU. LGALS3 and CTSD levels were therefore determined for twenty-four additional NPC1 patients and ten additional controls. Serum levels of both LGALS3 and CTSD were significantly increased in NPC1 patients compared to age appropriate controls (FIGS. 5B and 6B). Mean serum LGALS3 levels were 6.0±2.9 ng/mL (range: 2.2 to 12.7) and 20.3±23.0 ng/mL (range: 3.8 to 128.8) for controls and NPC1 patients, respectively (P-value ≦0.005). Mean serum CTSD levels were 19.9±7.8 ng/mL (range: 5.6 to 35.1) and 106.7±65.7 ng/mL (range: 15.3 to 314) for controls and NPC1 patients respectively (P-value ≦0.0001). Given the variability of both protein concentrations in NPC sera, a Grubb's test was performed to identify outliers. The patient with the highest CTSD value, as well as the two patients with the highest LGALS3 values, were identified as outliers with 95% confidence, and were therefore not considered for subsequent analyses.

To determine if elevated serum levels of LGALS3 and CTSD were specific to NPC or a general finding associated with lysosomal storage diseases (LSD), LGALS3 and CTSD levels were measured in 18 patients with either infantile neuronal ceroid lipofuscinosis (INCL), Gaucher disease (GD), GM1 gangliosidosis, or GM2 gangliosidosis (FIGS. 5B and 6B). Three patients, one affected with GD and two with GM1, had high levels of LGALS3, and intermediate levels of CTSD. Mean concentrations of LGALS3 and CTSD of patients affected with LSD were elevated compared to the mean concentration of the control group, but lower than NPC1 patients' mean levels.

To assess the ability of LGALS3 and CTSD serum concentrations to discriminate NPC1 patients from controls, receiver-operator characteristic (ROC) analysis was performed. ROC curves demonstrated that the area under the curve was 0.9479 and 0.9521 for LGALS3 and CTSD, respectively (FIGS. 5C and 6C). The LGALS3 ROC curve reflected the sizeable overlap between NPC 1 patients' and controls' concentrations. A cut-off value of 13.1 ng/mL would yield a specificity of 100% but a low sensitivity of 56.7%. To get a better sensitivity of 90%, the cut-off value would need to be set at 9.7 ng/mL, yielding a specificity of 93.75% (FIG. 5C). For CTSD, a cut-off value of 36.5 ng/mL yields a sensitivity of 90% and a specificity of 100% (FIG. 6C).

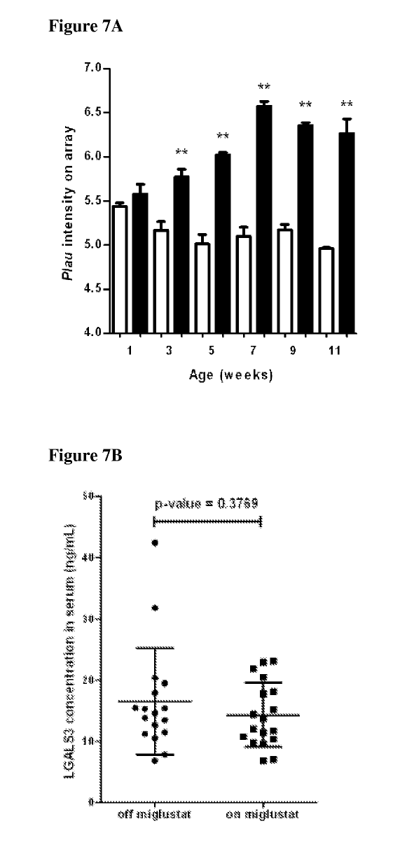

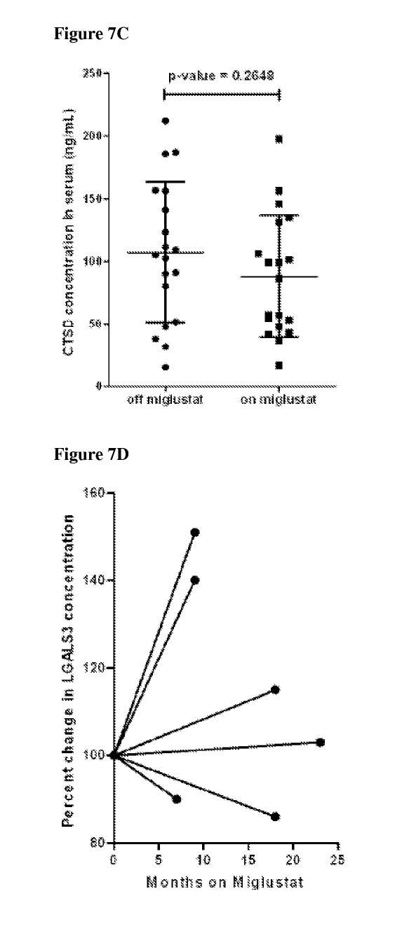

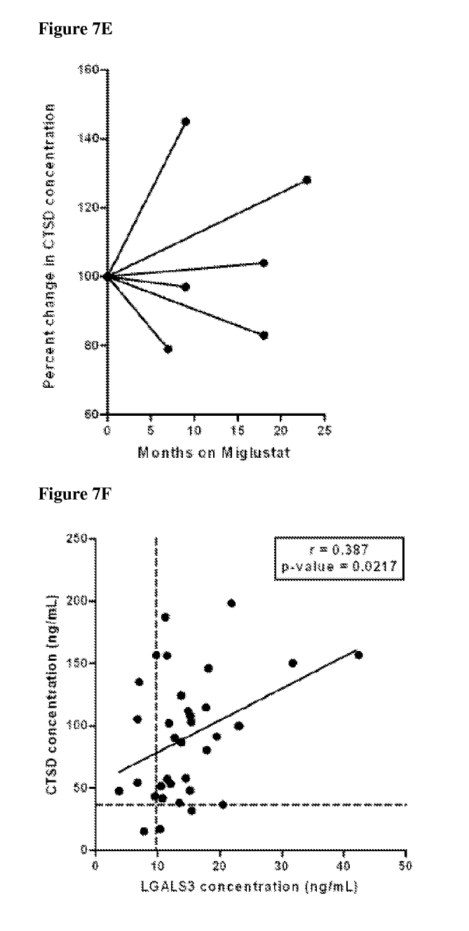

Since 60% of the patients were being treated with off-label miglustat, the effect of treatment on LGALS3 or CTSD concentrations were investigated. No statistical difference was observed between the concentrations of the patients in the two groups (FIGS. 7B and 7C). Since individual responses could be masked by the large degree of variation between patients, LGALS3 and CTSD concentrations were also assessed in serum samples from six patients before and after they begin the treatment. Miglustat treatment did not have any effect on both markers' levels (FIGS. 7D and 7E).

Given the phenotypic heterogeneity observed in NPC, the correlation between serum CTSD and LGALS3 concentrations with disease status was evaluated. As liver function tests are often elevated in NPC patients, the correlation between LGALS3 and CTSD serum levels and liver disease were examined A weak correlation was observed between aspartate aminotransferase (AST) levels and both LGALS3 (r=0.37, P-value ≦0.05) and CTSD (r=0.40, P-value ≦0.05) serum levels, but not between alanine aminotransferase (ALT) and LGALS3 or CTSD concentrations (FIGS. 5D and 6D). Total bilirubin levels were also weakly correlated to CTSD serum concentrations (r=0.39, P-value ≦0.05), but not to LGALS3 concentrations (r=−0.02, P-value=0.92) (FIGS. 5E and 6E). Neurodegeneration is particularly severe in most NPC patients, and evaluated using a severity scale (Yanjanin et al., Am. J. Med. Genet. B. Neuropsychiatr. Genet. 153B:132-40 (2010)). A significant correlation was found between LGALS3 (r=0.47, P-value ≦0.005) and CTSD (r=0.44, P-value ≦0.007) serum levels and increased neurological disease severity (FIGS. 5F and 6F).

Recently, plasma levels of the cholesterol oxidation products 7-ketocholesterol and 3β,5α,6β-cholestane-triol were shown to be specifically elevated in NPC patients (Porter et al., Sci. Transl. Med. 2:56ra81 (2010); and Jiang et al., J. Lipid Res. 52:1435-45 (2011)). To determine if the same pathological processes led to elevations of CTSD, LGALS3 and oxysterols, the correlation of LGALS3 and CTSD concentrations together, or with 7-ketocholesterol and 3β,5α,6β-cholestane-triol levels were evaluated. LGALS3 and CTSD concentrations were weakly correlated (r=0.387, P-value ≦0.05) (FIG. 7F). LGALS3 did not show any correlation with either oxysterol (FIGS. 5G and 5H). CTSD levels were slightly correlated with 7-ketocholesterol concentrations (r=0.382, P-value ≦0.05), but not with 3β,5α,6β-cholestane-triol (FIGS. 6G and 6H).

By comparing global gene expression in postnatal livers from control and Npc1^{-/-} mice, the results described herein identify differences between these two genotypes as early as 1 week of age, well before this model manifests hepatic symptoms. Functional pathways analysis of DEG at each time point identified dysregulated pathways consistent with the general knowledge of NPC disease progression both in mouse and human, as well as new, unexpected pathways. Early changes included deregulation of metabolic pathways, especially related to cholesterol and lipid metabolism, as well as pathways involved in immune response and inflammation, and cell cycle regulation. Post-symptomatic changes (beginning at five weeks of age) involved additional pathways in these functional categories, as well as an increasing number of developmental signaling pathways, especially TGFβ signaling. Still later, upregulation of genes linked to apoptosis and oxidative stress regulation occurred. The study of genes deregulated over the entire time course particularly highlighted MAPK signaling and the transcription factor Jun, as well as the arachidonic acid cascade. Importantly, this study also identified cathepsin D and galectin-3 as new biomarkers for NPC disease, from the DEG in Npc1^{-/-} mice encoding known secreted proteins.

Three microarray studies of NPC tissues have previously been reported: two were conducted using NPC patient and control fibroblasts, and one using cerebellum from three-week-old Npc1^{-/-} and control mice (Reddy et al., PLoS One 1:e19 (2006); Liao et al., Brain Res. 1325:128-40 (2010); and De Windt et al., DNA Cell Biol. 26:665-71 (2007)). While the study by Reddy et al. identified a high proportion of upregulated genes (83%) (Reddy et al., PLoS One 1:e19 (2006)), the results described herein are more in agreement with the studies of De Windt et al. and Liao et al. (Liao et al., Brain Res. 1325:128-40 (2010); and De Windt et al., DNA Cell Biol. 26:665-71 (2007)), with approximately the same number of upregulated and downregulated genes. Variation in the type of tissues, arrays and DEG selection criteria used might explain this difference. Overall comparison of the DEG lists from these three studies with the data described herein showed a good concordance in the DEG among the four microarray results, considering the different cell types and chips used: approximately 25% of DEG from the studies on human fibroblasts, and about 35% of DEG from the mouse cerebella study were modified in the same direction in the dataset. These numbers are likely to be underestimated for the two studies on human fibroblasts, as a murine ortholog was not always identified for these DEG.

The validation of the expression level of 18 DEG by qPCR confirmed the modifications observed on the arrays for all 18 genes. 14 of these genes were previously found to have altered expression in NPC (Reddy et al., PLoS One 1:e19 (2006); Liao et al., Brain Res. 1325:128-40 (2010); and Langmade et al., Proc. Natl. Acad. Sci. USA 103:13807-12 (2006)). However, four of them were previously described with a modified expression in an opposite direction compared to the above results (Cyp51, Idi1, Sqle, and Abcg1) (Liao et al., Brain Res. 1325:128-40 (2010)). The differences in the tissue analyzed may explain these expression differences, as the previous study worked with mouse cerebellum.

Linoleic acid metabolism was identified in the pathway analysis as modified in Npc1^{-/-} mice, with primarily dowregulation of multiple cytochrome P450-encoding genes involved in this pathway. The cytochrome P450 family of enzymes metabolizes therapeutic drugs, as well as endogenous compounds including linoleic and arachidonic acids (Chen and Goldstein, Curr. Drug Metab. 10:567-78 (2009)). The expression of this family of enzymes is mostly induced after birth, during the acquisition of the drug-metabolizing function of the liver (Hines, J. Biochem. Mol. Toxicol. 21:169-75 (2007); and Hart et al., Drug Metab. Dispos. 37:116-21 (2009)). The downregulation of cytochrome P450-encoding genes, as well as the upregulation of aldolase A, normally only expressed in fetal liver (Numazaki et al., Eur. J. Biochem. 142:165-70 (1984); and Reid and Masters, Mech. Ageing Dev. 30:299-317 (1985)), likely underlie a delayed maturation of the liver, which might be related to the transient jaundice seen in NPC. Arachidonic acid and prostaglandin E2 synthesis have recently been shown as increased in NPC cells (Nakamura et al., J. Cell Physiol. (epub 2011)). In addition to delayed liver development, the early downregulation of expression of numerous cytochromes of the Cyp2c subfamily, and the upregulation of expression of a few enzymes in the prostaglandin synthesis cascade could be involved in the increased inflammation observed in NPC disease (Huwiler and Pfeilschifter, Pharmacol. Ther. 124: 96-112 (2009); Kaspera and Totah, Expert Opin. Drug Metab. Toxicol. 5:757-71 (2009); and Node et al., Science 285: 1276-9 (1999)). Cytochrome P450 downregulation is also a significant pharmacogenetic finding, since impaired P450 activity likely results in altered drug metabolism by NPC patients and thus require alteration in medication dosing.

In addition to arachidonic acid metabolism, other pro-inflammatory molecules and signaling have been identified as modified in Npc1^{-/-} mouse liver, especially IL-1 and complement pathways. It has now been discovered that Lgals3, known for its chemoattractant role in acute and chronic inflammation, has increased expression in Npc1^{-/-} mouse liver, and elevated serum levels in NPC patients. Macrophages are the main type of cells secreting galectin-3, and the increased number of Kupffer cells in NPC is likely a source of galectin-3 in serum (Liu et al., Am. J. Pathol. 147:1016-28 (1995)). Alternatively, Lgals3 has also been implicated in fibrotic conditions, and hepatocytes have been shown to express galectin-3 during fibrosis (Hsu et al., Int. J. Cancer 81:519-26 (1999); and Henderson and Sethi, Immunol. Rev. 230:160-71 (2009)). Liver fibrosis sometimes occurs in NPC patients, and was previously described in Npc1^{-/-} mice fed with a high-cholesterol diet, in the mouse antisense-induced NPC1 model and in the feline model (Kelly et al., J. Pediatr. 123:242-7 (1993); Erickson et al., Am. J. Physiol. Gastrointest. Liver Physiol. 289:G300-7 (2005); Rimkunas et al., Hepatology 47:1504-12 (2008); and Somers et al., J. Inherit. Metab. Dis. 24: 427-36 (2001)). Upregulation of Lgals3 as well as TGFβ signaling, one of the main regulators of epithelial-to-mesenchymal transition (EMT) during fibrosis (Wynn, J. Pathol. 214:199-210 (2008); and Lee et al., J. Cell Biol. 172:973-81 (2006)), is likely related to the liver injury observed in NPC disease, and to the numerous modifications in cell adhesion and cytoskeleton remodeling pathways identified in Npc1^{-/-} mice.

MAPK signaling, including the transcription factor Jun, was identified as upregulated in the mutant mice from one week of age onward. Members of the MAPK pathway are ubiquitously expressed, and activated by various types of stimuli, including cytokines, growth hormones, antigens, drugs, adherence to extracellular matrix and cell-cell interactions (Schaeffer and Weber, Mol. Cell Biol. 19:2435-44 (1999)). The specific response to these stimuli will depend on the cellular context. Signaling via Jun is more specifically involved in response to cellular stresses and cytokines (Schaeffer and Weber, Mol. Cell Biol. 19:2435-44 (1999); and Kim and Choi, Biochim. Biophys. Acta 1802:396-405 (2010)). Its upregulation in Npc1^{-/-} mice is likely related to early inflammatory events, e.g., via deregulation in IL-1 signaling, which is a known regulator of MAPK pathway (Weston and Davis, Curr. Opin. Cell Biol. 19:142-9 (2007)).

Spi-c, a PU.1-related transcription factor, was also identified as upregulated in Npc1^{-/-} mice at all six ages. It is mostly expressed in spleen and specifically in red pulp macrophages (RPM), and known to control the development of RPM required for red blood cell recycling and iron homeostasis (Kohyama et al., Nature 457:318-21 (2009)). The finding of Spi-c as one of the genes with a modified expression in Npc1^{-/-} liver is therefore surprising. This result could arise from the abnormal presence of RPM in liver, or alternatively reflect abnormally high expression of this transcription factor in hepatic macrophages or immune cells.

In addition, the study identified novel biomarkers for NPC disease. It was discovered that NPC patients had higher serological concentrations of two secreted proteins with modified expression in the mouse model, LGALS3 and CTSD. High serum LGALS3 levels are likely related to hepatic inflammation, and might participate in the fibrosis sometimes associated with NPC.

The biomarker CTSD is a lysosomal aspartic protease, produced as a pre-pro-protein and processed in the endoplasmic reticulum and lysosomes to produce the active 48 kDa form (Benes and Fusek, Crit. Rev. Oncol. Hematol. 68:12-28 (2008)). Pro-cathepsin D, the secreted, catalytically inactive form, has been shown to act as an autocrine growth factor in various types of cancer (Benes and Fusek, Crit. Rev. Oncol. Hematol. 68:12-28 (2008)). The active enzyme has mostly been implicated in nonspecific protein degradation in the acidic lysosomes. Interestingly, CTSD has been associated with Alzheimer disease (AD): it is abundantly present in senile plaques and neurofibrillary tangles, shows increased levels in the cerebrospinal fluid of AD patients, and is involved in the processing of amyloid precursor protein (APP), Apolipoprotein E (ApoE) and Tau protein (Cataldo and Nixon, Proc. Natl. Acad. Sci. USA 87:3861-5 (1990); Cataldo et al., Neuron 14:671-80 (1995); Schwagerl et al., J. Neurochem. 64:443-6 (1995); Ladror et al., J. Biol. Chem. 269: 18422-8 (1994); Kenessey et al., J. Neurochem. 69:2026-38 (1997); and Zhou et al., Neuroscience 143:689-701 (2006)). The implication of CTSD in AD neurodegeneration prompted studies of this enzyme in NPC disease. In the brain, early increased expression of both the pro-protein and the active enzyme forms of Ctsd was shown in Npc1^{-/-} mice (Liao et al., Am. J. Pathol. 171:962-75 (2007)), and increased Ctsd expression was shown in both the lysosomal and cytosolic compartments of Npc1^{-/-} cerebellum (Amritraj et al., Am. J. Pathol. 175:2540-56 (2009)). In the liver, the upregulation of Ctsd in Npc1^{-/-} hepatocytes was shown to cause increased Abca1 expression (Wang et al., J. Biol. Chem. 282:22525-33 (2007)). The presence of catalytically active Ctsd in the cytosol of NPC cells, as a result of lysosomal permeabilization, may participate in apoptosis. The enzymatic function of CTSD has recently been shown to persist at more neutral pH, and could be responsible for ceramide-induced apoptosis via the processing of BH3-interacting domain death agonist protein, a proapoptotic Bcl-2 family member, and cytochrome c release from mitochondria (Heinrich et al., Cell Death Differ. 11:550-63 (2004)). In addition to NPC disease, cathepsin D had been shown to be upregulated in other LSD, especially in brain homogenates of mouse models for Gaucher Disease, GM1 and GM2 gangliosidosis (Vitner et al., Hum. Mol. Genet. 19:3583-90 (2010)). Although intracellular upregulation of CTSD may be common to LSD, surprising much higher CTSD levels were discovered in NPC patient serum as compared to other LSD patient serum, indicating that increased secretion of this enzyme is a distinctive characteristic of NPC disease.

The findings described herein have great clinical significance as there is an urgent need for improved methods for diagnosing and monitoring NPC. Filipin staining in fibroblasts is currently the standard diagnostic method for NPC disease identification (Wraith et al., Mol. Genet. Metab. 98:152-65 (2009)). To date, the lack of a non-invasive diagnostic test contributes to a delay in NPC diagnosis. Miglustat, not yet FDA-approved but approved for NPC treatment in Europe, has shown efficacy in slowing the neurological progression of the disease (Patterson et al., Lancet Neurol. 6:765-72 (2007)). Therefore, identification of affected patients before the onset of neurological symptoms is critical for beginning effective treatment. The recent identification of 7-ketocholesterol and 3β,5α,6β-cholestane-triol as NPC blood-based biomarkers (Porter et al., Sci. Transl. Med. 2:56ra81 (2010); and Jiang et al., J. Lipid Res. 52:1435-45 (2011)), and the discovery of LGALS3 and CTSD described herein will be used for rapid and non-invasive screening of patients presenting with NPC-suggestive symptoms, such as jaundice in neonates, and neurological deficits or learning disorder in children. The biomarkers will also be used to monitor and identify novel therapies for treating NPC patients. Accordingly, the present invention will improve the therapeutic outcome and quality of life in patients.

The results reported herein were obtained using the following methods and materials.

### Animal Breeding and Tissue Collection

All animal work conformed to NIH guidelines and was approved by the NICHD Institutional Animal Care and Use Committee. Heterozygous Npc1^{+/-} mice were intercrossed to obtain control (Npc1^{+/+}) and mutant (Npc1^{-/-}) littermates. Pups were weaned 3 weeks after birth and subsequently had free access to water and normal mouse chow. PCR genotyping was performed using tail DNA as described in Loftus et al., Science 277:232-5 (1997)). Considering that the phenotypic presentation is slightly different between males and females, only females were evaluated to avoid any gender-specific variations in gene expression (Li, et al., J. Neuropathol. Exp. Neurol. 64:323-33 (2005)). Female pups were sacrificed at 1, 3, 5, 7, 9, and 11 weeks of age. Livers were collected from both mutant and control animals, and immediately frozen on dry ice. Four livers were collected corresponding to each age and genotype, for a total of 48 samples.

### RNA Extraction

Total RNA was extracted from the liver tissue using TRIzol reagent (Invitrogen, Carlsbad, Calif.), followed by purification with Qiagen RNeasy Mini columns (Qiagen, Valencia, Calif.). RNA quality and quantity was assessed using both a Bioanalyzer (Agilent Inc., Santa Clara, Calif.) and NanoDrop (Thermo Scientific Inc., Waltham, Mass.)

### Microarray Hybridization and Data Analysis

Microarray experiments were performed using standard Affymetrix protocols (Affymetrix Inc., Santa Clara, Calif.). Briefly, 200 ng of total RNA was reverse transcribed to obtain labeled cDNA as recommended by the manufacturer. The hybridization cocktail containing the fragmented and labeled cDNAs was hybridized to Affymetrix Mouse GeneChip 1.0 ST chips, and the chips were washed and stained using standard protocols for the Affymetrix Fluidics Station. Probe arrays were stained with streptavidin phycoerythrin solution (Molecular Probes, Carlsbad, Calif.) and enhanced by using an antibody solution containing 0.5 mg/ml of biotinylated anti-streptavidin (Vector Laboratories, Burlingame, Calif.). Arrays were scanned using the Affymetrix Gene Chip Scanner 3000 and gene expression intensities were calculated using the Affymetrix GeneChip Command Console software (AGCC). Affymetrix .CEL files were normalized using the RMA (Robust Multi-Array Analysis) algorithm within Partek Genomics Suite software, version 6.5 (Partek Inc., St. Charles, Mo.). Analysis of variance (ANOVA) and linear contrasts were used to identify differentially expressed genes using a larger set of samples including additional controls. Lists of genes differentially expressed between Npc1^{-/-} and control mice were generated at each time point, using a combination of thresholds for both uncorrected P-value and fold-change (P-value ≦0.05, and fold-change ≦−1.3 or ≧1.3). This gene selection method combining P-value and fold-change cutoff, was previously demonstrated to result in higher concordance degree of differentially expressed genes between different platforms, when compared with genes selected only on P-value ranking (Guo et al., Nat. Biotechnol. 24:1162-9 (2006); Shi et al., BMC Bioinformatics 9(Suppl 9): S10 (2008); and Shi et al., Nat. Biotechnol. 24:1151-61 (2006)). Pathway and GO-enrichment analysis was carried out using Partek and MetaCore software (GeneGo Inc., St. Joseph, Mich.).

Comparison with Other Microarray Studies

Three other microarray studies using either cerebella from the murine mouse model or human fibroblasts have been published (Reddy et al., PLoS One 1:e19 (2006); Liao et al., Brain Res. 1325:128-40 (2010); and De Windt et al., DNA Cell Biol. 26:665-71 (2007)). Gene symbols in the two human gene lists were converted to their murine orthologs using the Ensembl Biomart tool (http://www.ensembl.org/biomart/martview). Gene symbol lists were then compared in Excel to identify common genes with altered expression.

### Real-Time PCR

Total RNA (10 μg), from the same set of livers used for the microarray analysis, was reverse-transcribed into cDNA using a High-Capacity cDNA archive kit according to the manufacturer's instructions (Applied Biosystems, Carlsbad, Calif.). The following Taqman assays were used to assess the expression level of a few genes with an altered expression in the microarray data (Applied Biosystems, Carlsbad, Calif.): Npc1 (Mm00435283), Abcg1 (Mm01348250), Sqle (Mm00436772), Idi1 (Mm00836417), Cyp51 (Mm00490968), Lpl (Mm00434770), Hexa (Mm00599877), Mmp12 (Mm00500554), Hhip (Mm00469580), Rragd (Mm00546741), Gpnmb (Mm00504347), Itgax (Mm00498698), Itgb2 (Mm00434513), Ctss (Mm01255859), Cyba (Mm00514478), Cybb (Mm01287743), Lyz2 (Mm01612741), and Syngr1 (Mm00447433). Gapdh was used as reference (Taqman Rodent GAPDH control reagents; Applied Biosystems, Carlsbad, Calif.). All the different gene assays were first validated using serial dilutions of a control cDNA to check their efficiency rates in qPCR compared to Gapdh.

Quantitative real-time PCR was performed in 384-well plates with an Applied Biosystems 7900 real-time PCR system (Applied Biosystems, Carlsbad, Calif.). Each sample was analyzed in triplicate, using 50 ng of total cDNA for each reaction. The relative quantification of gene expression was performed with the comparative cycle number measured with the threshold method (C_{T}) (Livak and Schmittgen, Methods 25:402-8 (2001)), using the 1-week-old control samples as reference for quantification, and was plotted with mean and standard error of the mean (SEM) for each age- and genotype-group. An ANOVA with a Games-Howell correction was performed to assess the significance of the difference of means between control and mutant samples at each age.

### Patients

The study was approved by the Eunice Kennedy Shriver National Institute of Child Health and Human Development Institutional Review Board. Informed consent and, when appropriate, assent were obtained. Serum samples were obtained from 16 male and 14 female NPC1 patients participating in a Natural History trial (06-CH-0186) and 16 control individuals of similar age and gender distribution. Mean age of patients and controls were 7.9±5.8 years old and 9.1±4.2 years old respectively (P-value=0.3720). Phenotypic severity was determined using the severity scale developed by Yanjanin et al., Am. J. Med. Genet. B. Neuropsychiatr. Genet. 153B:132-40 (2010), and ranged from 0 to 40. The four-month-old female with a severity score of 0 has an unusual, severe liver presentation, with no neurological signs, and was therefore excluded from the statistical analyses. Eighteen (60%) of the patients were being treated off-label with miglustat, an inhibitor of glycosphingolipid synthesis. Serum samples from patients affected with Gaucher Disease, Infantile Neuronal Ceroid Lipofuscinosis, and both GM1 and GM2 gangliosidosis were obtained from other ongoing NIH trials.

### Enzyme-Linked Immunosorbant Assays

Serum levels of cathepsin D (CTSD; EMD Millipore, Billerica, Mass.), and galectin-3 (LGALS3; R&D systems, Minneapolis, Minn.) were measured in triplicate by ELISA, following manufacturers' instructions. Standards were prepared following manufacturers' instructions. A standard curve was generated by linear regression and polynomial regression for LGALS3 and CTSD, respectively. For LGALS3, a 1:3 dilution was performed for both control and patient serum with the calibrator diluent provided by the manufacturer. For CTSD, serum was not diluted for controls but diluted 1:2 for patients with the sample diluent reagent provided by the manufacturer. Serum from some NPC1 patients with high CTSD levels (>50 ng/mL) had to be diluted up to 10-fold in order to be within the linear range of the assay.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### INCORPORATION BY REFERENCE

All patents, publications, and CAS numbers mentioned in this specification are herein incorporated by reference to the same extent as if each independent patent and publication was specifically and individually indicated to be incorporated by reference.

1. A method for identifying a subject as having Niemann-Pick disease, type C (NPC), the method comprising
(a) detecting the level of a biomarker selected from the group comprising i) galectin-3 (LGALS3); ii) cathepsin D (CTSD); iii) LGALS3 and CTSD; and iv) LGALS3 and/or CTSD in combination with at least one additional NPC associated biomarker in a sample obtained from the subject; and
(b) comparing the level of the biomarker to a reference.

2. The method of claim 1, wherein the subject is identified as having NPC when the level of the biomarker is increased relative to the reference.

3. A method for identifying Niemann-Pick disease, type C (NPC) in a subject, the method comprising
(a) detecting the level of a biomarker selected from the group comprising i) galectin-3 (LGALS3); ii) cathepsin D (CTSD); iii) LGALS3 and CTSD; and iv) LGALS3 and/or CTSD in combination with at least one additional NPC associated biomarker in a sample obtained from the subject; and
(b) comparing the level of the biomarker to a reference.

4. The method of claim 3, wherein NPC is identified in the subject when the level of the biomarker is increased relative to the reference.

5. A method for characterizing the stage of neurological disease in a subject, the method comprising
(a) detecting the level of a biomarker selected from the group comprising i) galectin-3 (LGALS3); ii) cathepsin D (CTSD); iii) LGALS3 and CTSD; and iv) LGALS3 and/or CTSD in combination with at least one additional NPC associated biomarker in a sample obtained from the subject; and
(b) comparing the level of the biomarker to a reference.

6. The method of claim 5, wherein an increase in the level of the biomarker relative to the reference identifies the subject as having a later stage of neurological disease.

7. The method of claim 6, wherein the subject has Niemann-Pick disease, type C (NPC).

8. The method of claim 1, wherein the one additional NPC associated biomarker is selected from the group consisting essentially of a NPC associated protein, NPC associated lipid, and NPC associated oxysterol.

9. The method of claim 8, wherein the NPC associated protein is calbindin D, fatty acid binding protein 3, or fatty acid binding protein 7.

10. The method of claim 8, wherein the NPC associated oxysterol is 7-ketocholesterol or 3β,5α,6β-cholestane-triol.

11. The method of claim 1, wherein the biomarker is LGALS3.

12. The method of claim 1, wherein the biomarker is CTSD.

13. The method of claim 1, wherein the biomarker is LGALS3 and CTSD.

14. The method of claim 11, wherein the biomarker further comprises calbindin D, fatty acid binding protein 3, fatty acid binding protein 7,7-ketocholesterol, or 3β,5α,6β-cholestane-triol.

15-16. (canceled)

17. A method for identifying a subject as having Niemann-Pick disease, type C (NPC), the method comprising detecting the level of a biomarker selected from the group comprising galectin-3 (LGALS3) and cathepsin D (CTSD) in a sample obtained from the subject, or
A method for identifying Niemann-Pick disease, type C (NPC) in a subject, the method comprising detecting the level of a biomarker selected from the group comprising galectin-3 (LGALS3) and cathepsin D (CTSD) in a sample obtained from the subject.

18. (canceled)

19. The method of claim 17, wherein the subject is identified as having NPC when the level of the LGALS3 biomarker is at least about 10 ng/mL.

20-21. (canceled)

22. A method for monitoring Niemann-Pick disease, type C (NPC) therapy in a subject, the method comprising
(a) detecting the level of a biomarker selected from the group comprising i) galectin-3 (LGALS3); ii) cathepsin D (CTSD); iii) LGALS3 and CTSD; and iv) LGALS3 and/or CTSD in combination with at least one additional NPC associated biomarker in a sample obtained from the subject; and
(b) comparing the level of the biomarker to a reference.

23-25. (canceled)

26. A method for detecting an agent's therapeutic efficacy in a subject having Niemann-Pick disease, type C (NPC), the method comprising
(a) detecting the level of a biomarker selected from the group comprising i) galectin-3 (LGALS3); ii) cathepsin D (CTSD); iii) LGALS3 and CTSD; and iv) LGALS3 and/or CTSD in combination with at least one additional NPC associated biomarker in a sample obtained from the subject; and
(b) comparing the level of the biomarker to a reference.

27-32. (canceled)

33. The method of claim 1, wherein the subject is human.

34. The method of claim 1, wherein the sample is a biological fluid selected from the group consisting of blood, blood serum, plasma, cerebrospinal fluid, saliva, and urine.

35. (canceled)

36. (canceled)

37. A kit for aiding the diagnosis of Niemann-Pick disease, type C (NPC), the kit comprising at least one reagent capable of detecting or capturing galectin-3 (LGALS3) and/or cathepsin D (CTSD), or
A kit for aiding the diagnosis of Niemann-Pick disease, type C (NPC), the kit comprising an adsorbent that retains (LGALS3) and/or cathepsin D (CTSD).

38. The kit of claim 37, wherein the reagent is an antibody that specifically binds to LGALS3 and/or CTSD.

38-48. (canceled)
